(19) United States
(12) Patent Application Publication
(10) Pub. No.: US 2026/0317288 A1
(43) Pub. Date: Oct. 15, 2026

(54) ONE-STEP SYNTHESIS OF BETA-ALKYLIDENE-GAMMA-LACTONES

(71) Applicant: THE SCRIPPS RESEARCH INSTITUTE, La Jolla, CA (US)

(72) Inventors: Jin-Quan YU, San Diego, CA (US); Tao SHENG, San Diego, CA (US)

(21) Appl. No.: 18/881,338

(22) PCT Filed: Jul. 5, 2023
(86) PCT No.: PCT/US23/69645

§ 371 Date: Jan. 6, 2025

Related U.S. Application Data
(60) Provisional application No. 63/358,381, filed on Jul. 5, 2022.

(51) Int. Cl. C07D 307/88; C07C 67/30; C07D 307/58; C07D 307/93

(52) U.S. Cl. CPC C07D 307/88; C07C 67/30; C07D 307/58; C07D 307/93

(57) ABSTRACT

This application discloses novel tandem dehydrogenation-olefination-lactonization reactions for the preparation of β-alkylidene-γ-lactones using bidenate oxime ether-pyridone and morpholine-pyridone ligands for β,γ-dehydrogenation and subsequent vinyl C—H olefination of fee carboxylic acids, providing a unique method for the structural diversification of aliphatic acids containing α-quaternary centers through the sequential functionalization of two β-C—H bonds and one γ-C—H bond.

### CROSS-REFERENCE TO RELATED APPLICATION

This application claims the benefit of U.S. Provisional Application No. 63/358,351, filed on Jul. 5, 2022, which is incorporated herein by reference in its entirety.

### GOVERNMENT SUPPORT

This invention was made with government support under GM084019 awarded by the National Institutes of Health. The government has certain rights in the invention.

### FIELD OF THE INVENTION

This application discloses novel tandem dehydrogenation-olefination-lactonization reactions for the preparation of β-alkylidene-γ-lactones using bidentate oxime ether-pyridone and morpholine-pyridone ligands for β,γ-dehydrogenation and subsequent vinyl C—H olefination of free carboxylic acids, providing a unique method for the structural diversification of aliphatic acids containing α-quaternary centers through the sequential functionalization of two β-C—H bonds and one γ-C—H bond.

### BACKGROUND OF THE INVENTION

The desaturation of carbonyl compounds is an important transformation in organic chemistry, owing to the versatile reactivity of the double bond in downstream applications.¹ Despite significant advances in the desaturation of ketone and aldehyde substrates, aliphatic carboxylic acids, which are ubiquitous and highly versatile motifs, are less amenable to dehydrogenation reactions. In 2017, an elegant method for the α,β-desaturation of free carboxylic acids via zinc enediolates, using an allyl-Pd catalyst and allyl acetate as the stoichiometric oxidant was reported.² More recently developed were a pair of Pd^{II}-catalyzed α,β-dehydrogenation reactions of free carboxylic acids through β-methylene C—H activation, delivering α,β-unsaturated carboxylic acids or γ-alkylidene butenolides (Scheme 1A).³ The success of this protocol hinged on the use of bidentate pyridine-pyridone ligands with different bite angles to overcome product inhibition: (1) the five-membered chelating ligand L6 was designed to preferentially activate C(sp³)-H bonds over the vinyl C—H bonds of α,β-unsaturated carboxylic acid intermediates; (2) the six-membered chelating ligand L5 promoted a tandem vinyl C—H activation and coupling with alkynyl bromide. This β-C—H activation-enabled α,β-dehydrogenation process prompted us to investigate whether this strategy could be exploited to promote β,γ-dehydrogenation in the absence of α-C—H bonds, thereby achieving the functionalization of remote γ-methylene C—H bonds, a feat unachievable using enolate chemistry. An early study from our group in 2008 demonstrated that, in the presence of oxazoline directing groups (DGs) and stoichiometric quantities of Pd, a complex exhibiting β,γ-dehydrogenation could be isolated in 63% yield (Scheme 1B).⁴ Although a catalytic protocol was developed, it suffered from limited scope (a single cyclopentanecarboxylic acid example), low efficiency, and required the use of exogenous oxazoline DGs (Scheme 1B). Therefore, the regioselective β,γ-dehydrogenation of native substrates such as carboxylic acids via C—H activation remains a significant challenge.⁵

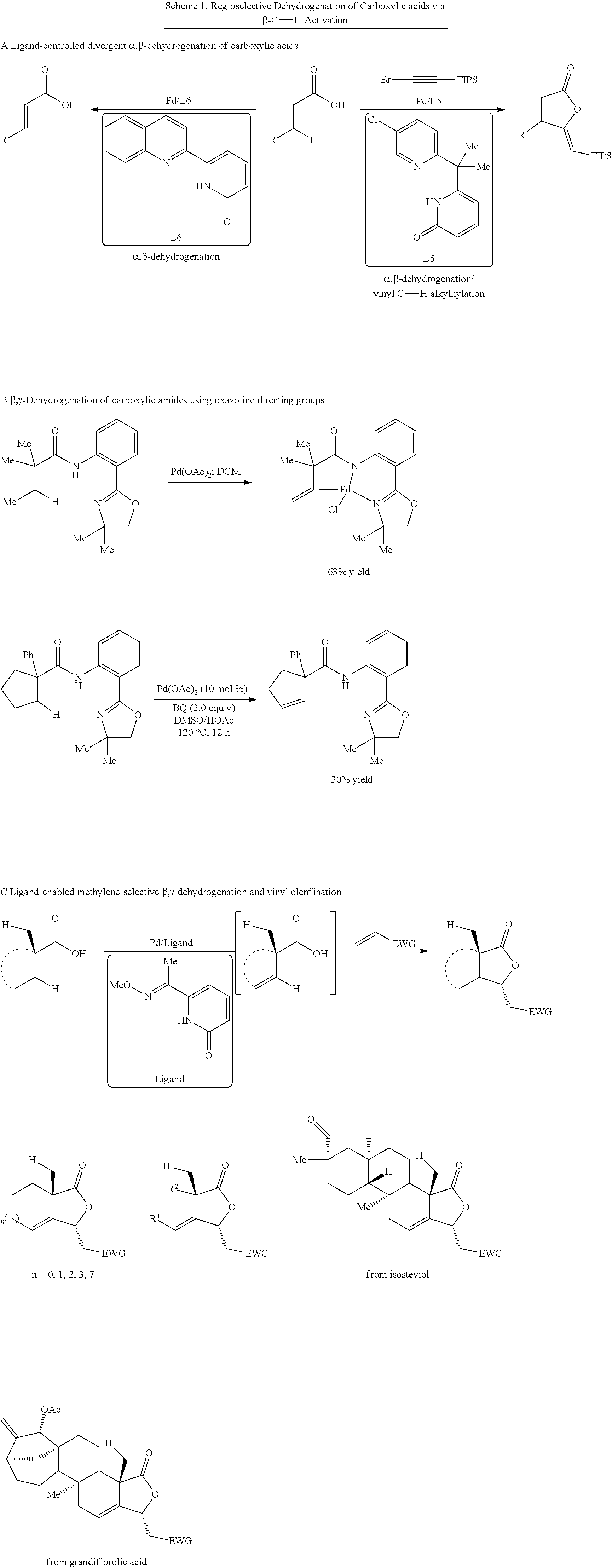

Achieving regioselective β,γ-dehydrogenation necessitates the preferential activation of methylene C—H bonds, despite the possible presence of more accessible α-methyl groups.⁶ Recent efforts yielded an efficient synthesis of benzocyclobutenes through the methylene-selective C—H arylation of ketones.⁷ Computational studies showed that this selectivity for methylene C—H activation was driven by ligand substrate interactions and the geometry of the Pd during the competing oxidative addition events. Despite the elegance of this intramolecular C—H arylation approach, designing ligands to obtain selectivity for methylene C—H bonds over methyl C—H bonds remains a formidable challenge. Thus, there remains a need in the field for more widely applicable and efficient methods of regioselective β,γ-dehydrogenation.

Ligand-enabled Pd-catalyzed regioselective α,β-dehydrogenation of carbonyl compounds via β-methylene C—H activation has recently emerged as a promising transformation. Herein disclosed are methods of tandem Pd-catalyzed β,γ-dehydrogenation and vinyl C—H olefination of free carboxylic acids for the synthesis of a wide range of β-alkylidene-γ-lactones (Scheme 1C).

### SUMMARY OF THE INVENTION

Herein, disclosed is the realization of β,γ-dehydrogenation and subsequent vinyl C—H olefination reactions of free carboxylic acids, thus providing a unique method for the structural diversification of aliphatic acids containing α-quaternary centers through sequential functionalizations of two β-C—H bonds and one γ-C—H bond. This tandem dehydrogenation-olefination-lactonization reaction offers a one-step preparation of β-alkylidene-γ-lactones, which are often difficult to prepare through conventional methods, from inexpensive and abundant free aliphatic acids. A variety of free aliphatic acids, such as isosteviol and grandiflorolic acid natural products, and olefins are compatible with the reported protocol. The newly-designed bidentate oxime ether-pyridone and morpholine-pyridone ligands are crucial for this tandem reaction to proceed. Notably, these ligands also enable preferential methylene C—H activation over the previously reported, competing process of methyl C—H bond olefination.

The application provides a method of forming a β-alkylidene-γ-lactone, comprising treating an α-substituted carboxylic acid with a vinyl reagent containing an electron withdrawing group (EWG) in the presence of a Pd source/Ligand (L) catalyst.\

The application provides the above method, wherein the method of forming a β-alkylidene-γ-lactone occurs according to the following reaction scheme:

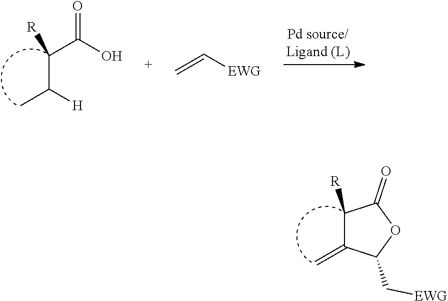

wherein

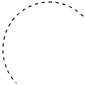

represents a substituted or unsubstituted C₅-C₁₂ ring system;
R is optionally substituted —(C₁-C₆)alkyl, aryl, —(C₁-C₆)haloalkyl, —O(C₁-C₆)alkyl, cyclocalkyl, —(C₁-C₆)alkylcycloalkyl, or —(C₁-C₆)alkylaryl;
EWG is —CO₂Bn, —CN, —CO₂R′, —C(═O)R′, —SO₂R′, or —P(O)(OR′)₂; and
   each R′ is independently optionally substituted —(C₁-C₁₂)alkyl, —O(C₁-C₁₂)alkyl,
   cycloalkyl, heterocycloalkyl, —N((C₁-C₆)alkyl)₂, or Ph.

The application provides the above method, wherein the method of forming a β-alkylidene-γ-lactone occurs according to the following reaction scheme:

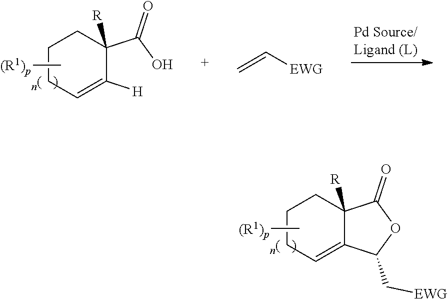

wherein:
R is optionally substituted —(C₁-C₆)alkyl, aryl, —(C₁-C₆) haloalkyl, —O(C₁-C₆)alkyl, cyclocalkyl, —(C₁-C₆)alkylcycloalkyl, or —(C₁-C₆)alkylaryl;
EWG is —CO₂Bn, —CN, —CO₂R′, —C(═O)R′, —SO₂R′, or —P(O)(OR′)₂;
   each R′ is independently optionally substituted —(C₁-C₁₂)alkyl, —O(C₁-C₁₂)alkyl,
   cycloalkyl, heterocycloalkyl, —N((C₁-C₆)alkyl)₂, or Ph;
each R¹ is independently optionally substituted —(C₁-C₆)alkyl or —O(C₁-C₆)alkyl;
n is 0-7; and
p is 0-4.

The application provides the above methods, wherein the Ligand (L) is selected from:

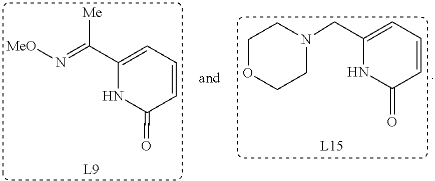 [SMILES: CO/N=C(\C)c1cccc(=O)[nH]1 ; O=c1cccc(CN2CCOCC2)[nH]1]

The application provides the above methods, wherein the method of forming a β-alkylidene-γ-lactone occurs according to the following reaction scheme:

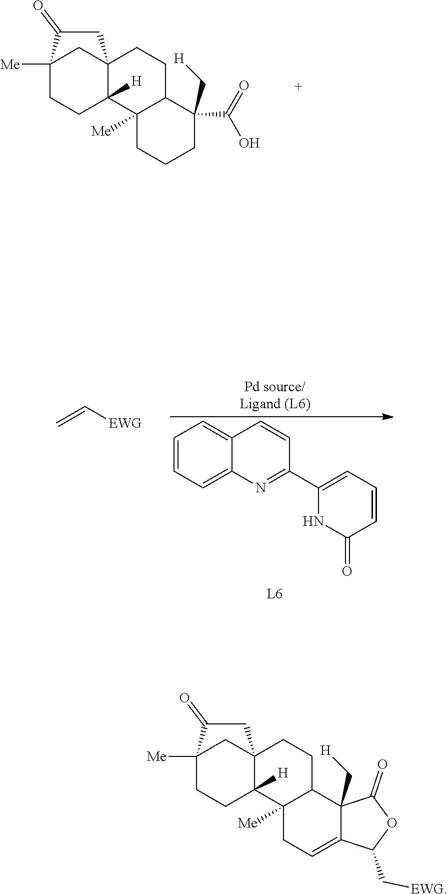

The application provides the above methods, wherein the method of forming a β-alkylidene-γ-lactone occurs according to the following reaction scheme:

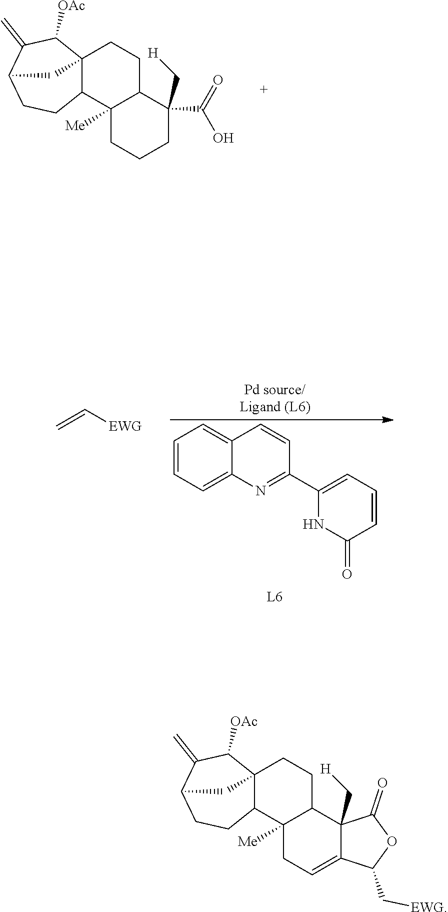

The application provides the above methods, wherein the method of forming a β-alkylidene-γ-lactone occurs according to the following reaction:

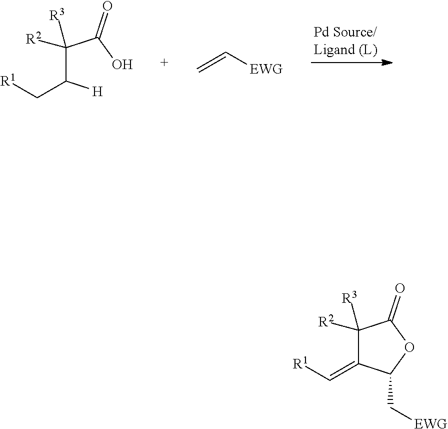

wherein:
R¹ is H, —(C₁-C₆)alkyl, or —(C₁-C₆)alkyl O(C₁-C₆)alkyl;
R² and R³ are independently —(C₁-C₆)alkyl, —(C₁-C₆)alkyl Ph, —(C₁-C₆)alkylcycloalkyl, (C₁-C₆)alkyl O(C₁-C₆)alkyl, or halo(C₁-C₆)alkyl;
EWG is —CO₂Bn, —CN, —CO₂R′, —C(═O)R′, —SO₂R′, or —P(O)(OR′)₂; and
   each R′ is independently optionally substituted —(C₁-C₁₂)alkyl, —O(C₁-C₁₂)alkyl,
   cycloalkyl, heterocycloalkyl, —N((C₁-C₆)alkyl)₂, or Ph.

### DETAILED DESCRIPTION OF THE INVENTION

Herein, reported is a tandem Pd-catalyzed β,γ-dehydrogenation and vinyl C—H olefination of free carboxylic acids for the synthesis of a wide range of β-alkylidene-γ-lactones (Scheme 1C). The key to success was the design of bidentate oxime ether-pyridone and morpholine-pyridone ligands to achieve double functionalization through sequential methylene and vinyl C—H activation. This protocol has several key advantages over other C—H functionalization reactions: (1) the use of native carboxylic acids as substrates without the installation of exogenous directing groups, (2) exclusive β-methylene selectivity in the presence of primary β-C—H bonds, (3) the functionalization of γ-methylene C—H bonds, and (4) compatibility with a broad range of carboxylic acids such as isosteviol and grandiflorolic acid natural products.

An early study from our group disclosed a single example of β,γ-dehydrogenation and subsequent vinyl C—H olefination of 1-propylcyclohexane-1-carboxylic acid in low yield (41%) using five-membered chelating pyridine-pyridone ligand L6 (Scheme 2).³ Despite extensive efforts to improve this reaction, yields remained low and other cyclic carboxylic acids (e.g. cyclopentane, cycloheptane, and cyclooctane) and acyclic substrates were unreactive (Scheme 2). We decided to further explore this curious reactivity using the commercially available substrate 1-methyl-1-cyclohexanecarboxylic acid 1a, since such α-methylated carboxylic acids are widespread among natural products such as terpenes. Under the reported conditions using L6, we were able to observe a 22% ¹H NMR (nuclear magnetic resonance) yield for the product 3a resulting from the desired tandem β,γ-dehydrogenation, vinyl C—H olefination, and lactonization sequence. Extensive screening of reaction conditions revealed that the yield could be improved to 66% through two crucial modifications: a 10:1 HFIP:MeCN solvent mixture and use of KF as a base (See Table S3 and Table S4 in the EXAMPLES).

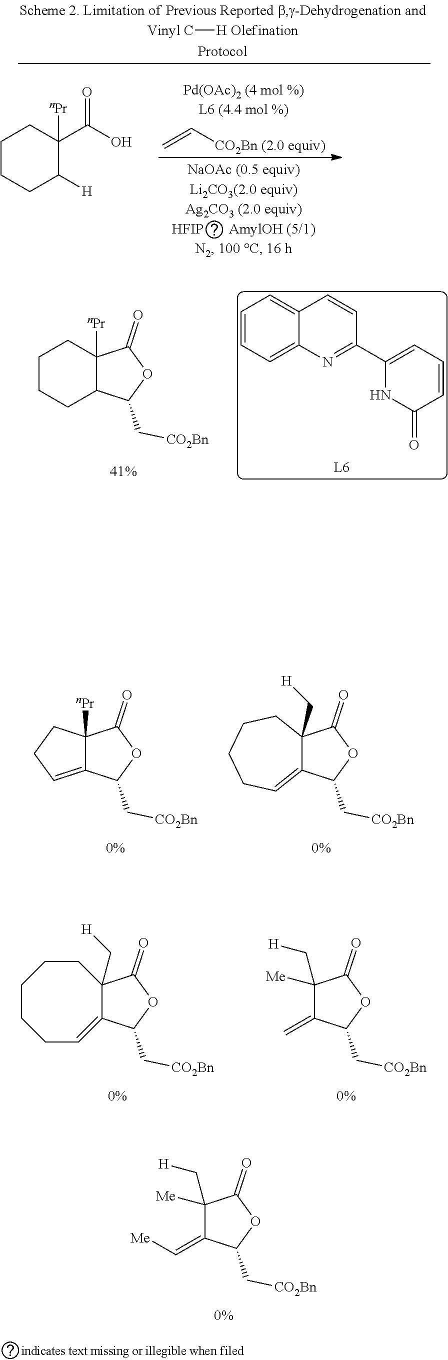

Next searched for were ligands that could further improve the reactivity of the catalyst (Table 1).^{8,9} A variety of different ligand classes previously developed to enable the C—H functionalization of free carboxylic acids, such as pyridine L1,^{9a} 2-pyridone L2,9% and mono-N-protected amino acid (MPAA) ligand L3,^{8b} promoted primary C—H activation to deliver the undesired β-olefination product 3a′ in 10-34% yields. The bidentate thioether ligand L4, developed for the β-olefination of free aliphatic acids, afforded a mixture of methylene and methyl C—H activation products in 10% and 24% yields, respectively.^{5f} Modifications to the bite angle and electronic and steric properties of ligand L6 failed to identify a more potent ligand (e.g. 20% yield of 3a with L5, 60% with L7, and 66% with L6). At this point, previous computational results, which had revealed that the 2-pyridone moiety was likely serving as an internal base to promote C—H bond cleavage, in a similar manner to an NHAc group were considered.^{3,10} Bearing this in mind, it was concluded that five-membered chelate analogs of L6, which kept the pyridone intact but replaced the pyridine moiety with other σ-donors, merited further investigation. First prepared was a range of oxime ether-pyridone ligands (L8-L11), inspired by the well-documented directing group ability of oxime-ethers.¹¹ Among these, a significant improvement in reactivity using methyl ketone oxime ether ligand L9 with 84% ¹H NMR yield (80% isolated yield) of 3a and moderate diastereoselectivity (4/1) was obtained. Introducing sterically bulky groups was detrimental to the reactivity of this new catalyst (e.g. 6% with L10 and 0% with L11). Because of the more diffuse lone pair of the sp³ nitrogen relative to the sp² nitrogen of pyridine, we also designed and tested a series of tertiary amine-pyridone ligands (L12-L15), with morpholine-pyridone ligand L15 affording a comparably high yield (78%).^{5e} Control experiments showed that only the undesired β-C—H olefination product 3a′ (deriving from methyl C—H activation) was obtained in the absence of these ligands, indicating the importance of the bidentate oxime ether-pyridone L9 and morpholine-pyridone L15 ligands for the observed reactivity and exclusive methylene selectivity.

TABLE 1
Investigation of Ligands for the B [symbol] γ-Dehydrogenation and Vinyl C—H Olefination of Free
Carboxylic Acids^{a,b}

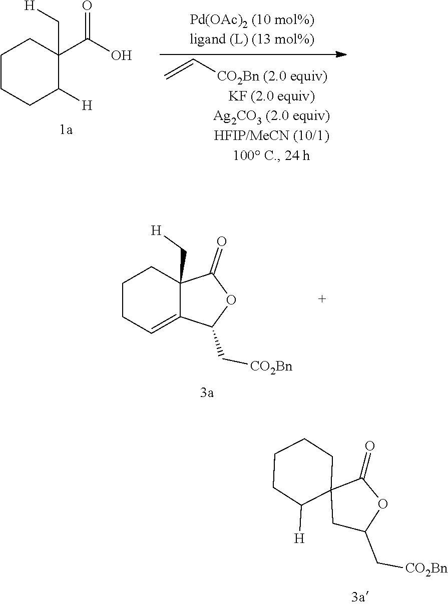

| | |
|---|---|
| w/o ligand(L) 3a/3a′ 0%/12% | 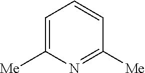 |
| | L1, 0%/10% |
| 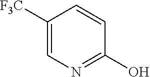 [SMILES: Oc1ccc(C(F)(F)F)cn1] L2, 0%/26% | 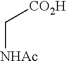 L3, 0%/34% |
| 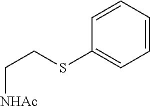 [SMILES: CC(=O)NCCSc1ccccc1] L4, 10%/24% | 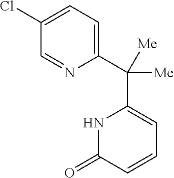 [SMILES: CC(C)(c1ccc(Cl)cn1)c1cccc(=O)[nH]1] |
| | L5, 20%/0% |
| 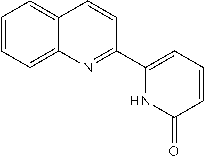 [SMILES: O=c1cccc(-c2ccc3ccccc3n2)[nH]1] | 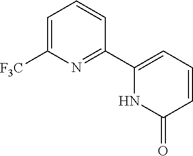 [SMILES: O=c1cccc(-c2cccc(C(F)(F)F)n2)[nH]1] |
| L6, 66%/0% | L7, 6 [symbol] %/0% |
| 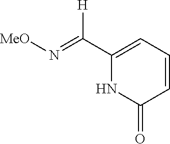 [SMILES: CO/N=C/c1cccc(=O)[nH]1] L8, 50%/10% | 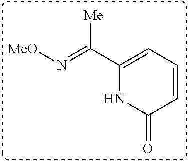 [SMILES: CO/N=C(\C)c1cccc(=O)[nH]1] L9, 84%/10% ( [symbol] ) |
| 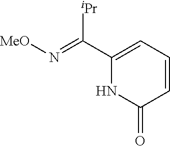 [SMILES: CO/N=C(/c1cccc(=O)[nH]1)C(C)C] L10, 6%/ [symbol] % | 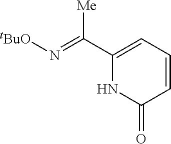 [SMILES: C/C(=N\OC(C)(C)C)c1cccc(=O)[nH]1] L11, 0%/0% |
| 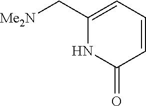 [SMILES: CN(C)Cc1cccc(=O)[nH]1] | 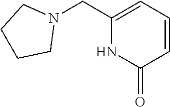 [SMILES: O=c1cccc(CN2CCCC2)[nH]1] |
| L12, 22%/0% | L13, 28%/0% |
| 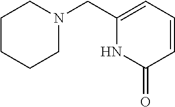 [SMILES: O=c1cccc(CN2CCCCC2)[nH]1] L14, 42%/0% | 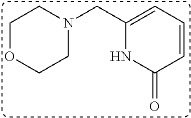 [SMILES: O=c1cccc(CN2CCOCC2)[nH]1] L15, 76%/0% |

^{a}Conditions: 1a (0.1 mmol), benzyl acrylate 2a (0.2 mmol), Pd(OAc)₂ (10 mol %), ligand (L)
(13 mol %), KF (2.0 equiv), Ag₂CO₃ (2.0 equiv), HFIP/MeCN (1.0 mL/0.1 mL), 100 °C, 24 h.
^{b}The yields of 3/3a′ were determined by ¹H NMR analysis of the crude product using CH₂Br₂ as
the internal standard.
The dr values of 3a were determined by ¹H NMR analysis of the crude product (~4/1).
^{c}Isolated yield of 3a. [symbol]
indicates data missing or illegible when filed

With the optimal ligand and reaction conditions in hand, next evaluated was the substrate scope of the tandem β,γ-C—H dehydrogenation-vinyl olefination reaction (Table 2). A wide range of free cyclic aliphatic acids including six-(1a-s), seven-(1t-x), eight-(1y), and twelve-membered (1z) rings was compatible, affording the fused γ-lactone products (3a-3z) in moderate to good yields (45-82%) with good diastereoselectivities (up to >20/1), albeit cyclopentane substrate 1aa was low yielding (3aa, 20%). The observed selective functionalization of methylene C—H bonds in the presence of primary (3a, 3l, 3m, 3r-t, 3y, and 3z) or aryl (3f-i) C—H bonds is unique compared to the vast majority of C—H activation reactions. A variety of functionalities such as chloro (3j), trifluoromethyl (3k), methoxy (3i, 3q, and 3x), ketone (3r), and allylic acetate (3s) were all well-tolerated, with the chloro moiety (3j) serving as a useful synthetic handle for subsequent derivatization. In addition to the carboxylic acids containing an α-quaternary center, cyclohexanecarboxylic acids bearing substitution at the 3 or 4 position (11 and 1m-q) consistently provided the corresponding products (31-q) in good yields (60-70%). The utility of this protocol has been further demonstrated by the late-stage functionalization of complex natural products such as isosteviol 1r and grandiflorolic acid 1s,¹² thus providing densely functionalized γ-lactones with potential biological activity in synthetically useful yields (58% and 52%, respectively). The reaction can be reliably scaled up to a 1.0 mmol scale, delivering 3c in 76% yield.

Acyclic carboxylic acids containing less reactive methylene C—H bonds were compatible with the optimal catalyst, delivering the corresponding γ-lactones (5a-1) in moderate to good yields (34-74%) with exclusive methylene selectivity (Table 3). Our previous study reported that aliphatic acids bearing an α-gem-dimethyl group (4a-c) or a single methyl group (4d) preferentially reacted at the methyl C—H bonds using bidentate thioether ligand L4.^{5f} The newly developed ligand scaffolds reversed this inherent methyl selectivity to methylene, highlighting the importance of ligand design for chemoselective (i.e. methylene vs. methyl) C(sp³)-H functionalization. The facile construction of β-methylene-γ-butyrolactones (5a and 5d-1) is extremely valuable, due to their widespread presence among many furanoid terpenes, and the corresponding dearth of existing methods for the synthesis of this scaffold.¹³ A wide range of functional groups, such as methoxy (5c and 5j), cyclohexane (5i), fluoro (5k), and chloro (51), was tolerated. This protocol can be conducted on a 1.0 mmol scale to afford 5a in 56% yield.

TABLE 2
Cyclic Carboxylic Acid Scope for the B,γ-Dehydrogenation and Vinyl C—H Olefination of
Free Carboxylic Acid^{a,b}

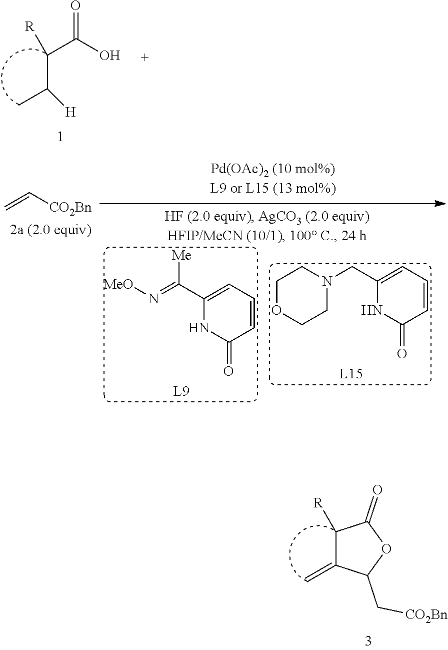

| | |
|---|---|
| 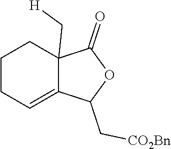 [SMILES: CC12CCCC=C1C(CC(=O)OCc1ccccc1)OC2=O] | 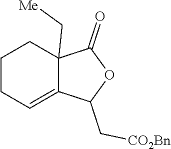 [SMILES: CCC12CCCC=C1C(CC(=O)OCc1ccccc1)OC2=O] |
| 3a, 80%, dr = 4/1 | 3b, 78%, dr = 9/1 |
| 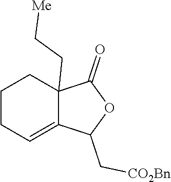 [SMILES: CCCC12CCCC=C1C(CC(=O)OCc1ccccc1)OC2=O] | 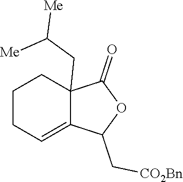 [SMILES: CC(C)CC12CCCC=C1C(CC(=O)OCc1ccccc1)OC2=O] |
| 3c, 80% [symbol] (76%), dr = 10/1 | 3d, 66%, dr = 8/1 |
| 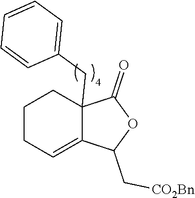 | 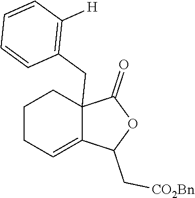 [SMILES: O=C(CC1OC(=O)C2(Cc3ccccc3)CCCC=C12)OCc1ccccc1] |
| 32e, 72%^{d}, dr = 6/1 | 3f, 75% [symbol] , dr = 5/1 |
| 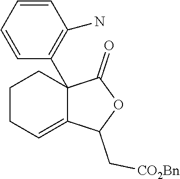 [SMILES: Nc1ccccc1C12CCCC=C1C(CC(=O)OCc1ccccc1)OC2=O] 3g, 62%, dr = 7/1 | 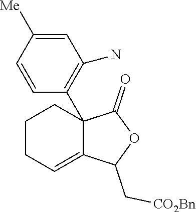 [SMILES: Cc1ccc(C23CCCC=C2C(CC(=O)OCc2ccccc2)OC3=O)c(N)c1] |
| | 3h, 56%^{d}, dr = 6/1 |
| 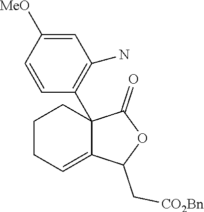 [SMILES: COc1ccc(C23CCCC=C2C(CC(=O)OCc2ccccc2)OC3=O)c(N)c1] | 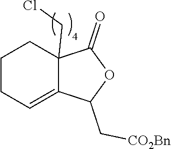 3j, 70%, dr = 6/1 |
| 3i, 42% [symbol] , dr = 7/1 | |
| 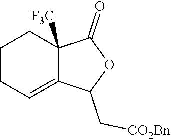 [SMILES: O=C(CC1OC(=O)[C@@]2(C(F)(F)F)CCCC=C12)OCc1ccccc1] 3k, 40%^{d}, dr = 10/1 | 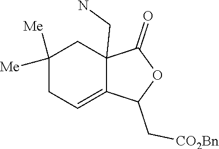 [SMILES: CC1(C)CC=C2C(CC(=O)OCc3ccccc3)OC(=O)C2(CN)C1] 3l, 66%, dr = 6/1 |
| 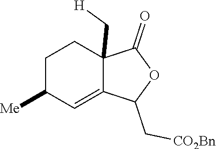 [SMILES: C[C@@H]1C=C2C(CC(=O)OCc3ccccc3)OC(=O)[C@@]2(C)CC1] 3m, 60% [symbol] , dr = 7/1 | 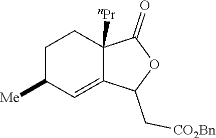 3n, 66%, dr = 8/1 |
| 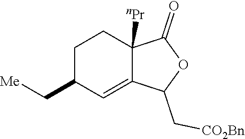 | 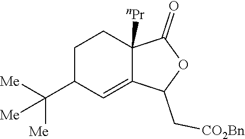 |
| 3o, 66%z,899 , dr = 11/1 | 3p, 70% [symbol] , dr = 10/1 |
| 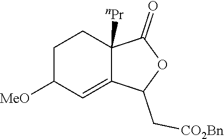 3q, 62%, dr = 11/1 | 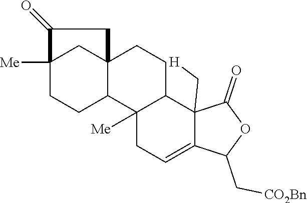 [SMILES: CC12C(=O)OC(CC(=O)OCc3ccccc3)C1=CCC1(C)C2CC[C@]23CC(=O)[C@](C)(CCC12)C3] |
| | from [symbol] |
| | 3r, [symbol] , dr = 13/1 |
| 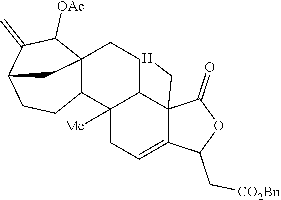 [SMILES: C=C1C(OC(C)=O)C23CCC4C5(C)C(=O)OC(CC(=O)OCc6ccccc6)C5=CCC4(C)C2CC[C@H]1C3] | 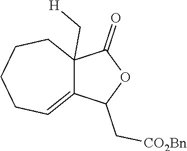 [SMILES: CC12CCCCC=C1C(CC(=O)OCc1ccccc1)OC2=O] 3t, [symbol] , dr = 2/1 |
| from [symbol] acid | |
| 3s, 52% [symbol] , dr = 10/1 | |
| 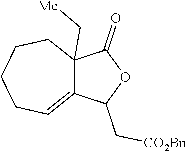 [SMILES: CCC12CCCCC=C1C(CC(=O)OCc1ccccc1)OC2=O] 3u, 62% [symbol] , dr = 7/1 | 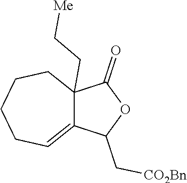 [SMILES: CCCC12CCCCC=C1C(CC(=O)OCc1ccccc1)OC2=O] |
| | 3v, 70%, dr = [symbol] /1 |
| 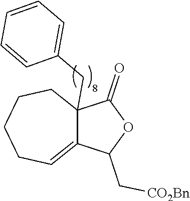 | 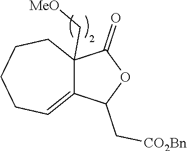 3x, 76%, dr = 2/1 |
| 3w, 60%, dr = 4/1 | |
| 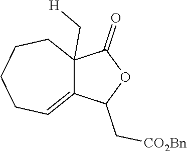 [SMILES: CC12CCCCC=C1C(CC(=O)OCc1ccccc1)OC2=O] | 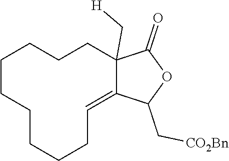 [SMILES: CC12CCCCCCCCC/C=C/1C(CC(=O)OCc1ccccc1)OC2=O] |
| 3y, 55%, dr = 1/1 | 3x, 45%, dr = 14/1 |
| 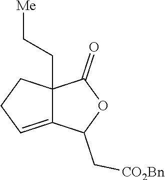 [SMILES: CCCC12CCC=C1C(CC(=O)OCc1ccccc1)OC2=O] | |
| 3aa, 20%^{e}, dr = 20/1 | |

^{a}Conditions: 1 (0.1 mmol), benzyl acrylate 2a (0.2 mmol), Pd(OAc)₂ (10 mol %), L9 (13 mol %), KF
(2.0 equiv), Ag₂CO₃ (2.0 equiv), HFIP/MeCN (1.0 mL/0.1 mL), 100 °C, 24 h.
^{b}Isolated yields.
The dr values were determined by ¹H NMR analysis of the crude product.
^{c}LiF (1.0 equiv) and HFIP/MeCN (1.0 mL/0.15 mL) were used.
^{d}L15 was used instead of L9.
^{e}L6 was used instead of L9.
^{f}The reaction was run on a 1.0 mmol scale. [symbol]
indicates data missing or illegible when filed

TABLE 3
Acyclic Carboxylic Acid Scope for the β [symbol] γ-Dehydrogenation and Vinyl C—H
Olefination of Free Carboxylic Acids^{a,b}

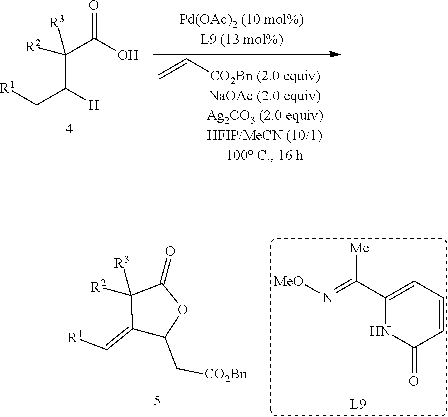

| | |
|---|---|
| 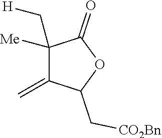 [SMILES: C=C1C(CC(=O)OCc2ccccc2)OC(=O)C1(C)C] | 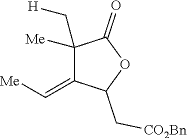 [SMILES: C/C=C1/C(CC(=O)OCc2ccccc2)OC(=O)C1(C)C] |
| 5a, 62% [symbol] ](56% [symbol] ) | 5b, 40%^{d} |
| 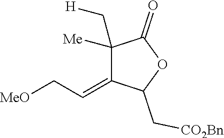 [SMILES: COC/C=C1/C(CC(=O)OCc2ccccc2)OC(=O)C1(C)C] | 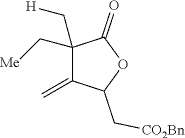 [SMILES: C=C1C(CC(=O)OCc2ccccc2)OC(=O)C1(C)CC] |
| 5c, 38% | 5d, 46%, dr = 1.5/1 |
| 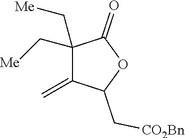 [SMILES: C=C1C(CC(=O)OCc2ccccc2)OC(=O)C1(CC)CC] | 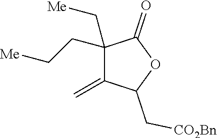 [SMILES: C=C1C(CC(=O)OCc2ccccc2)OC(=O)C1(CC)CCC] |
| 5e, 74% | 5f, 54% [symbol] , dr = 1.5/1 |
| 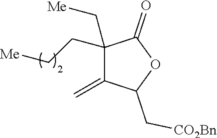 | 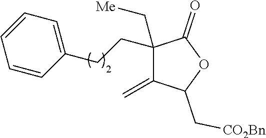 |
| 5g, 62%, dr = 2/1 | 5h, 42%, dr = 2.5/1 |
| 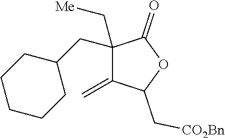 [SMILES: C=C1C(CC(=O)OCc2ccccc2)OC(=O)C1(CC)CC1CCCCC1] | 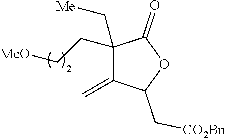 |
| 5i, 34%, dr = 1/1 | 5j, 56%, dr = 2/1 |
| 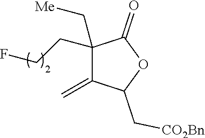 | 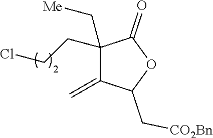 |
| 5k, 44%, dr = 2/1 | 5l, 42%, dr = 2/1 |

^{a}Conditions: 1 (0.1 mmol), benzyl acrylate 2a (0.2 mmol), Pd(OAc)2 (10 mol %), L9 (13 mol %),
NaOAc (2.0 equiv), Ag₂CO₃ (2.0 equiv), HFIP/MeCN (9.5 mL/0.05 mL), 100 °C, 16 h.
^{b}Isolated yields.
The dr values were determined by ¹H NMR analysis of the crude product.
^{c}L15 was used instead of L9.
^{d}Pd(OAc)₂ (15 mol %) and L9 (16 mol %) were used.
^{e}L6 was used instead of L9.
^{f}The reaction was run on a 1.0 mmol scale. [symbol]
indicates data missing or illegible when filed

Next, the scope of the olefin coupling partners was evaluated using 1a or 1c as the model acid substrate (Table 4). In addition to various acrylate derivatives (2a-h), a broad range of Michael acceptors, such as acrylonitrile (2i), acrylamide (2j and 2k), vinyl sulfone (21 and 2m), vinyl sulfonamide (2n), and vinyl phosphonate (20), were compatible with the optimal catalyst, providing the desired β-alkylidene-γ-lactone products (6a-o) in good yields (54-84%). Dimethyl fumarate 2h, a challenging coupling partner in C—H olefination reactions, was well-tolerated to deliver the corresponding product 6 h in 59% yield.

TABLE 4
Olefin Scope for the β [symbol] -Dehydrogenation and Vinyl C-H Olefination of Free Carboxylic
Acids^{a,b}

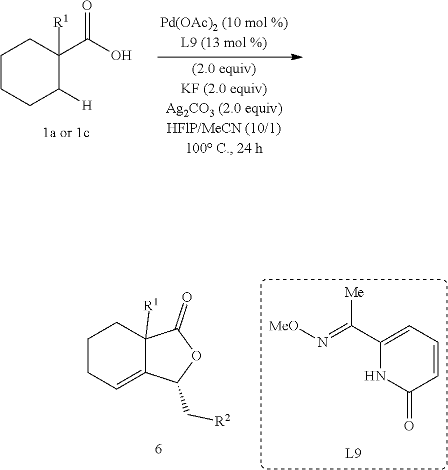

| | | | |
|---|---|---|---|
| 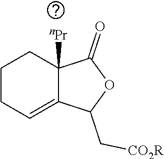 | 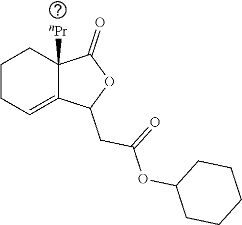 | 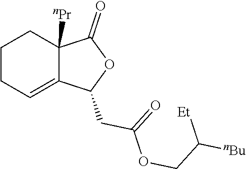 | 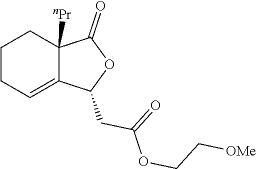 |
| 6a. R = Me [symbol] | 6d. 76% [symbol] | 6e. 78% [symbol] | 6f. 84% [symbol] |
| 6b. R = Et, 76% [symbol] | | | |
| 6c. R = [symbol] | | | |
| 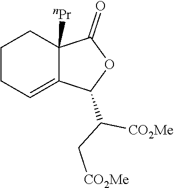 | 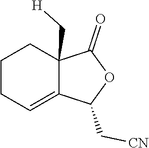 [SMILES: C[C@]12CCCC=C1[C@@H](CC#N)OC2=O] | 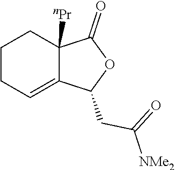 | 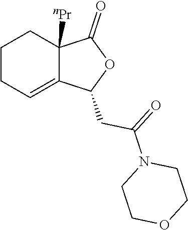 |
| 6h. 59% [symbol] | 6i, 60% [symbol] | 6j, 54% | 6k. 59% [symbol] |
| 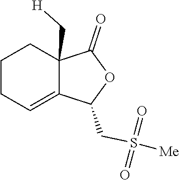 [SMILES: C[C@]12CCCC=C1[C@@H](CS(C)(=O)=O)OC2=O] | 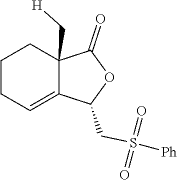 [SMILES: C[C@]12CCCC=C1[C@@H](CS(=O)(=O)c1ccccc1)OC2=O] | 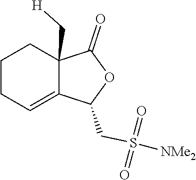 [SMILES: CN(C)S(=O)(=O)C[C@H]1OC(=O)[C@@]2(C)CCCC=C12] | 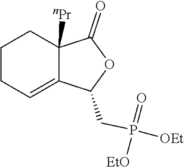 |
| 6l, 72% [symbol] | 6m, 61%, dr = [symbol] | 6n, [symbol] | 6o, 56% dr = 28/1 |

^{a}Conditions: 1a or 1c (0.1 mmol), olefin 2 (0.2 mmol), Pd(OAc)₂ (10 mol%), L9 (13 mol%), KF (2.0 equiv), Ag₂CO₃ (2.0 equiv), HFIP/MeCN (1.0 mL/0.1 mL), 100° C., 24 h.
^{b}Isolated yields. The dr values were determined by ¹H NMR analysis of the crude product.
^{c}LiF (1.0 equiv) and HFIP/MeCN (1.0 mL/0.15mL) were used. [symbol]
indicates data missing or illegible when filed

To highlight the synthetic applications of these methods, γ-lactone products 3c and 5a were then successfully transformed into several structurally distinct products (Scheme 3). Opening of γ-lactone 3c using NaOH through retro-Michael addition to unmask the olefin with subsequent benzyl protection afforded a synthetically useful diene product 7a in 83% yield. The [4+2] cycloaddition between diene 7a and o-silylaryl triflates provided tricyclic compound 7b in high yield (88%).¹⁴ Removal of the Bn protecting group in γ-lactone 5a in the presence of HCl gave the free carboxylic acid 7c in high yield (94%), with the terminal olefin remaining untouched. To investigate the potential biological activity of this β-methylene γ-lactone, an aniline containing an alkynyl group for protein labeling can be subsequently coupled with 7c to deliver 7d in 82% yield.

To investigate the role of the ligand in this cascade reaction, control experiments were conducted under either the standard or ligandless conditions (Scheme 4). First, under the standard conditions without an acrylate coupling partner, we observed the formation of β,γ-dehydrogenation product 3a″ in 18% yield; 3a″ was not formed under the ligandless conditions. These results highlighted the crucial role of the bidentate ligand for the dehydrogenation process and indicated that product inhibition likely contributes to the low yield of the reaction. Next, using dehydrogenation product 3a″ as the substrate, the desired vinyl C—H olefination product 3a could be obtained in 38% yield, but only in the presence of optimal ligand L9. Taken together, these results suggest that the bidentate oxime ether-pyridone ligand L9 was responsible for both the β,γ-dehydrogenation and the vinyl C—H olefination pathways.

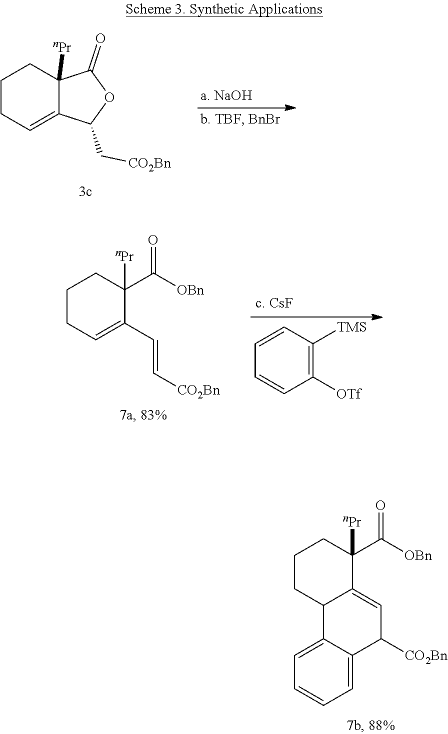

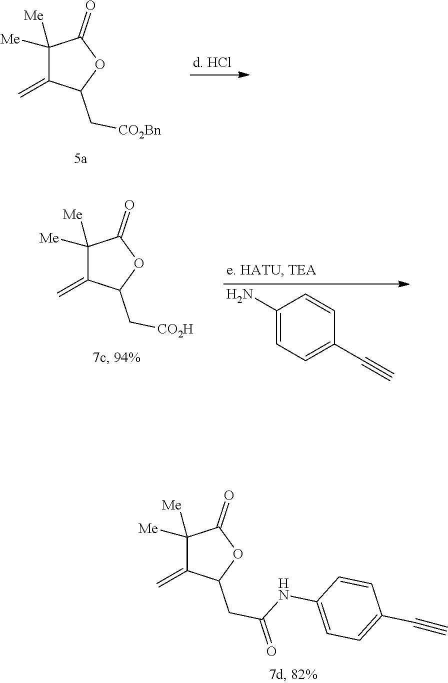

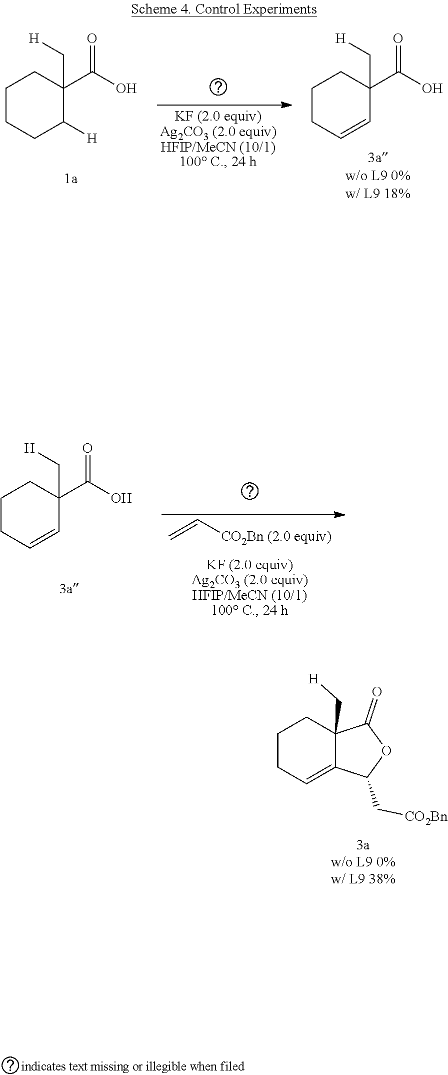

Based on the above control experiments and reported dehydrogenation reactions via β-C—H activation,^{3,4} we propose that our transformation proceeds via a Pd^{II}/Pd⁰ catalytic cycle outlined in Scheme 5. First, coordination of Pd(OAc)₂ to a bidentate oxime ether-pyridone or morpholine-pyridone ligand generates the active LPd^{II}(OAc) species. After coordination of the model substrate 1a to Pd to form int-I, both the countercation K⁺ and the pyridone ligand accelerate the selective cyclopalladation of the β-methylene C—H bond to form int-II. Next, β-hydride elimination from palladacycle int-II delivers β,γ-dehydrogenation product int-III, which is a possible catalyst inhibitor. After the regeneration of the Pd^{II} species by a Ag^{I} oxidant, bidentate pyridone ligand-promoted C(sp²)-H activation of int-IV generates vinyl palladacycle int-V. This intermediate can be subsequently coupled with acrylate through olefin insertion to yield int-VI. Finally, the diastereoselective Michael addition of int-VI delivers the fused γ-lactone product 3a, with reoxidation of Pd⁰ by Ag^{I} closing the catalytic cycle.

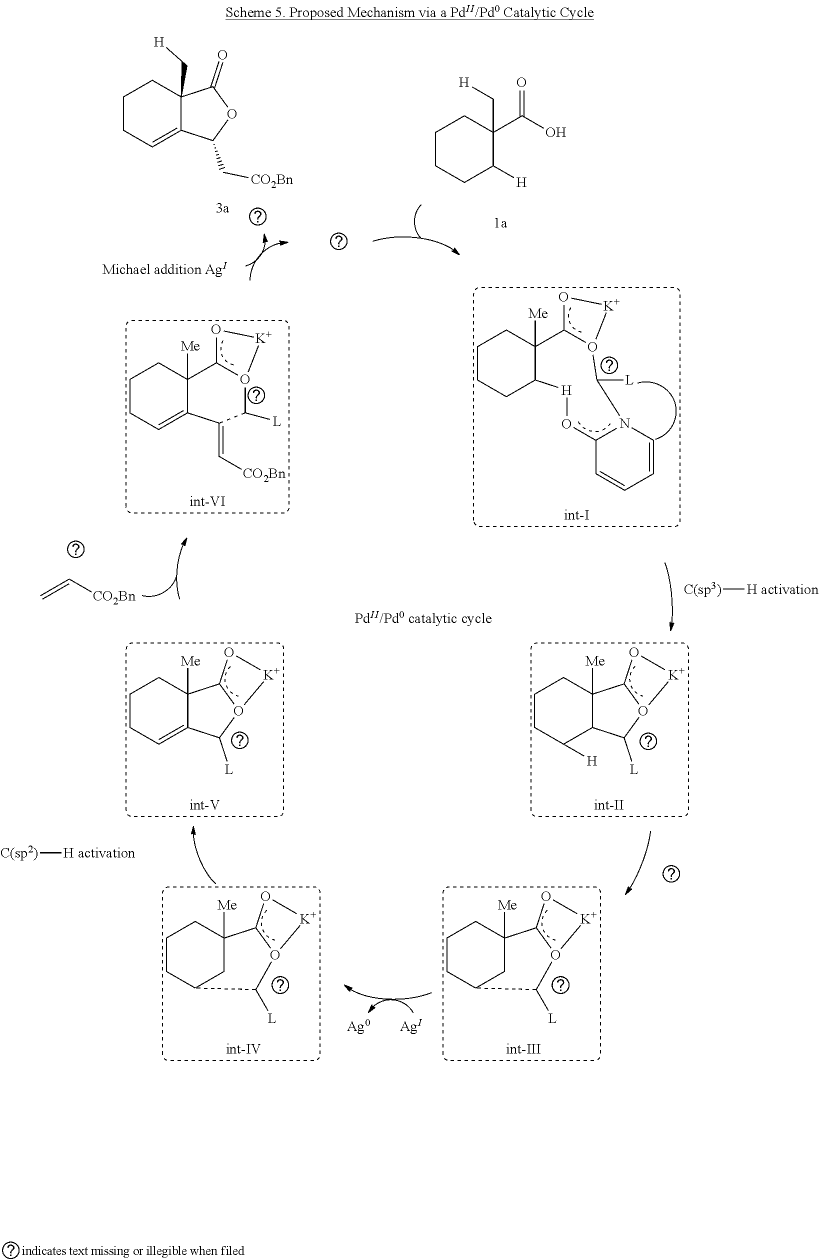

In effect, the above evidences the realization of an effective tandem reaction permitting the sequential β,γ-dehydrogenation and vinyl C—H olefination reactions of ubiquitous free aliphatic acids, including natural products such as isosteviol and grandiflorolic acid. This protocol affords a method for the one-step synthesis of a wide range of β-alkylidene-γ-lactones. Two new classes of pyridone-based ligands, oxime ether-pyridone and morpholine-pyridone, have been developed to enable an initial methylene-selective C—H activation and subsequent vinyl C—H activation.

### REFERENCES

1. Gnaim, S.; Vantourout, J. C.; Serpier, F.; Echeverria, P. G.; Baran, P. S. Carbonyl desaturation: where does catalysis stand? ACS Catal. 2021, 11, 883-892.
2. Zhao, Y.; Chen, Y.; Newhouse, T. R. Allyl-palladium catalyzed α,β-dehydrogenation of carboxylic acids via enediolates. Angew. Chem., Int. Ed. 2017, 56, 13122-13125.
3. Wang, Z.; Hu, L.; Chekshin, N.; Zhuang, Z.; Qian, S.; Qiao, J. X.; Yu, J.-Q. Ligand-controlled divergent dehydrogenative reactions of carboxylic acids via C—H activation. Science 2021, 374, 1281-1285.
4. Giri, R.; Maugel, N.; Foxman, B. M.; Yu, J.-Q. Dehydrogenation of inert alkyl groups via remote C—H activation: converting a propyl group into a x-allylic complex. Organometallics 2008, 27, 1667-1670.
5. For C(sp³)-H functionalization reactions of free carboxylic acids, see: (a) Giri, R.; Maugel, N.; Li, J.-J.; Wang, D.-H.; Breazzano, S. P.; Saunder, L. B.; Yu, J.-Q. Palladium-catalyzed methylation and arylation of sp² and sp³ C—H bonds in simple carboxylic acids. J. Am. Chem. Soc. 2007, 129, 3510-3511. (b) Chen, G.; Zhuang, Z.; Li, G.-C; Saint-Denis, T. G.; Hsiao, Y.; Joe, C. L.; Yu, J.-Q. Ligand-enabled β-C—H arylation of α-amino acids without installing exogenous directing groups. Angew. Chem., Int. Ed. 2017, 56, 1506-1509. (c) Zhu, Y.; Chen, X.; Yuan, C.; Li, G.; Zhang, J.; Zhao, Y. Pd-catalysed ligand-enabled carboxylate-directed highly regioselective arylation of aliphatic acids. Nat. Commun. 2017, 8, 14904. (d) Ghosh, K. K.; van Gemmeren, M. Pd-catalyzed β-C(sp³)-H arylation of propionic acid and related aliphatic acids. Chem.-Eur. J. 2017, 23, 17697-17700. (e) Shen, P.-X.; Hu, L.; Shao, Q.; Hong, K.; Yu, J.-Q. Pd(II)-catalyzed enantioselective C(sp³)-H arylation of free carboxylic acids. J. Am. Chem. Soc. 2018, 140, 6545-6549. (f) Zhuang, Z.; Yu, C.-B.; Chen, G.; Wu, Q.-F.; Hsiao, Y.; Joe, C. L.; Qiao, J. X.; Poss, M. A.; Yu, J.-Q. Ligand-enabled β-C(sp³)-H olefination of free carboxylic acids. J. Am. Chem. Soc. 2018, 140, 10363-10367. (g) Hu, L.; Shen, P.-X.; Shao, Q.; Hong, K.; Qiao, J. X.; Yu, J.-Q. Pd^{II}-catalyzed enantioselective C(sp³)-H activation/cross-coupling reactions of free carboxylic acids. Angew. Chem., Int. Ed. 2019, 58, 2134-2138. (h) Ghosh, K. K.; Uttry, A.; Koldemir, A.; Ong, M.; van Gemmeren, M. Direct β-C(sp³)-H acetoxylation of aliphatic carboxylic acids. Org. Lett. 2019, 21, 7154-7157. (i) Zhuang, Z.; Yu, J.-Q. Lactonization as a general route to β-C(sp³)-H functionalization. Nature 2020, 577, 656-659. (j) Zhuang, Z.; Herron, A. N.; Fan, Z.; Yu, J.-Q. Ligand-enabled monoselective β-C(sp³)-H acyloxylation of free carboxylic acids using a practical oxidant. J. Am. Chem. Soc. 2020, 142, 6769-6776. (k) Ghiringhelli, F.; Uttry, A.; Ghosh, K. K.; van Gemmeren, M. Direct β- and γ-C(sp³)-H alkynylation of free carboxylic acids. Angew. Chem., Int. Ed. 2020, 59, 23127-23131. (1) Zhuang, Z.; Herron, A. N.; Liu, S.; Yu, J.-Q. Rapid construction of tetralin, chromane, and indane motifs via cyclative C—H/C—H coupling: four-step total synthesis of (+)-russujaponol F. J. Am. Chem. Soc. 2021, 143, 687-692. (m) Zhuang, Z.; Herron, A. N.; Yu, J.-Q. Syntheses of cyclic anhydrides via ligand-enabled C—H carbonylation of simple aliphatic acids. Angew. Chem., Int. Ed. 2021, 60, 16382-16387. (n) Uttry, A.; Mal, S.; van Gemmeren, M. Late-stage β-C(sp³)-H deuteration of carboxylic acids. J. Am. Chem. Soc. 2021, 143, 10895-10901. For a review, see: (o) Lucas, E. L.; Lam, N. Y. S.; Zhuang, Z.; Chan, H. S. S.; Strassfeld, D. A.; Yu, J.-Q. Palladium-catalyzed enantioselective β-C(sp³)-H activation reactions of aliphatic acids: a retrosynthetic surrogate for enolate alkylation and conjugate addition. Acc. Chem. Res. 2022, 55, 537-550.
6. For reviews, see: (a) Chen, X.; Engle, K. M.; Wang, D.-H.; Yu, J.-Q. Palladium (II)-catalyzed C—H activation/C—C cross-coupling reactions: versatility and practicality. Angew. Chem., Int. Ed. 2009, 48, 5094-5115. (b) Daugulis, O.; Roane, J.; Tran, L. D. Bidentate, monoanionic auxiliary-directed functionalization of carbon-hydrogen bonds. Acc. Chem. Res. 2015, 48, 1053-1064. (c) Lyons, T. W.; Sanford, M. S. Palladium-catalyzed ligand-directed C—H functionalization reactions. Chem. Rev. 2010, 110, 1147-1169.
7. Provencher, P. A.; Hoskin, J. F.; Wong, J. J.; Chen, X.; Yu, J.-Q.; Houk, K. N.; Sorensen, E. J. Pd(II)-catalyzed synthesis of benzocyclobutenes by β-methylene-selective C(sp³)-H arylation with a transient directing group. J. Am. Chem. Soc. 2021, 143, 20035-20041.
8. For reviews, see: (a) He, J.; Wasa, M.; Chan, K. S. L.; Shao, Q.; Yu, J.-Q. Palladium-catalyzed transformations of alkyl C—H bonds. Chem. Rev. 2017, 117, 8754-8786. (b) Shao, Q.; Wu, K.; Zhuang, Z.; Qian, S.; Yu, J.-Q. From Pd(OAc)₂ to chiral catalysts: the discovery and development of bifunctional mono-N-protected amino acid ligands for diverse C—H activation reactions. Acc. Chem. Res. 2020, 53, 833-851.
9. (a) He, J.; Li, S.; Deng, Y.; Fu, H.; Laforteza, B. N.; Spangler, J. E.; Homs, A.; Yu, J.-Q. Ligand-controlled C(sp³)-H arylation and olefination in synthesis of unnatural chiral α-amino acids. Science 2014, 343, 1216-1220. (b) Chen, Y.-Q.; Wang, Z.; Wu, Y.; Wisniewski, S. R.; Qiao, J. X.; Ewing, W. R.; Eastgate, M. D.; Yu, J.-Q. Overcoming the limitations of γ- and δ-C—H arylation of amines through ligand development. J. Am. Chem. Soc. 2018, 140, 17884-17894.
10. Li, Z.; Wang, Z.; Chekshin, N.; Qian, S.; Qiao, J. X.; Cheng, P. T.; Yeung, K. S.; Ewing, W. R.; Yu, J.-Q. A tautomeric ligand enables directed C—H hydroxylation with molecular oxygen. Science 2021, 372, 1452-1457.
11. (a) Baldwin, J. E.; Jones, R. H.; Najera, C.; Yus, M. Functionalisation of unactivated methyl groups through cyclopalladation reactions. Tetrahedron 1985, 41, 699-711. (b) Desai, L. V.; Hull, K. L.; Sanford, M. S. Palladium-catalyzed oxygenation of unactivated sp³ C—H Bonds. J. Am. Chem. Soc. 2004, 126, 9542-9543.
12. (a) Ullah, A.; Munir, S.; Mabkhot, Y.; Badshah, S. L. Bioactivity profile of the diterpene isosteviol and its derivatives. Molecules 2019, 24, 678. (b) Piozzi, F.; Sprio, V.; Passannanti, S.; Mondelli, R. Structura dell'acido gradiflorolico. Gazz. Chim. Ital. 1968, 98, 907-910.
13. (a) Wenkert, E.; Alonso, M. E.; Buckwalter, B. L.; Chou, K. J. A method of synthesis of β-methylfurans and α-methylene and β-methylene γ-lactones. Two menthofuran syntheses. J. Am. Chem. Soc. 1977, 99, 4778-4782. (b) Greene, A. E.; Coelho, F.; Depres, J.-P. A synthesis of β-methylene-γ-butyrolactones. J. Org. Chem. 1985, 50, 1973-1975.
14. Wang, Y.-C.; Huang, Y.-H.; Tsai, H.-C.; Basha, R. S.; Chou, C.-M. Palladium-catalyzed proaromatic C(alkenyl)-H olefination: synthesis of densely functionalized 1,3-dienes. Org. Lett. 2020, 22, 6765-6770.

### EMBODIMENTS

The application provides the following embodiments:

Embodiment 1. A method of forming a β-alkylidene-γ-lactone, comprising treating an α-substituted carboxylic acid with a vinyl reagent containing an electron withdrawing group (EWG) in the presence of a Pd source/Ligand (L) catalyst.

Embodiment 2. The method of Embodiment 1, wherein the method of forming a β-alkylidene-γ-lactone occurs according to the following reaction scheme:

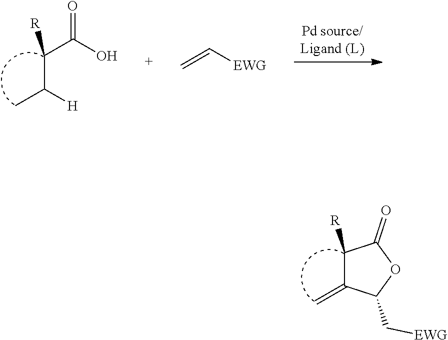

wherein

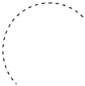

represents a substituted or unsubstituted C₅-C₁₂ ring system; and
R is optionally substituted —(C₁-C₆)alkyl, aryl, —(C₁-C₆) haloalkyl, —O(C₁-C₆)alkyl, cyclocalkyl, —(C₁-C₆)alkylcycloalkyl, or —(C₁-C₆)alkylaryl.

Embodiment 3. The method of either Embodiment 1 or Embodiment 2, wherein the method of forming a β-alkylidene-γ-lactone occurs according to the following reaction scheme:

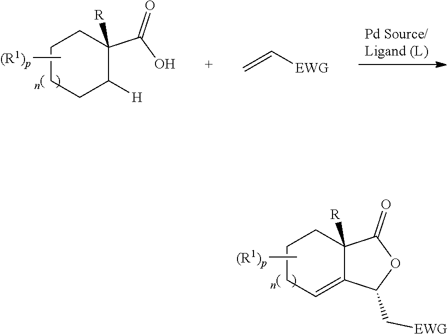

wherein:
R is optionally substituted —(C₁-C₆)alkyl, aryl, —(C₁-C₆) haloalkyl, —O(C₁-C₆)alkyl, cyclocalkyl, —(C₁-C₆)alkylcycloalkyl, or —(C₁-C₆)alkylaryl;
EWG is —CO₂Bn, —CN, CO₂R′, —C(═O)R′, —SO₂R′, or —P(O)(OR′)₂;
   each R′ is independently optionally substituted —(C₁-C₁₂)alkyl, —O(C₁-C₁₂)alkyl,
   cycloalkyl, heterocycloalkyl, —N((C₁-C₆)alkyl)₂, or Ph;
each R¹ is independently optionally substituted —(C₁-C₆)alkyl or —O(C₁-C₆)alkyl;
n is 0-7; and
p is 0-4.

Embodiment 4. The method of Embodiment 3, wherein n is 1; and p is 0 or 1.

Embodiment 5. The method of Embodiment 3, wherein n is 1 or 2; and p is 0, 1, or 2.

Embodiment 6. The method of Embodiment 3, wherein n is 1, and p is 0, 1, or 2.

Embodiment 7. The method of Embodiment 3, wherein n is 1; and p is 0.

Embodiment 8. The method of Embodiment 3, wherein n is 1; and p is 1.

Embodiment 9. The method of Embodiment 3, wherein n is 1; and p is 2.

Embodiment 10. The method of Embodiment 3, wherein n is 1; and p is 0.

Embodiment 11. The method of Embodiment 3, wherein n is 1, 2, or 3; and p is 0.

Embodiment 12. The method of any one of Embodiments 1-11, wherein the Ligand (L) is selected from the group consisting of:

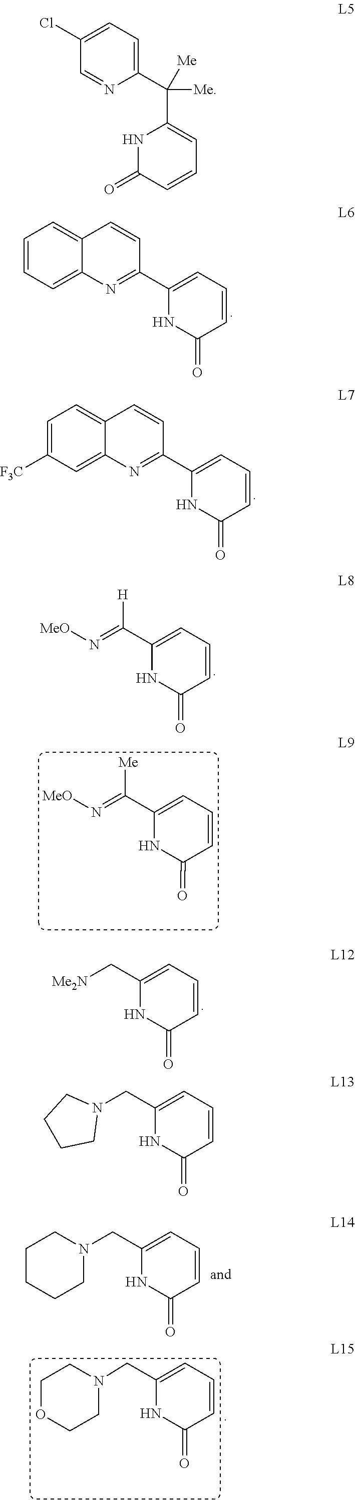

Embodiment 13. The method of any one of Embodiments 1-12, wherein the Ligand (L) is L6 or L9, or L15.

Embodiment 14. The method of any one of Embodiments 1-13, wherein the Ligand (L) is L9 or L15.

Embodiment 15. The method of any one of Embodiments 1-13, wherein the Ligand (L) is L6.

Embodiment 16. The method of any one of Embodiments 1-14, wherein the Ligand (L) is L9.

Embodiment 17. The method of any one of Embodiments 1-14, wherein the Ligand (L) is L15.

Embodiment 18. The method of any one of Embodiments 1-17, wherein the Ligand (L) is present in approximately 10-20 mol %.

Embodiment 19. The method of any one of Embodiments 1-18, wherein the Ligand (L) is present in approximately 13 mol %.

Embodiment 20. The method of any one of Embodiments 1-19, wherein the Pd source is a Pd salt.

Embodiment 21. The method of any one of Embodiments 1-20, wherein the Pd source is Pd(OAc)₂, PdCl₂C(CH₃CN)₂, Pd(TFA)₂, Pd(dba)₂, Pd(PhCN)₂Cl₂, or Pd(CH₃CN)₄(OTf)₂.

Embodiment 22. The method of any one of Embodiments 1-21, wherein the Pd source is Pd(OAc)₂.

Embodiment 23. The method of any one of Embodiments 1-21, wherein the Pd source is PdCl₂C(CH₃CN)₂.

Embodiment 24. The method of any one of Embodiments 1-21, wherein the Pd source is Pd(TFA)₂.

Embodiment 25. The method of any one of Embodiments 1-21, wherein the Pd source is Pd(dba)₂.

Embodiment 26. The method of any one of Embodiments 1-21, wherein the Pd source is Pd(PhCN)₂Cl₂.

Embodiment 27. The method of any one of Embodiments 1-21, wherein the Pd source is Pd(CH₃CN)₄(OTf)₂.

Embodiment 28. The method of any one of Embodiments 1-27, wherein the Pd source is present in approximately 5-20 mol %.

Embodiment 29. The method of any one of Embodiments 1-28, wherein the Pd source is present in approximately 10 mol %.

Embodiment 30. The method of any one of Embodiments 1-29, wherein the method of forming a β-alkylidene-γ-lactone occurs in the presence of an oxidant.

Embodiment 31. The method of Embodiment 30, wherein the oxidant is an Ag salt.

Embodiment 32. The method of Embodiment 31, wherein the Ag salt is Ag₂CO₃, AgOAc, AgF, AgTFA, Ag₂O, or Ag₃PO₄.

Embodiment 33. The method of Embodiment 32, wherein the Ag salt is Ag₂CO₃.

Embodiment 34. The method of Embodiment 32, wherein the Ag salt is AgOAc.

Embodiment 35. The method of Embodiment 32, wherein the Ag salt is AgF.

Embodiment 36. The method of Embodiment 32, wherein the Ag salt is AgTFA.

Embodiment 37. The method of Embodiment 32, wherein the Ag salt is Ag₂₀.

Embodiment 38. The method of any one of Embodiments 30-37, wherein the oxidant is present in approximately 1.0-3.0 equivalents.

Embodiment 39. The method of any one of Embodiments 30-38, wherein the oxidant is present in approximately 2.0 equivalents.

Embodiment 40. The method of any one of Embodiments 1-39, wherein the method of forming a β-alkylidene-γ-lactone occurs in the presence of an HFIP solvent mixture.

Embodiment 41. The method of Embodiment 40, wherein the HFIP solvent mixture is approximately 10:1 HFIP:MeCN.

Embodiment 42. The method of Embodiment 40, wherein the HFIP solvent mixture is approximately 10:1.5 HFIP:MeCN.

Embodiment 43. The method of Embodiment 40, wherein the HFIP solvent mixture is approximately 10:1 HFIP:CH₃ (CH₂)₂CN.

Embodiment 44. The method of Embodiment 40, wherein the HFIP solvent mixture is approximately 20:1 HFIP:MeCN.

Embodiment 45. The method of Embodiment 40, wherein the HFIP solvent mixture is approximately 10:1 HFIP:DMSO.

Embodiment 46. The method of any one of Embodiments 1-45, wherein the method of forming a β-alkylidene-γ-lactone occurs according to the following reaction scheme:

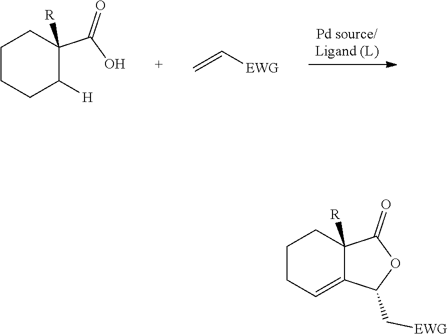

wherein R is optionally substituted —(C₁-C₆)alkyl, aryl, —(C₁-C₆) haloalkyl, —O(C₁-C₆)alkyl, cyclocalkyl, —(C₁-C₆)alkylcycloalkyl, or —(C₁-C₆)alkylaryl.

Embodiment 47. The method of Embodiment 46, wherein R is Me.

Embodiment 48. The method of Embodiment 46, wherein R is iPr.

Embodiment 49. The method of any one of Embodiments 1-2, 15, 18-22, 28-33, or 38-41, wherein the method of forming a β-alkylidene-γ-lactone occurs according to the following reaction scheme:

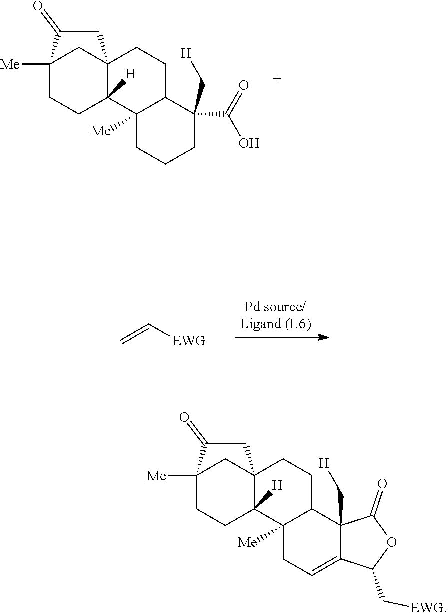

Embodiment 50. The method of any one of Embodiments 1-2, 15, 18-22, 28-33, or 38-41, wherein the method of forming a β-alkylidene-γ-lactone occurs according to the following reaction scheme:

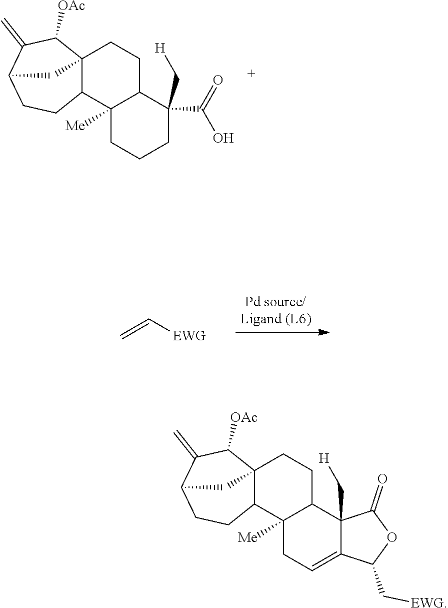

Embodiment 51. The method of any one of Embodiments 1-50, wherein EWG is —CO₂Bn.

Embodiment 52. The method of any one of Embodiments 1-13, 18-22, 28-33, or 38-41, wherein the β-alkylidene-γ-lactone is selected from the group consisting of Formulae (3a-3aa):

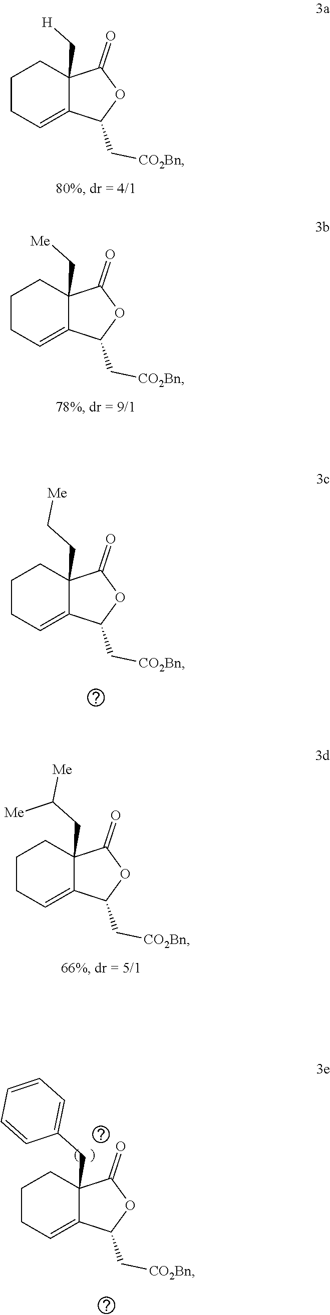

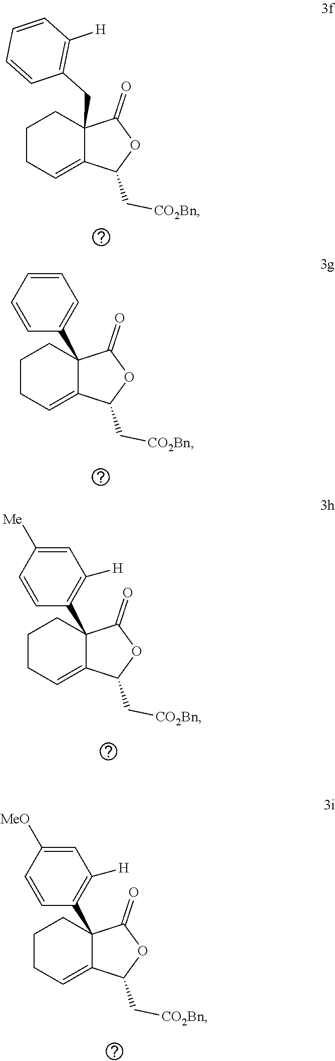 [SMILES: O=C(C[C@H]1OC(=O)[C@@]2(Cc3ccccc3)CCCC=C12)OCc1ccccc1 ; O=C(C[C@H]1OC(=O)[C@@]2(c3ccccc3)CCCC=C12)OCc1ccccc1 ; Cc1ccc([C@]23CCCC=C2[C@@H](CC(=O)OCc2ccccc2)OC3=O)cc1 ; COc1ccc([C@]23CCCC=C2[C@@H](CC(=O)OCc2ccccc2)OC3=O)cc1]

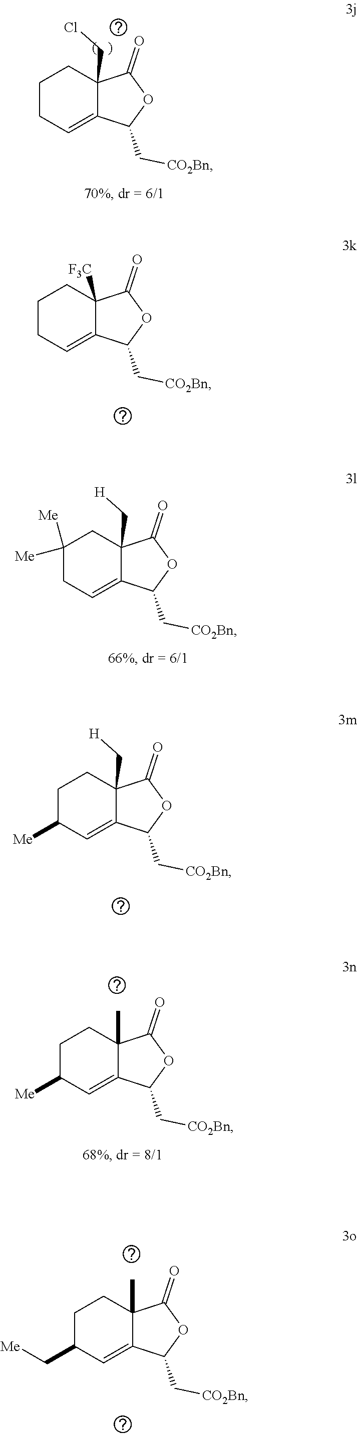

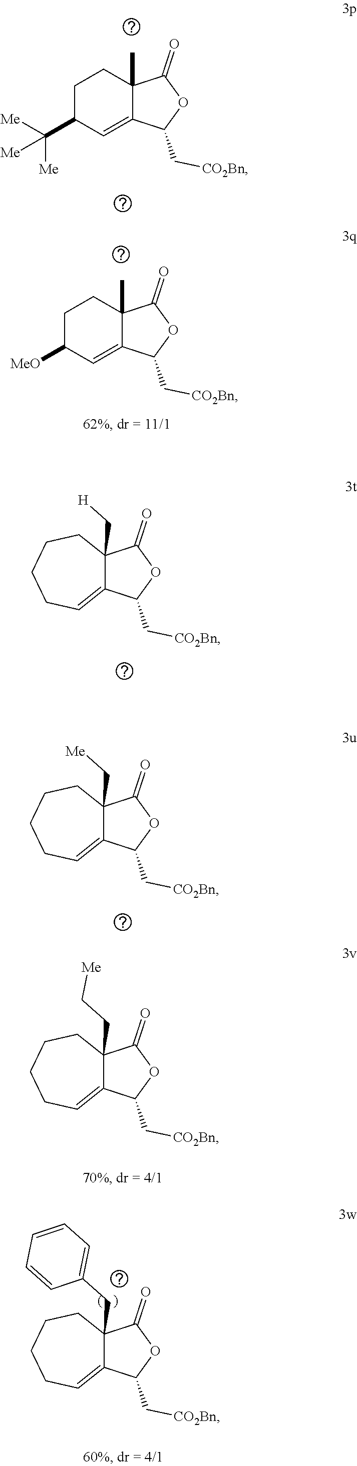

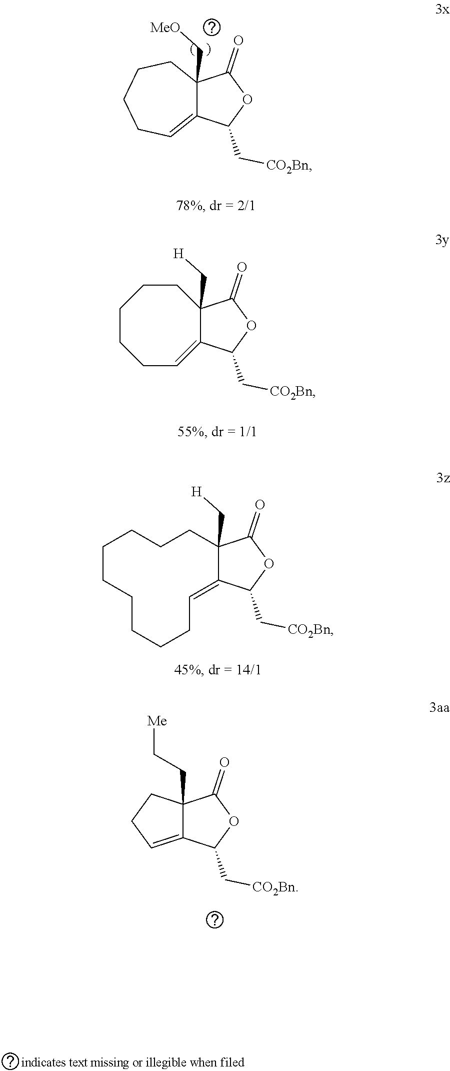

Embodiment 53. The method of Embodiment 1, wherein the method of forming a β-alkylidene-γ-lactone occurs according to the following reaction:

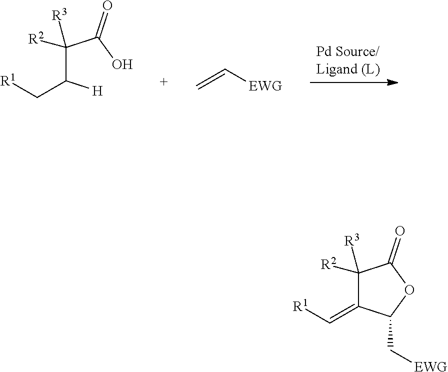

wherein:
R¹ is H, —(C₁-C₆)alkyl, or —(C₁-C₆)alkylO(C₁-C₆)alkyl;
R² and R³ are independently —(C₁-C₆)alkyl, —(C₁-C₆)alkylPh, —(C₁-C₆)alkylcycloalkyl, —(C₁-C₆)alkylO(C₁-C₆)alkyl, or halo(C₁-C₆)alkyl;
EWG is —CO₂Bn, CN, —CO₂R′, —C(═O)R′, —SO₂R′, or —P(O)(OR′)₂; and
   each R′ is independently optionally substituted —(C₁-C₁₂)alkyl, —O(C₁-C₁₂)alkyl, cycloalkyl, heterocycloalkyl, —N((C₁-C₆)alkyl)₂, or Ph.

Embodiment 54. The method of Embodiment 53, wherein the method of forming a β-alkylidene-γ-lactone occurs in the presence of NaOAc.

Embodiment 55. The method of Embodiment 54, wherein the NaOAc is present in approximately 2.0 equivalents.

Embodiment 56. The method of any one of Embodiments 53-55, wherein EWG is CO₂Bn.

Embodiment 57. The method of Embodiment 53, wherein the a-substituted aliphatic carboxylic acid (1.0 eq.) is treated with benzyl acrylate (2.0 eq.) in the presence of Pd(OAc)₂ (10 mol %), L9 (13 mol %), NaOAc (2.0 eq.), and Ag₂CO₃ (2.0 eq.), in HFIP/MeCN (19/1).

Embodiment 58. The method of Embodiment 1, wherein the method of forming a β-alkylidene-γ-lactone occurs according to the following reaction scheme:

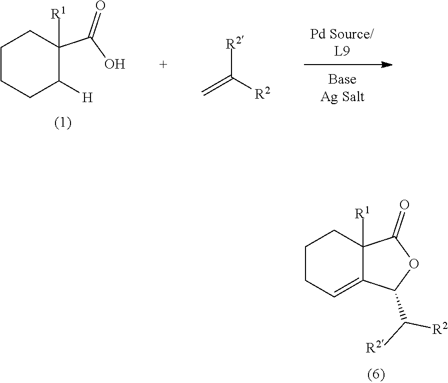

wherein:
R¹ is optionally substituted —(C₁-C₆)alkyl, aryl, —(C₁-C₆) haloalkyl, —O(C₁-C₆)alkyl, cyclocalkyl, —(C₁-C₆)alkylcycloalkyl, or —(C₁-C₆)alkylaryl;
R² is H or —CH₂CO₂ (C₁-C₆)alkyl;
R^{2′} is —CO₂Rₐ, CN, —C(═O)R_{b}, —S(═O)₂ (C₁-C₆)alkyl, —S(═O)₂Ph, —S(═O)₂N(C₁-C₆alkyl)₂, or —P(═O)(O(C₁-C₆)alkyl)₂;
   Rₐ is —(C₁-C₁₂)alkyl, cycloalkyl, or —(C₁-C₆)alkyl-O—(C₁-C₆)alkyl; and
   R_{b} is —N(C₁-C₆ alkyl)₂ or heterocycloalkyl.

Embodiment 59. The method of Embodiment 58, wherein the Pd source is Pd(OAc)₂.

Embodiment 60. The method of either Embodiment 58 or Embodiment 59, wherein the Ag salt is Ag₂CO₃.

Embodiment 61. The method of any one of Embodiments 58-60, wherein the Base is KF.

Embodiment 62. The method of any one of Embodiments 58-60, wherein the Base is LiF.

Embodiment 63. The method of any one of Embodiments 58-62, wherein the solvent mixture is approximately 10:1.0 HFIP:MeCN.

Embodiment 64. The method of any one of Embodiments 58-62, wherein the solvent mixture is approximately 10:1.5 HFIP:MeCN.

Embodiment 65. The method of any one of Embodiments 58-64, wherein R² is H.

Embodiment 66. The method of Embodiment 58, wherein the a-substituted aliphatic carboxylic acid substrate (1.0 eq.) is treated with CH₂C(R²)(R^{2′})(2.0 eq.) in the presence of Pd(OAc)₂ (10 mol %), L9 (13 mol %), Base (2.0 eq.), and Ag₂CO₃ (2.0 eq.), in HFIP/MeCN.

Embodiment 67. A method of the synthesis of the compound of Formula (7b) from the γ-lactone product of Formula (3c) prepared according to the method of Embodiment 48, comprising the steps of:

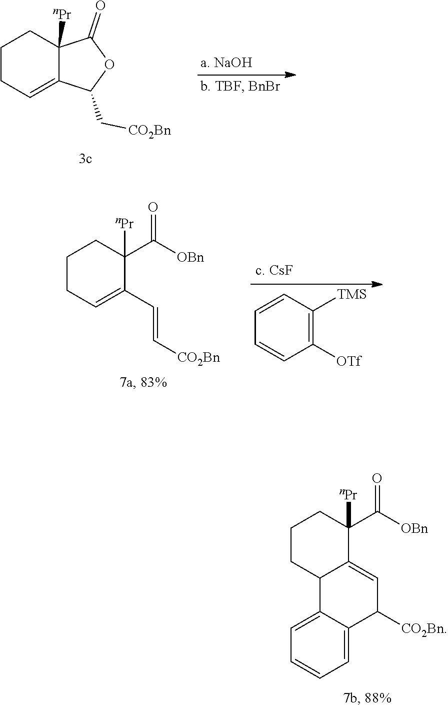

Embodiment 68. A method of the synthesis of Formula (7d) from the γ-lactone product of Formula (5a) prepared according to the method of Embodiment 53, comprising the steps of:

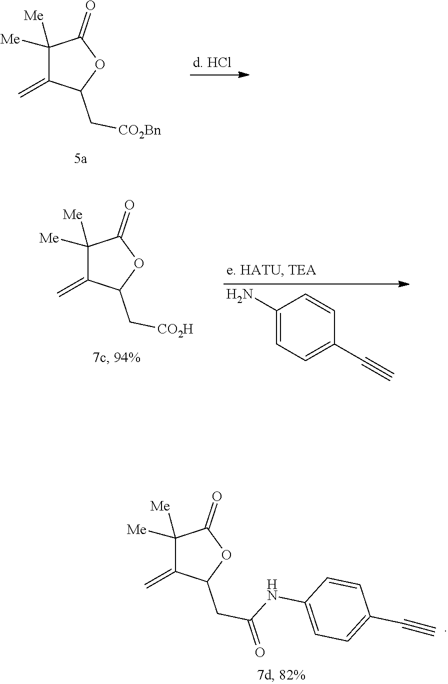

Embodiment 69. Any method of synthesis of β-alkylidene-γ-lactones via dehydrogenation-olefination-lactonization of free aliphatic carboxylic acid substrates, including natural products isosteviol and grandiflorolic acid, or method of further synthesis involving the product β-alkylidene-γ-lactones as disclosed herein.

### Definitions

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention, the Detailed Description of the Invention, the Experimentals, or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

As used herein, unless specifically indicated otherwise, the word “or” is used in the “inclusive” sense of “and/or” and not the “exclusive” sense of “either/or”.

The term “independently” is used herein to indicate that a variable is applied in any one instance without regard to the presence or absence of a variable having that same or a different definition within the same compound. Thus, in a compound in which “R” appears twice and is defined as “independently selected from” means that each instance of that R group is separately identified as one member of the set which follows in the definition of that R group. For example, “each R¹ and R² is independently selected from carbon and nitrogen” means that both R¹ and R² can be carbon, both R¹ and R² can be nitrogen, or R¹ or R² can be carbon and the other nitrogen or vice versa.

When any variable occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or a line drawn through a bond or “ [symbol] ” drawn through a bond each refer to the point of attachment of a functional group or other chemical moiety to the rest of the molecule of which it is a part.

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the “optionally substituted” moiety may incorporate a hydrogen or a substituent.

The phrase “optional bond” means that the bond may or may not be present, and that the description includes single, double, or triple bonds. If a substituent is designated to be a “bond” or “absent”, the atoms linked to the substituents are then directly connected.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Certain compounds disclosed herein may exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—□—C(—OH)═CH—), amide/imidic acid (—C(═O)—NH—□—C(—OH)═N—) and amidine (—C(═NR)—NH—□—C(—NHR)═N—) tautomers. The latter two are particularly common in heteroaryl and heterocyclic rings and the present invention encompasses all tautomeric forms of the compounds.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

The definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” and the like. When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero) aryl or (het) aryl refers to either an aryl or a heteroaryl group.

The term “acyl” as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term C₁₋₆ acyl refers to a group —C(═O)R contain 6 carbon atoms. The term “arylcarbonyl” as used herein means a group of formula C(—O)R wherein R is an aryl group; the term “benzoyl” as used herein an “arylcarbonyl” group wherein R is phenyl.

The term “alkyl” as used herein denotes an unbranched or branched chain, saturated, monovalent hydrocarbon residue containing 1 to 12 carbon atoms. The term “lower alkyl” or “C₁-C₆ alkyl” as used herein denotes a straight or branched chain hydrocarbon residue containing 1 to 6 carbon atoms. “C₁₋₁₂ alkyl” as used herein refers to an alkyl composed of 1 to 12 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” denotes the radical R′R″—, wherein R′ is a phenyl radical, and R″ is an alkylene radical as defined herein with the understanding that the attachment point of the phenylalkyl moiety will be on the alkylene radical. Examples of arylalkyl radicals include, but are not limited to, benzyl, phenylethyl, 3-phenylpropyl. The terms “arylalkyl” or “aralkyl” are interpreted similarly except R′ is an aryl radical. The terms “(het) arylalkyl” or “(het) aralkyl” are interpreted similarly except R′ is optionally an aryl or a heteroaryl radical.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 15 carbon atoms (“C₁₋₁₅ alkyl”). In some embodiments, an alkyl group has 1 to 14 carbon atoms (“C₁₋₁₄ alkyl”). In some embodiments, an alkyl group has 1 to 13 carbon atoms (“C₁₋₁₃ alkyl”). In some embodiments, an alkyl group has 1 to 12 carbon atoms (“C₁₋₁₂ alkyl”). In some embodiments, an alkyl group has 1 to 11 carbon atoms (“C₁₋₁₁ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like.

“Alkenyl” or “olefin” refers to a radical of a straight-chain or branched hydrocarbon group having from 2 to 10 carbon atoms and 1, 2, 3, or 4 carbon-carbon double bonds (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

“Alkynyl” refers to a radical of a straight-chain or branched hydrocarbon group having from 2 to 10 carbon atoms and one or more carbon-carbon triple bonds (e.g., 1, 2, 3, or 4 triple bonds)(“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

The terms “haloalkyl” or “halo-lower alkyl” or “lower haloalkyl” refers to a straight or branched chain hydrocarbon residue containing 1 to 6 carbon atoms wherein one or more carbon atoms are substituted with one or more halogen atoms.

The term “alkylene” or “alkylenyl” as used herein denotes a divalent saturated linear hydrocarbon radical of 1 to 10 carbon atoms (e.g., (CH₂) n) or a branched saturated divalent hydrocarbon radical of 2 to 10 carbon atoms (e.g., —CHMe- or —CH₂CH(i-Pr)CH₂—), unless otherwise indicated. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, 2-ethylbutylene.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an —O-alkyl wherein alkyl is C₁₋₁₀.

The term “hydroxyalkyl” as used herein denotes an alkyl radical as herein defined wherein one to three hydrogen atoms on different carbon atoms is/are replaced by hydroxyl groups.

The terms “alkylsulfonyl” and “arylsulfonyl” as used herein refers to a group of formula —S(═O)₂R wherein R is alkyl or aryl respectively and alkyl and aryl are as defined herein. The term “heteroalkylsulfonyl” as used herein refers herein denotes a group of formula —S(═O)₂R wherein R is “heteroalkyl” as defined herein.

The terms “alkylsulfonylamino” and “arylsulfonylamino” as used herein refers to a group of formula —NR′S(—O)₂R wherein R is alkyl or aryl respectively, R′ is hydrogen or C₁₋₃ alkyl, and alkyl and aryl are as defined herein.

The term “cycloalkyl” as used herein refers to a saturated carbocyclic ring containing 3 to 8 carbon atoms, i.e. cyclopropyl, cyclobutyl, cyclopentyl, cyclohexyl, cycloheptyl or cyclooctyl. “C₃₋₇ cycloalkyl” as used herein refers to an cycloalkyl composed of 3 to 7 carbons in the carbocyclic ring.

The term carboxy-alkyl as used herein refers to an alkyl moiety wherein one, hydrogen atom has been replaced with a carboxyl with the understanding that the point of attachment of the heteroalkyl radical is through a carbon atom. The term “carboxy” or “carboxyl” refers to a —CO₂H moiety.

The term “heteroaryl” or “heteroaromatic” as used herein means a monocyclic or bicyclic radical of 5 to 12 ring atoms having at least one aromatic ring containing four to eight atoms per ring, incorporating one or more N, O, or S heteroatoms, the remaining ring atoms being carbon, with the understanding that the attachment point of the heteroaryl radical will be on an aromatic ring. As well known to those skilled in the art, heteroaryl rings have less aromatic character than their all-carbon counter parts. Thus, for the purposes of the invention, a heteroaryl group need only have some degree of aromatic character. Examples of heteroaryl moieties include monocyclic aromatic heterocycles having 5 to 6 ring atoms and 1 to 3 heteroatoms include, but is not limited to, pyridinyl, pyrimidinyl, pyrazinyl, pyrrolyl, pyrazolyl, imidazolyl, oxazol, isoxazole, thiazole, isothiazole, triazoline, thiadiazole and oxadiaxoline which can optionally be substituted with one or more, preferably one or two substituents selected from hydroxy, cyano, alkyl, alkoxy, thio, lower haloalkoxy, alkylthio, halo, lower haloalkyl, alkylsulfinyl, alkylsulfonyl, halogen, amino, alkylamino, dialkylamino, aminoalkyl, alkylaminoalkyl, and dialkylaminoalkyl, nitro, alkoxycarbonyl and carbamoyl, alkylcarbamoyl, dialkylcarbamoyl, arylcarbamoyl, alkylcarbonylamino and arylcarbonylamino. Examples of bicyclic moieties include, but are not limited to, quinolinyl, isoquinolinyl, benzofuryl, benzothiophenyl, benzoxazole, benzisoxazole, benzothiazole and benzisothiazole. Bicyclic moieties can be optionally substituted on either ring; however the point of attachment is on a ring containing a heteroatom.

The term “heterocyclyl”, “heterocycloalkyl” or “heterocycle” as used herein denotes a monovalent saturated cyclic radical, consisting of one or more rings, preferably one to two rings, including spirocyclic ring systems, of three to eight atoms per ring, incorporating one or more ring heteroatoms (chosen from N, O or S(O)₀₋₂), and which can optionally be independently substituted with one or more, preferably one or two substituents selected from hydroxy, oxo, cyano, lower alkyl, lower alkoxy, lower haloalkoxy, alkylthio, halo, lower haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino, arylcarbonylamino, unless otherwise indicated. Examples of heterocyclic radicals include, but are not limited to, azetidinyl, pyrrolidinyl, hexahydroazepinyl, oxetanyl, tetrahydrofuranyl, tetrahydrothiophenyl, oxazolidinyl, thiazolidinyl, isoxazolidinyl, morpholinyl, piperazinyl, piperidinyl, tetrahydropyranyl, thiomorpholinyl, quinuclidinyl and imidazolinyl.

“Heterocyclyl” or “heterocyclic” refers to a group or radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆-14 aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl (α-naphthyl) and 2-naphthyl (β-naphthyl)). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

“Heteroaryl” refers to a radical of a 5-14 membered monocyclic or polycyclic (e.g., bicyclic, tricyclic) 4n+2 aromatic ring system (e.g., having 6, 10, or 14 pi electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Saturated” refers to a ring moiety that does not contain a double or triple bond, i.e., the ring contains all single bonds.

Alkyl, cycloalkyl, heterocyclyl, aryl, and heteroaryl groups may be optionally substituted. Optionally substituted refers to a group which may be substituted or unsubstituted. In general, the term “substituted” means that at least one hydrogen present on a group is replaced with a non-hydrogen substituent, and which upon substitution results in a stable compound, e.g., a compound which does not spontaneously undergo transformation such as by rearrangement, cyclization, elimination, or other reaction. Heteroatoms such as nitrogen, oxygen, and sulfur may have hydrogen substituents and/or non-hydrogen substituents which satisfy the valencies of the heteroatoms and results in the formation of a stable compound.

Exemplary non-hydrogen substituents wherein a moiety is “optionally substituted” as used herein means the moiety may be substituted with any additional moiety selected from, but not limited to, the group consisting of halogen, —CN, —NO₂, —N₃, —SO₂H, —SO₃H, —OH, —OR^{aa}, —N(R^{bb})₂, —N(OR^{cc})R^{bb}, —SH, —SR^{aa}, —C(═O)R^{aa}, —CO₂H, —CHO, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —S(═O)R^{aa}, —OS(═O)R^{aa}, —B(OR^{aa})₂, C₁₋₁₀ alkyl, C₂₋₁₀ alkenyl, C₂₋₁₀ alkynyl, C₃₋₁₄ carbocyclyl, 3- to 14-membered heterocyclyl, C₆-14 aryl, and 5- to 14-membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups, or two geminal hydrogens on a carbon atom are replaced with the group-O; each instance of R^{aa} is, independently, selected from the group consisting of C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆-14 aryl, and 5- to 14-membered heteroaryl, or two R^{aa} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{bb} is, independently, selected from the group consisting of hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —SO₂N(R^{cc})₂, —SOR^{aa}, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆-14 aryl, and 5- to 14-membered heteroaryl, or two R^{bb} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{cc} is, independently, selected from the group consisting of hydrogen, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆-14 aryl, and 5- to 14-membered heteroaryl, or two R^{cc} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; and each instance of R^{dd} is, independently, selected from the group consisting of halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(OC₁₋₆ alkyl)(C₁₋₆ alkyl), —N(OH)(C₁₋₆ alkyl), —NH(OH), —SH, —SC₁₋₆ alkyl, —C(═O)(C₁₋₆ alkyl), —CO₂H, —CO₂ (C₁₋₆ alkyl), —OC(═O)(C₁₋₆ alkyl), —OCO₂ (C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl) C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —B(OH)₂, —B(OC₁₋₆ alkyl)₂,C₁₋₆ alkyl, C₁₋₆ perhaloalkyl, C₂₋₆ alkenyl, C₂₋₆ alkynyl, C₃₋₁₀ carbocyclyl, C₆-10 aryl, 3- to 10-membered heterocyclyl, and 5- to 10-membered heteroaryl; or two geminal R^{dd} substituents on a carbon atom may be joined to form ═O.

“Halo” or “halogen” refers to fluorine (fluoro, —F), chlorine (chloro, —C₁), bromine (bromo, —Br), or iodine (iodo, —I).

As used herein, the term “composition” is intended to encompass a product comprising the specified ingredients, as well as any product which results, directly or indirectly, from combination of the specified ingredients.

“Salt” includes any and all salts. “Pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts include those derived from inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl) 4 salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Unless otherwise indicated, compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC). Compounds described herein can be in the form of individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, replacement of a carbon by a ¹³C- or ¹⁴C-enriched carbon, and/or replacement of an oxygen atom with ¹⁸O, are within the scope of the disclosure. Other examples of isotopes include ¹⁵N, ¹⁸O, ¹⁷O, ³¹P, ³²P, ³⁵S, ¹⁸F, ³⁶Cl and ¹²³I. Compounds with such isotopically enriched atoms are useful, for example, as analytical tools or probes in biological assays.

Certain isotopically-labelled compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability.

Certain isotopically-labelled compounds of Formula (I) can be useful for medical imaging purposes, for example, those labeled with positron-emitting isotopes like ¹¹C or ¹⁸F can be useful for application in Positron Emission Tomography (PET) and those labeled with gamma ray emitting isotopes like ¹²³I can be useful for application in Single Photon Emission Computed Tomography (SPECT). Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Further, substitution with heavier isotopes such as deuterium (i.e., ²H) may afford certain therapeutic advantages resulting from greater metabolic stability (e.g., increased in vivo half-life or reduced dosage requirements), and hence, may be preferred in some circumstances. Additionally, isotopic substitution at a site where epimerization occurs may slow or reduce the epimerization process and thereby retain the more active or efficacious form of the compound for a longer period of time. Isotopically labeled compounds of Formula (I), in particular those containing isotopes with longer half-lives (t_{1/2}>1 day), can generally be prepared by following procedures analogous to those disclosed in the Schemes and/or in the Examples herein below, by substituting an appropriate isotopically labeled reagent for a non-isotopically labeled reagent.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

### Methods

In some embodiments, the reaction conditions include the α-substituted carboxylic acid being approximately 0.1 mmol. In some embodiments, the reaction conditions include the vinyl reagent with an electron withdrawing group (EWG) being approximately 0.2 mmol. In some embodiments, the reaction conditions include the Ligand (L) being approximately 1.3 equivalents relative to the Pd source. In some embodiments, the reaction conditions include the Ligand (L) being approximately 13 mol %. In some exemplary embodiments, the Ligand L is L9. In some exemplary embodiments the Ligand L is L15. In some embodiments, the reaction conditions include the Pd source being approximately 10 mol %. In some exemplary embodiments, the Pd source is Pd(OAc)₂. In some embodiments, the reaction conditions include the silver salt oxidant being approximately 2.0 equivalents relative to the a-substituted carboxylic acid. In some exemplary embodiments, the silver salt is Ag₂CO₃. In some embodiments, the reaction conditions include the base being approximately 2.0 equivalents relative to the a-substituted carboxylic acid. In some exemplary embodiments, the base is KF. In some embodiments, the reaction conditions include the solvent being and HFIP mixture. In some exemplary embodiments, the solvent is 10:1 HFIP:MeCN. In some exemplary embodiments, the reaction conditions include the reaction temperature being approximately 80-100° C. In some exemplary embodiments, the reaction conditions include the reaction length being approximately 12-24 h. Exemplary reaction lengths include approximately 12, 13, 14, 15, 16, 17, 18, 19, 20, 21, 22, 23, or 24 h.

In the methods described herein, the reaction utilizes a Ligand (L), typically at loadings in various embodiments, including amounts from approximately 1-30 mol %. Exemplary Ligand amounts include approximately 5, 6, 7, 8, 9, 10, 11, 12, 13, 14, 15, 16, 17, 18, 19, 20, 21, 22, 23, 24, and 25 mol %. In some embodiments, the Ligand amount is 5 mol %. In some embodiments, the Ligand amount is 6 mol %. In some embodiments, the Ligand amount is 7 mol %. In some embodiments, the Ligand amount is 8 mol %. In some embodiments, the Ligand amount is 9 mol %. In some embodiments, the Ligand amount is 10 mol %. In some embodiments, the Ligand amount is 11 mol %. In some embodiments, the Ligand amount is 12 mol %. In some embodiments, the Ligand amount is 13 mol %. In some embodiments, the Ligand amount is 14 mol %. In some embodiments, the Ligand amount is 15 mol %. In some embodiments, the Ligand amount is 16 mol %. In some embodiments, the Ligand amount is 17 mol %. In some embodiments, the Ligand amount is 18 mol %. In some embodiments, the Ligand amount is 19 mol %. In some embodiments, the Ligand amount is 20 mol %. In some embodiments, the Ligand amount is 21 mol %. In some embodiments, the Ligand amount is 22 mol %. In some embodiments, the Ligand amount is 23 mol %. In some embodiments, the Ligand amount is 24 mol %. In some embodiments, the Ligand amount is 25 mol %.

In the methods described herein, the reaction utilizes a source of Pd(II), typically at catalytic loadings in various embodiments. Sources of palladium (II) can arise via reagents known in the art or commercially available. For example, one convenient source of palladium (II), per an embodiment, is Pd(OAc)₂. In some embodiments, the reaction conditions include the Pd source being Pd(OAc)₂. Pd(OAc)₂, PdCl₂C(CH₃CN)₂, Pd(TFA)₂, Pd(dba)₂, Pd(PhCN)₂Cl₂, or Pd(CH₃CN)₄(OTf)₂. In additional embodiments, the Pd source is Pd(CH₃CN)₄(BF₄)₂, PdCl₂, Pd(CH₃CN)₂Cl₂, [Pd(allyl)Cl]₂, or Pd(PPh₃)₂Cl₂. In some embodiments, the reaction conditions include the Pd source being Pd(OAc)₂. In some embodiments, the reaction conditions include the Pd source being PdCl₂C(CH₃CN)₂. In some embodiments, the reaction conditions include the Pd source being Pd(TFA)₂. In some embodiments, the reaction conditions include the Pd source being Pd(dba)₂. In some embodiments, the reaction conditions include the Pd source being Pd(PhCN)₂Cl₂. In some embodiments, the reaction conditions include the Pd source being Pd(CH₃CN)₄(OTf)₂. In additional embodiments, the Pd source is Pd(CH₃CN)₄(BF₄)₂, PdCl₂, Pd(CH₃CN)₂Cl₂, [Pd(allyl)Cl]₂, or Pd(PPh₃)₂Cl₂.

Palladium (II) loading can vary in accordance with factors known to those skilled in the art, such as overall reaction kinetics. Thus, in various embodiments, the source of palladium (II) is present in an amount of about 1 to about 15 mol %. In other embodiments, the amount is from about 7 to about 12 mol %. Exemplary amounts include 1, 2, 3, 4, 5, 6, 7, 8, 9, 10, 11, 12, 13, 14, and 15 mol %. In an embodiment, the amount is 10 mol %.

In some embodiments, the oxidant is at least one silver salt. Examples of suitable silver salts include but are not limited to the Ag salt is Ag₂CO₃, AgOAc, AgF, AgTFA, Ag₂O, or Ag₃PO₄. An illustrative silver salt, per one embodiment, is Ag₂CO₃. In some embodiments, the amount of the silver salt includes approximately 1.0, 1.5, 2.0, 2.5, or 3.0 equivalents, relative to the a-substituted carboxylic acid. An exemplary amount of the silver salt is approximately 2.0 equivalents relative to the a-substituted carboxylic acid.

In one embodiment, the oxidant is BQ. In one embodiment, the oxidant is K₂S₂O₈. In one embodiment, the oxidant is TBHP in H₂O. In one embodiment, the oxidant is TBHP in hexane. In one embodiment, the oxidant is ^{t}BuOO^{t}Bu. In one embodiment, the oxidant is ^{t}BuOOAc.

In the embodiments of the methods described herein, the reaction utilizes a base. Exemplary bases used include KF, Li₂CO₃, KOAc, LiOAc, NaOAc, KTFA, NaTFA, LiF, CsF, K₂HPO₄, and K₃PO₄. In some embodiments, the amount of the base is approximately 1.0, 1.5, 2.0, 2.5, or 3.0 equivalents relative to the a-substituted carboxylic acid. An exemplary amount of the base is approximately 2.0 equivalents relative to the a-substituted carboxylic acid.

In the embodiments of the methods described herein, the reaction utilizes a solvent comprising largely HFIP and another solvent. In some embodiments of the methods described herein, the reaction solvent is HFIP:MeCN at approximately 20:1. In some embodiments of the methods described herein, the reaction solvent is HFIP:MeCN at approximately 10:1. In some embodiments of the methods described herein, the reaction solvent is HFIP:MeCN at approximately 10:1.5. In some embodiments of the methods described herein, the reaction solvent is HFIP:DMSO at approximately 10:1. In some embodiments of the methods described herein, the reaction solvent is HFIP:CH₃(CH₂)₂CN at approximately 10:1.

In the methods described herein, the reactions are performed at elevated temperatures. Useful reaction temperatures include those between approximately 50 and 120° C., about 70 and 115° C., and approximately 80 and 110° C. Exemplary reaction temperatures include approximately 80, 90, 100, and 110° C.

Additional embodiments of the disclosure include the following non-limiting examples.

### EXAMPLES

Compounds of the invention can be made by a variety of methods depicted in the illustrative synthetic reactions described below in the Examples section.

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40. It should be appreciated that the synthetic reaction schemes shown in the Examples section are merely illustrative of some methods by which the compounds of the invention can be synthesized, and various modifications to these synthetic reaction schemes can be made and will be suggested to one skilled in the art having referred to the disclosure contained in this application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data. Unless specified to the contrary, the reactions described herein are typically conducted under an inert atmosphere at atmospheric pressure at a reaction temperature range of from about −78° C. to about 150° C., often from about 0° C. to about 125° C., and more often and conveniently at about room (or ambient) temperature, e.g., about 20° C.

Various substituents on the compounds of the invention can be present in the starting compounds, added to any one of the intermediates or added after formation of the final products by known methods of substitution or conversion reactions. If the substituents themselves are reactive, then the substituents can themselves be protected according to the techniques known in the art. A variety of protecting groups are known in the art, and can be employed. Examples of many of the possible groups can be found in “Protective Groups in Organic Synthesis” by Green et al., John Wiley and Sons, 1999. For example, nitro groups can be added by nitration and the nitro group can be converted to other groups, such as amino by reduction, and halogen by diazotization of the amino group and replacement of the diazo group with halogen. Acyl groups can be added by Friedel-Crafts acylation. The acyl groups can then be transformed to the corresponding alkyl groups by various methods, including the Wolff-Kishner reduction and Clemmenson reduction. Amino groups can be alkylated to form mono- and di-alkylamino groups; and mercapto and hydroxy groups can be alkylated to form corresponding ethers. Primary alcohols can be oxidized by oxidizing agents known in the art to form carboxylic acids or aldehydes, and secondary alcohols can be oxidized to form ketones. Thus, substitution or alteration reactions can be employed to provide a variety of substituents throughout the molecule of the starting material, intermediates, or the final product, including isolated products.

### 1. General Information:

Pd(OAc)₂ was purchased from Strem. Solvents were obtained from Sigma-Aldrich, Alfa-Aesar, and Acros, and used directly without further purification. Other reagents were purchased at the highest commercial quality and used without further purification, unless otherwise stated. Analytical thin layer chromatography was performed on 0.25 mm silica gel 60-F254 or Merck pre-coated aluminium-backed silica gel F254 plates. ¹H NMR spectra were recorded on Bruker AMX-400 or Bruker DRX-600 instruments. The following abbreviations (or combinations thereof) were used to explain multiplicities: s=singlet, d=doublet, t=triplet, q=quartet, m=multiplet, br=broad. Coupling constants, J, were reported in Hertz unit (Hz). ¹³C NMR spectra were recorded on Bruker DRX-600 and were fully decoupled by broad band proton decoupling. ¹⁹F NMR Spectra were recorded on Bruker AMX-400 spectrometer (376 MHz) and were fully decoupled by broad band proton decoupling. Chemical shifts were referenced to the appropriate residual solvent peaks (35). Column chromatography was performed using E. Merck silica (60, particle size 0.043-0.063 mm), and pTLC was performed on Merck silica plates (60F-254). High-resolution mass spectra (HRMS) were recorded on an Agilent Mass spectrometer using ESI-TOF (electrospray ionization-time of flight).

### EXPERIMENTALS

### 2. Experimental Section for β,γ-Dehydrogenation/Olefination Formation

### 2.1. Optimized Procedure for the Preparation of Ligands L8-L11

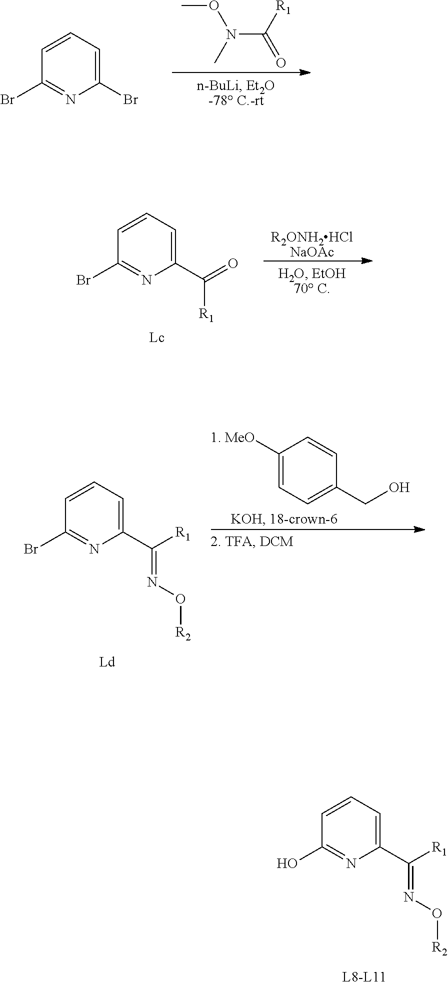

### Synthesis of Lc

A suspension of 2,6-dibromopyridine (2.35 g, 10 mmol, 1 equiv.) in Et₂O (50 mL) was cooled to −78° C. and n-BuLi (11 mmol, 1.1 equiv) was slowly added over a period of 5 min. The suspension became a clear yellow solution. After stirring for 30 min at this temperature, a solution of N-methoxy-N-methyamide (10 mmol, 1 equiv.) in Et₂O (10 mL) was added, and the reaction mixture was allowed to warm to room temperature. After stirring for 3.5 h, a saturated aqueous NH₄Cl solution was added. The layers were separated, and the aqueous layer was extracted once with Et₂O, dried over anhydrous Na₂SO₄, and concentrated under vacuum. The residue was purified by flash chromatography to give the pure product Lc (70% yield)

### Synthesis of Ld

To a solution of Lc (5 mmol, 1.0 equiv) in H₂O (45 mL) and EtOH (15 mL) at room temperature was added MeONH₂·HCl (1.13 g, 13.5 mmol, 2.7 equiv), NaOAc (1.8 g, 22 mmol, 4.4 equiv). The resulting solution was stirred for 2 h at 70° C. After cooling to room temperature, the mixture was extracted with EtOAc, dried over anhydrous Na₂SO₄, and concentrated under vacuum. The residue was purified by flash chromatography to give the pure product Ld (88% yield).

### Synthesis of L8-L11

A stirred mixture of Ld (4 mmol, 1.0 equiv), 4-methoxybenzyl alcohol (0.7 g, 4 mmol, 1.0 equiv), potassium hydroxide (0.22 g, 6 mmol, 1.2 equiv), 18-crown-6 (0.1 g, 0.4 mmol, 0.1 equiv) and toluene (20 mL) was heated under reflux for 12 hours. The cooled solution was evaporated under vacuum before 50 mL DCM was added. The organic layer was washed with brine twice and dried over anhydrous Na₂SO₄. The combined DCM solution was then transferred into a flask and 0.4 mL of trifluoroacetic acid was added. The mixture was stirred under room temperature for 1 hour before 60 mL of saturated NaHCO₃ was added. The mixture was extracted with CHCl₃ (60 mL×3) and the organic layers were combined and concentrated under vacuum. L8-L11 are purified by flash chromatography (EA: methanol=20:1) to afford a pale-yellow compound.

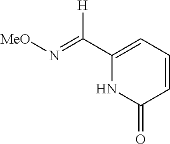 [SMILES: CO/N=C/c1cccc(=O)[nH]1]

### (E)-6-oxo-1,6-dihydropyridine-2-carbaldehyde O-methyl oxime (L8)

White solid, ¹H NMR (400 MHz, Chloroform-d) δ 7.79 (dd, J=7.3, 1.0 Hz, 1H), 7.71 (dd, J=8.2, 7.3 Hz, 1H), 6.91 (dd, J=8.2, 0.9 Hz, 1H), δ 5.91 (s, 1H), 3.95 (s, 3H); ¹³C NMR (151 MHz, CDCl₃) δ 166.8, 163.1, 147.0, 139.8, 116.0, 114.8, 53.6.

HRMS (ESI-TOF) m/z Calcd for C₇H₈N₂O₂⁺ [M+H]⁺ 153.0664. found 153.0661.

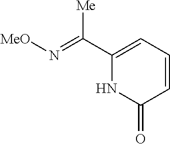 [SMILES: CO/N=C(\C)c1cccc(=O)[nH]1]

### (E)-6-(1-(methoxyimino)ethyl)pyridin-2 (1H)-one (L9)

White solid, ¹H NMR (400 MHz, Chloroform-d) δ 7.43-7.34 (m, 1H), 6.59 (d, J=9.2 Hz, 1H), 6.35 (d, J=3.4 Hz, 1H), 4.00 (s, 3H), 2.09 (s, 3H); ¹³C NMR (151 MHz, CDCl₃) δ 162.5, 146.4, 140.5, 140.0, 123.0, 105.1, 63.2, 10.2.

HRMS (ESI-TOF) m/z Calcd for C₈H₁₀N₂O₂⁺ [M+H]⁺ 167.0821. found 167.0817.

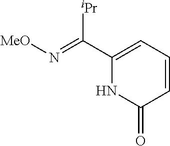 [SMILES: CO/N=C(/c1cccc(=O)[nH]1)C(C)C]

### (E)-6-(1-(methoxyimino)-2-methylpropyl)pyridin-2 (1H)-one (L10)

White solid, ¹H NMR (600 MHz, Chloroform-d) δ 7.45 (dd, J=9.3, 6.9 Hz, 1H), 6.60 (dd, J=9.3, 1.0 Hz, 1H), 6.27 (dd, J=7.0, 1.0 Hz, 1H), 3.95 (s, 3H), 2.89 (hept, J=6.8 Hz, 1H), 1.20 (d, J=6.8 Hz, 6H); ¹³C NMR (151 MHz, CDCl₃) δ 162.8, 153.2, 140.5, 138.7, 122.1, 106.2, 63.0, 32.6, 20.8.

HRMS (ESI-TOF) m/z Calcd for C₁₀H₁₄N₂O₂⁺ [M+H]⁺ 195.1134. found 195.1138.

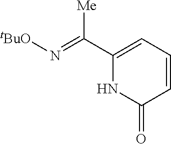 [SMILES: C/C(=N\OC(C)(C)C)c1cccc(=O)[nH]1]

### (E)-6-(1-(tert-butoxyimino)ethyl)pyridin-2 (1H)-one (L11)

White solid, ¹H NMR (600 MHz, Chloroform-d) δ 7.41 (dd, J=9.2, 6.9 Hz, 1H), 6.60 (dd, J=9.2, 0.9 Hz, 1H), 6.36 (dd, J=6.8, 1.0 Hz, 1H), 2.11 (s, 3H), 1.37 (s, 9H); ¹³C NMR (151 MHz, CDCl₃) δ 162.6, 145.4, 140.8, 140.6, 122.5, 104.8, 81.0, 27.7, 10.3.

HRMS (ESI-TOF) m/z Calcd for C₁₁H₁₆N₂O₂⁺ [M+H]⁺ 209.1290. found 209.1291.

### 2.2. Optimized Procedure for the Preparation of Ligands L12-L15

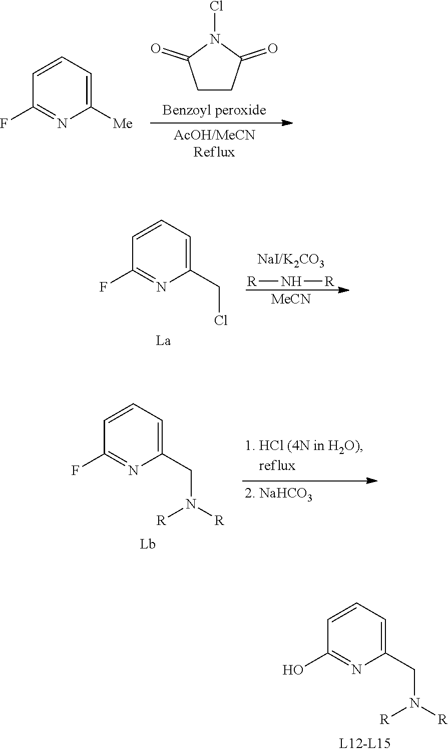

### Synthesis of La

To a solution of 2-fluoro-6-methylpyridine (50 mmol, 1.0 equiv.) in acetic acid (250 mL) and acetonitrile (125 mL) was added N-chlorosuccinimide (9.98 g, 75 mmol, 1.5 equiv.) and benzoyl peroxide (140 mg, 1 mmol, 0.02 equiv.). The resulting solution was stirred for 12 hours at 85° C. Upon completion, saturated aqueous NaHCO₃ was added and extracted with Et₂O three times after cooled to room temperature. The combined extracts were washed with brine, dried over anhydrous Na₂SO₄, and concentrated under vacuum. Flash chromatography gave La (85% yield).

### Synthesis of Lb

To a solution of La (5 mmol, 1.0 equiv) in acetonitrile (25 mL) was added NaI (75 mg, 0.5 mmol, 0.1 equiv.), K₂CO₃ (3.45 g, 25 mmol, 5 equiv.) and amine (10 mmol, 2 equiv.). The resulting solution was warmed to room temperature gradually after stirred for 12 h at 60° C. Upon completion, saturated aqueous NaCl solution was added and extracted with DCM three times. The combined extracts were dried over anhydrous Na₂SO₄, concentrated under vacuum. Flash chromatography gave Lb.

A suspension of Lb in 4N HCl in water (15 mL) was refluxed for 12 hours. Upon completion (determined by TLC monitoring), the reaction mixture was cooled to room temperature and quenched with saturated aqueous NaHCO₃ solution to neutral pH. The aqueous phase was extracted three times with dichloromethane. The combined organic layers were washed with brine, dried over anhydrous Na₂SO₄, and concentrated under vacuum. The residue was purified by flash chromatography (eluent: ethyl acetate/methanol=100/1 to 10/1) to give the pure product (L12-L15).

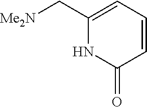 [SMILES: CN(C)Cc1cccc(=O)[nH]1]

### 6-((dimethylamino)methyl)pyridin-2 (1H)-one (L12)

White solid, ¹H NMR (600 MHz, Chloroform-d) δ 7.32 (dd, J=9.2, 6.7 Hz, 1H), 6.43 (dd, J=9.1, 1.0 Hz, 1H), 6.02 (dd, J=6.7, 1.1 Hz, 1H), 3.32 (s, 2H), 2.27 (s, 6H); ¹³C NMR (151 MHz, CDCl₃) δ 163.9, 145.4, 141.2, 119.4, 104.2, 60.0, 45.4.

HRMS (ESI-TOF) m/z Calcd for C₈H₁₂N₂O⁺ [M+H]⁺ 153.1028. found 153.1028.

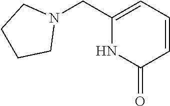 [SMILES: O=c1cccc(CN2CCCC2)[nH]1]

### 6-(pyrrolidin-1-ylmethyl)pyridin-2 (1H)-one (L13)

White solid, ¹H NMR (600 MHz, Chloroform-d) δ 7.32 (dd, J=9.1, 6.7 Hz, 1H), 6.41 (d, J=9.1 Hz, 1H), 6.02 (dd, J=6.7, 1.2 Hz, 1H), 3.53 (s, 2H), 2.55 (td, J=5.3, 4.0, 2.5 Hz, 4H), 1.84-1.76 (m, 4H); ¹³C NMR (151 MHz, CDCl₃) δ 163.8, 145.9, 141.2, 119.1, 103.6, 56.0, 54.1, 23.9.

HRMS (ESI-TOF) m/z Calcd for C₁₀H₁₄N₂O⁺ [M+H]⁺ 179.1184. found 179.1182.

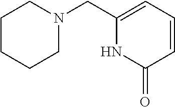 [SMILES: O=c1cccc(CN2CCCCC2)[nH]1]

### 6-(piperidin-1-ylmethyl)pyridin-2 (1/I)-one (L14)

White solid, ¹H NMR (600 MHz, Methanol-d₄) δ 7.54 (dd, J=9.1, 6.8 Hz, 1H), 6.44 (d, J=9.1 Hz, 1H), 6.34 (d, J=6.8 Hz, 1H), 3.42 (s, 2H), 2.46 (s, 4H), 1.68-1.57 (m, 4H), 1.50-1.46 (m, 2H); ¹³C NMR (151 MHz, MeOD) δ 166.1, 147.0, 143.5, 119.0, 108.2, 60.4, 55.4, 26.8, 25.0.

HRMS (ESI-TOF) m/z Calcd for C₁₁H₁₆N₂O⁺ [M+H]⁺ 193.1341. found 193.1340.

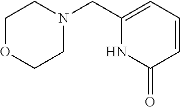 [SMILES: O=c1cccc(CN2CCOCC2)[nH]1]

### 6-(morpholinomethyl)pyridin-2 (1/I)-one (L15)

White solid, ¹H NMR (600 MHz, Chloroform-d) δ 7.32 (dd, J=9.2, 6.6 Hz, 1H), 6.45 (dd, J=9.3, 0.9 Hz, 1H), 6.03 (dt, J=6.7, 1.1 Hz, 1H), 3.77-3.67 (m, 4H), 3.39 (t, J=0.8 Hz, 2H), 2.49 (t, J=4.6 Hz, 4H); ¹³C NMR (151 MHz, CDCl₃) δ 164.1, 144.4, 141.2, 119.5, 104.9, 66.9, 59.2, 53.5.

HRMS (ESI-TOF) m/z Calcd for C₁₀H₁₄N₂O₂⁺ [M+H]⁺ 195.1134. found 195.1135.

### 2.3. Condition Screenings for β,γ-Dehydrogenation/Olefination Reaction

TABLE S1
Ligand Loading Effects^{s,b}

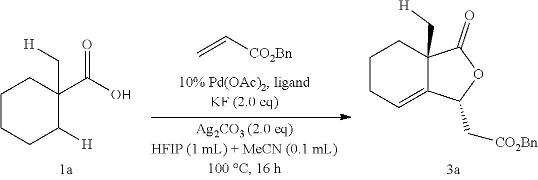 [SMILES: CC1(C(=O)O)CCCCC1 ; C=CC(=O)OCc1ccccc1 ; C[C@]12CCCC=C1[C@@H](CC(=O)OCc1ccccc1)OC2=O]

| entry | Ligand amount | yield (%) |
|---|---|---|
| 1 | 8 mol % | 30 |
| 2 | 10 mol % | 68 |
| 3 | 13 mol % | 80 |
| 4 | 15 mol % | 76 |
| 5 | 20 mol % | 76 |
| 6 | w/o | 0 |

ªConditions: 1a (0.1 mmol), benzyl acrylate 2a (0.2 mmol), Pd(OAc)₂ (10 mol %), L9, Ag₂CO₃ (2.0 equiv), KF (2.0 equiv), HFIP (1.0 mL), MeCN (0.1 mL), 100 °C, 16 h.
^{b}yields determined by ¹H NMR using dibromomethane as internal standard

TABLE S2
Pd Loading Effects^{s,b}

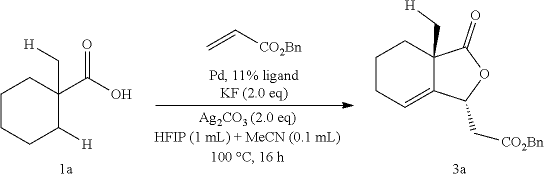 [SMILES: CC1(C(=O)O)CCCCC1 ; C=CC(=O)OCc1ccccc1 ; C[C@]12CCCC=C1[C@@H](CC(=O)OCc1ccccc1)OC2=O]

| entry | Pd | yield (%) |
|---|---|---|
| 1 | 10 mol % Pd(QAe)₂ | 72 |
| 2 | 7 mol % Pd(OAc)₂ | 68 |
| 3 | 5 mol % Pd(OAc)₂ | 60 |
| 4 | 15 mol % Pd(OAc)₂ | 72 |
| 5 | 20 mol % Pd(OAc)₂ | 72 |
| 6 | 10 mol % PdCl₂C(CH₃CN)₂ | 64 |
| 7 | 10 mol % Pd(TFA)₂ | 64 |
| 8 | 10 mol % Pd(dba)₂ | 64 |
| 9 | 10 mol % Pd(PhCN)₂Cl₂ | 72 |
| 10 | 10 mol % Pd(CH₃CN)₄(OTf)₂ | 54 |

ªConditions: 1a (0.1 mmol), benzyl acrylate 2a (0.2 mmol), Pd, L9 (1.1 equiv. relative to Pd), Ag₂CO₃ (2.0 equiv), KF (2.0 equiv), HFIP (1.0 mL), MeCN (0.1 mL), 100 °C, 16 h.
^{b}yields determined by ¹H NMR using dibromomethane as internal standard

TABLE S3
Solvent Effects^{a,b}

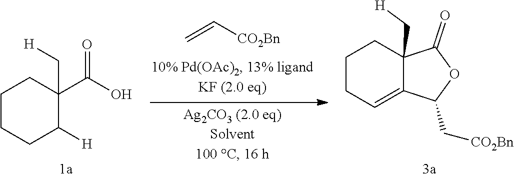 [SMILES: CC1(C(=O)O)CCCCC1 ; C=CC(=O)OCc1ccccc1 ; C[C@]12CCCC=C1[C@@H](CC(=O)OCc1ccccc1)OC2=O]

| Solvent | yield (%) |
|---|---|
| HFIP (1 mL) | 0 |
| HFIP (1 mL) + MeCN (0.05 mL) | 42 |
| HFIP (1 mL) + MeCN (0.1 mL) | 80 |
| HFIP (1 mL) + MeCN (0.15 mL) | 68 |
| HFIP (1 mL) + DMSO (0.1 mL) | 12 |
| HFIP (1 mL) + CH3(CH₂)₂CN (0.1 mL) | 64 |
| HFIP (1 mL) + THF | 0 |
| HFIP (1 mL) + DMF (0.1 mL) | 0 |
| HFIP (1 mL) + Dioxane (0.1 mL) | 0 |
| HFIP (1 mL) + DMA (0.1 mL) | 0 |
| HFIP (1 mL) + DCE (0.1 mL) | 0 |
| HFIP (1 mL) + Tolene (0.1 mL) | 0 |
| HFIP (1 mL) + MeOH (0.1 mL) | 0 |
| HFIP (1 mL) + EA (0.1 mL) | 0 |

ªConditions: 1a (0.1 mmol), benzyl acrylate 2a (0.2 mmol), Pd(OAc)₂ (10 mol %), L9 (1.3 equiv. relative to Pd), Ag₂CO₃ (2.0 equiv), KF (2.0 equiv), solvent, 100 °C, 16 h.
^{b}yields determined by ¹H NMR using dibromomethane as internal standard

TABLE S4
Base Effects^{a,b}

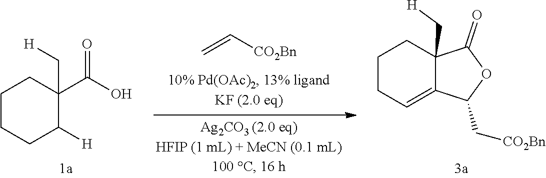 [SMILES: CC1(C(=O)O)CCCCC1 ; C=CC(=O)OCc1ccccc1 ; C[C@]12CCCC=C1[C@@H](CC(=O)OCc1ccccc1)OC2=O]

| Base | yield (%) |
|---|---|
| KF (1.0 eq) | 62 |
| KF (1.5 eq) | 66 |
| KF (2.0 eq) | 80 |
| KF (2.5 eq) | 76 |
| KF (3.0 eq) | 68 |
| Li₂CO₃ | 64 |
| KOAC | 62 |
| LiOAc | 42 |

ªConditions: 1a (0.1 mmol), benzyl acrylate 2a (0.2 mmol), Pd(OAc)₂ (10 mol %), L9 (1.3 equiv. relative to Pd), Ag₂CO₃ (2.0 equiv), KF (2.0 equiv), solvent, 100 °C, 16 h.
^{b}yields determined by ¹H NMR using dibromomethane as internal standard

TABLE S5
Screenings of Oxidants^{a,b}

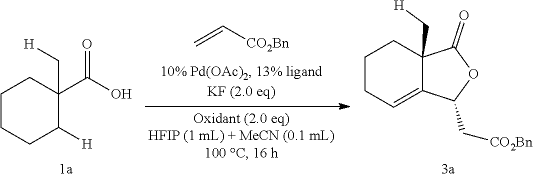 [SMILES: CC1(C(=O)O)CCCCC1 ; C=CC(=O)OCc1ccccc1 ; C[C@]12CCCC=C1[C@@H](CC(=O)OCc1ccccc1)OC2=O]

| Oxidants | yield (%) |
|---|---|
| AgCO₃ (1.0 eq) | 48 |
| AgCO₃ (1.5 eq) | 60 |
| AgCO₃ (2.0 eq) | 80 |
| AgCO₃ (3.0 eq) | 44 |
| AgOAc | 28 |
| AgF | 26 |
| AgTFA | 8 |
| Ag₂O | 72 |
| Ag₃PO₄ | 48 |

ªConditions: 1a (0.1 mmol), benzyl acrylate 2a (0.2 mmol), Pd(OAc)₂ (10 mol %), L9 (1.3 equiv. relative to Pd), Ag₂CO₃ (2.0 equiv), KF (2.0 equiv), solvent, 100 °C, 16 h.
^{b}yields determined by ¹H NMR using dibromomethane as internal standard

### 2.4. General Procedure for β-C(sp³)-H Dehydrogenation:

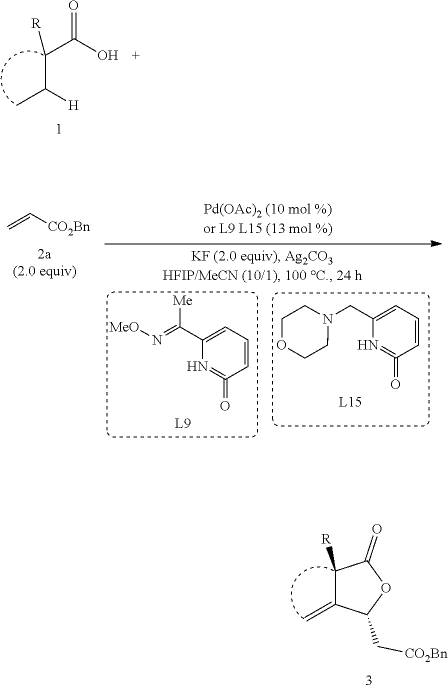

In a sealed tube equipped with a magnetic stir bar was charged with Pd(OAc)₂ (2.2 mg, 10 mol %), L9 (2.2 mg, 13 mol %) or L15 (2.8 mg, 13 mol %), the appropriate carboxylic acid substrate (0.10 mmol), benzyl acrylate (32.4 mg, 0.2 mmol), Ag₂CO₃ (55.0 mg, 0.2 mmol), and KF (14.8 mg, 0.2 mmol). HFIP (1 mL) and MeCN (0.1 mL) were then added. The reaction mixture was then stirred at the rate of 600 rpm at 100° C. for 24 h. After being allowed to cool to room temperature, the mixture was diluted with ethyl acetate and acidified with 0.5 mL of formic acid. The mixture was passed through a pad of Celite with acetone as the eluent to remove any insoluble precipitate. The resulting solution was concentrated, and the residual mixture was isolated using pTLC (hexane:ethyl acetate=8:1 to 5:1).

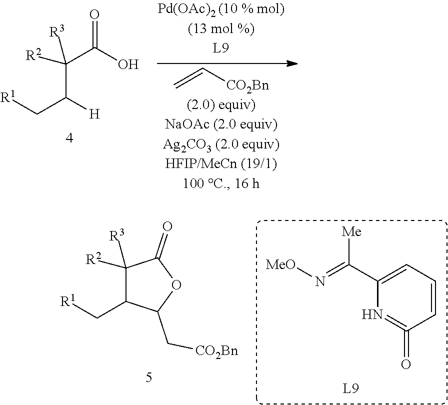

In a sealed tube equipped with a magnetic stir bar was charged with Pd(OAc)₂ (2.2 mg, 10 mol %), L9 (2.2 mg, 13 mol %), the appropriate carboxylic acid substrate (0.10 mmol), benzyl acrylate (32.4 mg, 0.2 mmol), Ag₂CO₃ (55.0 mg, 0.2 mmol), and NaOAc (16.5 mg, 0.2 mmol). HFIP (0.95 mL) and MeCN (0.05 mL) were then added. The reaction mixture was then stirred at the rate of 600 rpm at 100° C. for 16 h. After being allowed to cool to room temperature, the mixture was diluted with ethyl acetate and acidified with 0.5 mL of formic acid. The mixture was passed through a pad of Celite with acetone as the eluent to remove any insoluble precipitate. The resulting solution was concentrated, and the residual mixture was isolated using pTLC (hexane:ethyl acetate=8:1 to 5:1).

### 2.5. Substrate Scope for β,γ-Dehydrogenation/Olefination Reaction

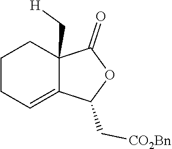 [SMILES: C[C@]12CCCC=C1[C@@H](CC(=O)OCc1ccccc1)OC2=O]

### Benzyl 2-((3aS)-3a-methyl-3-oxo-1,3,3a,4,5,6-hexahydroisobenzofuran-1-yl)acetate (3a)

Following General Procedure on 0.1 mmol scale. The product was obtained as colorless oil (80% yield). ¹H NMR (600 MHz, Chloroform-d) δ 7.41-7.31 (m, 5H), 5.56 (q, J=3.1 Hz, 1H), 5.44 (td, J=5.0, 2.4 Hz, 1H), 5.22-5.14 (m, 2H), 2.88 (dd, J=16.4, 4.6 Hz, 1H), 2.75 (dd, J=16.5, 7.7 Hz, 1H), 2.16 (dp, J=19.1, 3.5 Hz, 1H), 2.01 (dtt, J=18.6, 7.2, 3.2 Hz, 1H), 1.89 (dt, J=13.0, 3.4 Hz, 1H), 1.78 (ddt, J=14.1, 6.9, 3.5 Hz, 1H), 1.68 (tddd, J=14.2, 10.5, 6.6, 3.5 Hz, 1H), 1.42 (td, J=13.5, 3.7 Hz, 1H), 1.36 (s, 3H); ¹³C NMR (151 MHz, CDCl₃) δ 179.6, 169.9, 139.3, 135.5, 128.8, 128.6, 120.7, 76.5, 67.1, 41.5, 37.9, 28.7, 24.1, 22.4, 17.2; HRMS (ESI-TOF) m/z Calcd for C₁₈H₂₀O₄⁺ [M+H]⁺ 301.1440. found 301.1438.

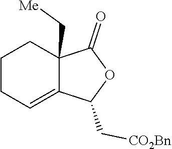 [SMILES: CC[C@]12CCCC=C1[C@@H](CC(=O)OCc1ccccc1)OC2=O]

### Benzyl 2-((3aS)-3a-ethyl-3-oxo-1,3,3a,4,5,6-hexahydroisobenzofuran-1-yl)acetate (3b)

Following General Procedure on 0.1 mmol scale. The product was obtained as colorless oil (78% yield). ¹H NMR (600 MHz, Chloroform-d) δ 7.41-7.30 (m, 5H), 5.58 (q, J=3.2 Hz, 1H), 5.42 (ddq, J=7.4, 5.1, 2.6 Hz, 1H), 5.22-5.12 (m, 2H), 2.88 (dd, J=16.4, 4.5 Hz, 1H), 2.73 (dd, J=16.4, 7.6 Hz, 1H), 2.20-2.10 (m, 1H), 2.08-1.96 (m, 2H), 1.80 (dq, J=14.9, 7.5 Hz, 1H), 1.75-1.60 (m, 3H), 1.30 (td, J=13.4, 4.4 Hz, 1H), 1.00 (t, J=7.5 Hz, 3H); ¹³C NMR (151 MHz, CDCl₃) δ 178.5, 169.9, 139.7, 135.5, 128.7, 128.5, 121.0, 76.8, 67.0, 45.4, 38.4, 28.7, 25.8, 23.8, 16.8, 9.0; HRMS (ESI-TOF) m/z Calcd for C₁₉H₂₃O₄⁺ [M+H]⁺ 315.1596. found 315.1597.

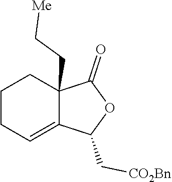 [SMILES: CCC[C@]12CCCC=C1[C@@H](CC(=O)OCc1ccccc1)OC2=O]

### Benzyl 2-((3aS)-3-oxo-3a-propyl-1,3,3a,4,5,6-hexahydroisobenzofuran-1-yl)acetate (3c)

Following General Procedure on 0.1 mmol scale. The product was obtained as colorless oil (80% yield). ¹H NMR (600 MHz, Chloroform-d) δ 7.41-7.30 (m, 5H), 5.67 (t, J=3.7 Hz) and 5.57 (td, J=3.6, 1.9 Hz, 1H), 5.42 (ddq, J=7.6, 5.1, 2.6 Hz, 1H) and 5.30 (td, J=7.4, 1.7 Hz), 5.21-5.13 (m, 2H), 2.99 (dd, J=15.9, 7.6 Hz) and 2.87 (dd, J=16.4, 4.6 Hz, 1H), 2.73 (dd, J=16.4, 7.5 Hz, 1H), 2.27-1.96 (m, 3H), 1.78-1.55 (m, 4H), 1.50-1.15 (m, 3H), 0.89 (dt, J=15.4, 7.3 Hz, 3H); ¹³C NMR (151 MHz, CDCl₃) δ 178.8, 169.9, 139.7, 135.6, 128.8, 128.6, 128.6, 120.9, 76.9, 67.0, 45.3, 38.4, 38.2, 26.6, 23.8, 17.9, 16.9, 14.5; HRMS (ESI-TOF) m/z Calcd for C₂₀H₂₄O₄⁺ [M+H]⁺ 329.1753. found 329.1759.

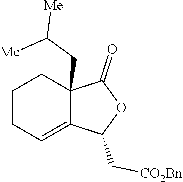 [SMILES: CC(C)C[C@]12CCCC=C1[C@@H](CC(=O)OCc1ccccc1)OC2=O]

### Benzyl 2-((3aR)-3a-isobutyl-3-oxo-1,3,3a,4,5,6-hexahydroisobenzofuran-1-yl)acetate (3d)

Following General Procedure on 0.1 mmol scale. The product was obtained as colorless oil (66% yield). ¹H NMR (600 MHz, Chloroform-d) δ 7.43-7.30 (m, 5H), 5.56 (hept, J=1.6 Hz, 1H), 5.43 (dddd, J=6.8, 5.2, 3.6, 2.0 Hz, 1H), 5.17 (qd, J=12.2, 1.4 Hz, 2H), 2.87 (ddd, J=16.5, 4.7, 1.3 Hz, 1H), 2.73 (ddd, J=16.4, 7.5, 1.5 Hz, 1H), 2.20-2.10 (m, 1H), 2.08-1.97 (m, 2H), 1.84 (dp, J=13.6, 6.8 Hz, 1H), 1.76-1.66 (m, 2H), 1.61 (dt, J=5.7, 1.9 Hz, 2H), 1.37-1.29 (m, 1H), 0.94 (ddd, J=23.3, 6.7, 1.4 Hz, 6H); ¹³C NMR (151 MHz, CDCl₃) δ 179.0, 169.9, 139.9, 135.5, 128.7, 128.6, 128.5, 120.9, 76.7, 67.0, 44.9, 44.5, 38.3, 27.2, 24.8, 24.6, 24.5, 23.7, 16.9; HRMS (ESI-TOF) m/z Calcd for C₂₁H₂₇O₄⁺ [M+H]⁺ 329.1753. found 329.1749.

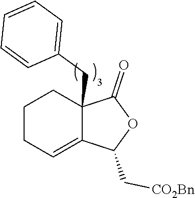

### Benzyl 2-((3aR)-3-oxo-3a-(3-phenylpropyl)-1,3,3a,4,5,6-hexahydroisobenzofuran-1-yl)acetate (3e)

Following General Procedure on 0.1 mmol scale. The product was obtained as colorless oil (72% yield). ¹H NMR (600 MHz, Chloroform-d) δ 7.40-7.30 (m, 5H), 7.28-7.25 (m, 2H), 7.16 (dd, J=21.4, 7.5 Hz, 3H), 5.56 (q, J=2.9 Hz, 1H), 5.39 (tt, J=5.0, 2.4 Hz, 1H), 5.21-5.11 (m, 2H), 2.85 (dd, J=16.5, 4.6 Hz, 1H), 2.71 (dd, J=16.5, 7.6 Hz, 1H), 2.67-2.50 (m, 2H), 2.17-2.08 (m, 1H), 2.06-1.94 (m, 2H), 1.79-1.54 (m, 6H), 1.30 (td, J=13.7, 4.0 Hz, 1H); ¹³C NMR (151 MHz, CDCl₃) δ 178.6, 169.9, 141.6, 139.5, 135.5, 128.7, 128.6, 128.5, 128.5, 126.0, 121.1, 76.8, 67.0, 45.0, 38.4, 36.1, 35.3, 26.4, 26.0, 23.7, 16.8; HRMS (ESI-TOF) m/z Calcd for C₂₆H₂₈O₄⁺ [M+H]⁺ 405.2066. found 405.2065.

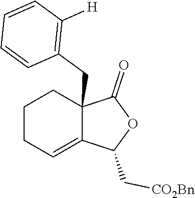 [SMILES: O=C(C[C@H]1OC(=O)[C@@]2(Cc3ccccc3)CCCC=C12)OCc1ccccc1]

### Benzyl 2-((3aR)-3a-benzyl-3-oxo-1,3,3a,4,5,6-hexahydroisobenzofuran-1-yl)acetate (3f)

Following General Procedure on 0.1 mmol scale. The product was obtained as colorless oil (75% yield). ¹H NMR (600 MHz, Chloroform-d) δ 7.39-7.32 (m, 3H), 7.29 (d, J=7.7 Hz, 2H), 7.27-7.22 (m, 3H), 7.16 (dt, J=6.8, 1.7 Hz, 2H), 5.63 (tt, J=3.5, 1.6 Hz, 1H), 5.13-5.03 (m, 2H), 4.25 (tq, J=5.0, 2.5 Hz, 1H), 3.04-2.93 (m, 2H), 2.70 (ddd, J=16.5, 5.0, 1.4 Hz, 1H), 2.57 (ddd, J=16.5, 7.1, 1.5 Hz, 1H), 2.31-2.20 (m, 1H), 2.14-1.99 (m, 2H), 1.90-1.75 (m, 2H), 1.45-1.35 (m, 1H); ¹³C NMR (151 MHz, CDCl₃) δ 178.8, 169.5, 138.6, 136.0, 135.5, 129.9, 128.7, 128.6, 128.5, 128.5, 127.4, 122.2, 77.3, 66.9, 47.8, 43.1, 38.3, 28.2, 23.7, 16.9; HRMS (ESI-TOF) m/z Calcd for C₂₄H₂₄O₄⁺ [M+H]⁺ 377.1753. found 377.1747.

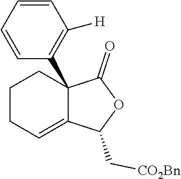 [SMILES: O=C(C[C@H]1OC(=O)[C@@]2(c3ccccc3)CCCC=C12)OCc1ccccc1]

### Benzyl 2-((3aR)-3-oxo-3a-phenyl-1,3,3a,4,5,6-hexahydroisobenzofuran-1-yl)acetate (3g)

Following General Procedure on 0.1 mmol scale. The product was obtained as colorless oil (62% yield). ¹H NMR (600 MHz, Chloroform-d) δ 7.49-7.44 (m, 2H), 7.40-7.27 (m, 8H), 5.95 (p, J=2.3 Hz, 1H), 5.31 (ddq, J=7.6, 4.9, 2.5 Hz, 1H), 5.21-5.14 (m, 2H), 2.97 (ddd, J=16.5, 4.8, 1.4 Hz, 1H), 2.83 (ddd, J=16.5, 7.6, 1.6 Hz, 1H), 2.27-2.15 (m, 2H), 2.09 (dddd, J=18.9, 10.5, 6.6, 2.9 Hz, 1H), 1.75-1.67 (m, 1H), 1.61-1.56 (m, 1H), 1.33-1.26 (m, 1H); ¹³C NMR (151 MHz, CDCl₃) δ 177.0, 169.8, 138.1, 137.0, 135.5, 128.83, 128.76, 128.6, 128.0, 127.6, 123.9, 76.7, 67.1, 50.2, 37.6, 31.9, 24.1, 16.5; HRMS (ESI-TOF) m/z Calcd for C₂₂H₂₃O₄⁺ [M+H]⁺ 363.1596. found 363.1595.

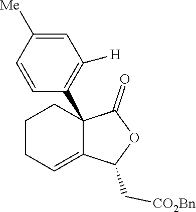 [SMILES: Cc1ccc([C@]23CCCC=C2[C@@H](CC(=O)OCc2ccccc2)OC3=O)cc1]

### Benzyl 2-((3aR)-3-oxo-3a-(p-tolyl)-1,3,3a,4,5,6-hexahydroisobenzofuran-1-yl)acetate (3h)

Following General Procedure on 0.1 mmol scale. The product was obtained as colorless oil (58% yield). ¹H NMR (600 MHz, Chloroform-d) δ 7.40-7.31 (m, 7H), 7.16 (d, J=7.7 Hz, 2H), 5.93 (hept, J=1.6 Hz, 1H), 5.31 (dq, J=7.8, 2.6 Hz, 1H), 5.17 (t, J=2.1 Hz, 2H), 2.96 (ddd, J=16.5, 4.9, 1.4 Hz, 1H), 2.82 (ddd, J=16.5, 7.6, 1.6 Hz, 1H), 2.34 (s, 3H), 2.25-2.15 (m, 2H), 2.08 (dtt, J=18.7, 7.7, 3.4 Hz, 1H), 1.72-1.65 (m, 1H), 1.58 (ddd, J=14.6, 7.3, 3.7 Hz, 1H), 1.34-1.27 (m, 1H); ¹³C NMR (151 MHz, CDCl₃) δ 177.1, 169.8, 137.8, 137.1, 135.5, 135.1, 129.5, 128.7, 128.6, 127.5, 123.6, 76.7, 67.0, 49.9, 37.6, 31.9, 24.1, 21.2, 16.5; HRMS (ESI-TOF) m/z Calcd for C₂₄H₂₅O₄⁺ [M+H]⁺ 377.1753. found 377.1747.

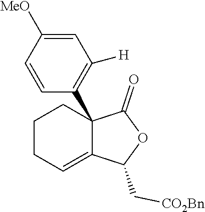 [SMILES: COc1ccc([C@]23CCCC=C2[C@@H](CC(=O)OCc2ccccc2)OC3=O)cc1]

### Benzyl 2-((3aR)-3a-(4-methoxyphenyl)-3-oxo-1,3,3a,4,5,6-hexahydroisobenzofuran-1-yl)acetate (3i)

Following General Procedure on 0.1 mmol scale. The product was obtained as colorless oil (42% yield). ¹H NMR (600 MHz, Chloroform-d) δ 7.42-7.30 (m, 7H), 6.91-6.85 (m, 2H), 5.92 (h, J=1.8 Hz, 1H), 5.30 (qt, J=4.7, 2.4 Hz, 1H), 5.17 (t, J=1.7 Hz, 2H), 3.80 (d, J=1.7 Hz, 3H), 2.96 (ddd, J=16.4, 4.9, 1.5 Hz, 1H), 2.82 (ddd, J=16.5, 7.5, 1.7 Hz, 1H), 2.18 (dt, J=12.6, 3.4 Hz, 2H), 2.14-2.02 (m, 1H), 1.72-1.64 (m, 1H), 1.58 (m, 1H), 1.36-1.27 (m, 1H); ¹³C NMR (151 MHz, CDCl₃) δ 177.2, 169.8, 159.4, 137.3, 135.5, 130.0, 128.8, 128.7, 128.6, 123.6, 114.2, 76.7, 67.1, 55.5, 49.5, 37.6, 31.9, 24.1, 16.5; HRMS (ESI-TOF) m/z Calcd for C₂₄H₂₅O₅⁺ [M+H]⁺ 393.1702. found 393.1705.

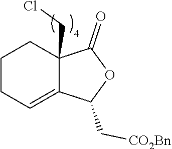

### Benzyl 2-((3aR)-3a-(4-chlorobutyl)-3-oxo-1,3,3a,4,5,6-hexahydroisobenzofuran-1-yl)acetate (3j)

Following General Procedure on 0.1 mmol scale. The product was obtained as colorless oil (70% yield). ¹H NMR (600 MHz, Chloroform-d) δ 7.42-7.29 (m, 5H), 5.59 (td, J=3.6, 2.0 Hz, 1H), 5.42 (ddt, J=9.9, 4.8, 2.4 Hz, 1H), 5.23-5.12 (m, 2H), 3.52 (t, J=6.6 Hz, 2H), 2.88 (dd, J=16.5, 4.6 Hz, 1H), 2.73 (dd, J=16.5, 7.5 Hz, 1H), 2.21-2.12 (m, 1H), 2.08-1.98 (m, 2H), 1.81-1.62 (m, 6H), 1.62-1.49 (m, 2H), 1.32 (td, J=13.5, 4.1 Hz, 1H); ¹³C NMR (151 MHz, CDCl₃) δ 177.4, 168.8, 138.4, 134.5, 127.8, 127.58, 127.57, 120.3, 75.9, 66.1, 44.0, 43.6, 37.4, 34.0, 31.8, 25.3, 22.8, 20.9, 15.8; HRMS (ESI-TOF) m/z Calcd for C₂₁H₂₆CO₄⁺ [M+H]⁺+377.1520. found 377.1526.

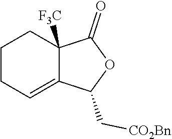 [SMILES: O=C(C[C@H]1OC(=O)[C@@]2(C(F)(F)F)CCCC=C12)OCc1ccccc1]

### Benzyl 2-((3aR)-3-oxo-3a-(trifluoromethyl)-1,3,3a,4,5,6-hexahydroisobenzofuran-1-yl)acetate (3k)

Following General Procedure on 0.1 mmol scale. The product was obtained as colorless oil (46% yield). ¹H NMR (600 MHz, Chloroform-d) δ 7.41-7.32 (m, 5H), 6.15 (ddd, J=4.7, 3.0, 1.2 Hz) and 5.98 (ddd, J=5.0, 3.4, 2.0 Hz, 1H), 5.55-5.45 (m, 1H), 5.21-5.13 (m, 2H), 3.01 (dd, J=16.6, 6.9 Hz) and 2.96 (dd, J=16.5, 4.8 Hz, 1H), 2.81 (dd, J=16.5, 7.0 Hz, 1H), 2.51 (ddd, J=13.8, 4.7, 2.8 Hz) and 2.44 (ddd, J=14.1, 4.6, 3.2 Hz, 1H), 2.27 (ddtd, J=19.4, 7.2, 3.4, 1.8 Hz, 1H), 2.10 (ddddd, J=21.8, 12.1, 7.4, 4.4, 2.5 Hz, 1H) and 2.01 (q, J=6.4 Hz), 1.92-1.78 (m, 1H), 1.73 (ddtt, J=12.7, 7.9, 3.2, 1.6 Hz, 1H) and 1.63 (q, J=7.3 Hz), 1.43 (tdq, J=14.1, 4.5, 2.1 Hz, 1H); ¹³C NMR (151 MHz, CDCl₃) δ 170.7 (d, J=2.4 Hz), 169.1, 135.4, 131.9, 130.6, 128.8, 128.7, 128.5, 124.8 (q, J=285.6 Hz), 78.1, 67.2, 50.1 (q, J=26.7 Hz), 38.0, 24.0, 22.9, 16.30 (d, J=2.2 Hz); HRMS (ESI-TOF) m/z Calcd for C₁₈H₁₇F₃O₄⁺ [M+H]⁺ 355.1157. found 355.1147.

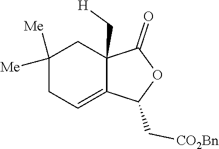 [SMILES: CC1(C)CC=C2[C@@H](CC(=O)OCc3ccccc3)OC(=O)[C@@]2(C)C1]

### Benzyl 2-((3aS)-3a,5,5-trimethyl-3-oxo-1,3,3a,4,5,6-hexahydroisobenzofuran-1-yl)acetate (31)

Following General Procedure on 0.1 mmol scale. The product was obtained as colorless oil (66% yield). ¹H NMR (600 MHz, Chloroform-d) δ 7.42-7.30 (m, 5H), 5.84 (ddd, J=6.0, 3.7, 1.2 Hz) and 5.68 (ddd, J=5.6, 3.3, 1.9 Hz, 1H), 5.50-5.43 (m, 1H) and 5.38-5.33 (m, OH), 5.22-5.15 (m, 2H), 3.00 (dd, J=15.9, 7.6 Hz) and 2.90 (dd, J=16.4, 4.8 Hz, 1H), 2.78 (dd, J=16.4, 7.7 Hz, 1H), 1.92 (dt, J=17.0, 3.3 Hz, 1H), 1.87 (dd, J=14.1, 1.5 Hz, 1H), 1.82 (ddt, J=17.0, 5.9, 1.4 Hz, 1H), 1.51 (d, J=14.1 Hz, 1H), 1.39 (s, 3H), 1.09 (s, OH) and 1.04 (s, 3H), 0.87 (s, OH) and 0.81 (s, 3H); ¹³C NMR (151 MHz, CDCl₃) δ 177.8, 168.9, 138.0, 134.5, 127.8, 127.60, 127.55, 120.3, 74.9, 66.1, 42.3, 41.5, 37.0, 36.9, 29.6, 29.5, 27.9, 23.6; HRMS (ESI-TOF) m/z Calcd for C₂₀H₂₄O₄⁺ [M+H]⁺ 329.1753. found 329.1753.

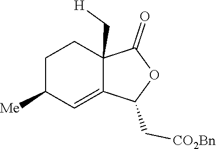 [SMILES: C[C@@H]1C=C2[C@@H](CC(=O)OCc3ccccc3)OC(=O)[C@@]2(C)CC1]

### Benzyl 2-((3aS,6R)-3a,6-dimethyl-3-oxo-1,3,3a,4,5,6-hexahydroisobenzofuran-1-yl)acetate (3m)

Following General Procedure on 0.1 mmol scale. The product was obtained as colorless oil (60% yield). ¹H NMR (600 MHz, Chloroform-d) δ 7.40-7.31 (m, 5H), 5.46-5.41 (m, 2H), 5.22-5.13 (m, 2H), 2.88 (dd, J=16.5, 4.3 Hz, 1H), 2.74 (ddd, J=16.1, 7.7, 5.7 Hz, 1H), 2.34 (ddtd, J=9.6, 7.3, 3.6, 2.0 Hz) and 2.24-2.14 (m, 1H), 1.89 (dddt, J=17.4, 11.0, 7.2, 3.4 Hz) and 1.73 (dt, J=13.1, 3.6 Hz, 2H), 1.55-1.44 (m) and 1.35 (d, J=3.7 Hz, 5H), 1.02 (d, J=7.0 Hz) and 0.95 (d, J=7.3 Hz, 3H); ¹³C NMR (151 MHz, CDCl₃) δ 179.4, 169.8, 138.9 and 138.7, 135.6 and 135.5, 128.8, 128.6, 128.58 and 128.56, 126.7 and 126.5, 76.4 and 76.3, 67.1 and 67.0, 41.9 and 41.8, 38.0 and 37.8, 31.0 and 29.6, 27.9 and 27.0, 24.9 and 24.5, 22.8 and 22.1, 21.7 and 21.3; HRMS (ESI-TOF) m/z Calcd for C₁₉H₂₃O₄⁺ [M+H]⁺ 315.1591. found 315.1581.

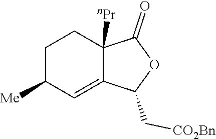

### Benzyl 2-((3aS,6R)-6-methyl-3-oxo-3a-propyl-1,3,3a,4,5,6-hexahydroisobenzofuran-1-yl)acetate (3n)

Following General Procedure on 0.1 mmol scale. The product was obtained as colorless oil (68% yield). ¹H NMR (600 MHz, Chloroform-d) δ 7.40-7.30 (m, 5H), 5.52 (d, J=2.9 Hz) and 5.46-5.39 (m, 2H), 5.21-5.13 (m, 2H), 2.99 (ddd, J=16.0, 7.7, 2.1) and 2.90-2.84 (m, 1H), 2.75-2.68 (m, 1H), 2.36-2.16 (m, 1H), 2.05-1.80 (m, 2H), 1.72-1.60 (m, 2H), 1.49-1.33 (m, 4H), 1.04-0.85 (m, 6H); ¹³C NMR (151 MHz, CDCl₃) δ 178.6, 169.9, 139.1, 135.5, 128.8, 128.6, 128.5, 127.0, 76.6, 67.0, 45.7, 38.5, 38.1, 27.8, 24.7, 23.2, 21.6, 17.9, 14.4; HRMS (ESI-TOF) m/z Calcd for C₂₁H₂₇O₄⁺ [M+H]⁺ 315.1596. found 315.1604.

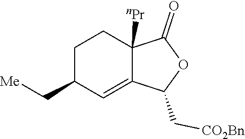

### Benzyl 2-((3aS,6R)-6-ethyl-3-oxo-3a-propyl-1,3,3a,4,5,6-hexahydroisobenzofuran-1-yl)acetate (3o)

Following General Procedure on 0.1 mmol scale. The product was obtained as colorless oil (66% yield). ¹H NMR (600 MHz, Chloroform-d) δ 7.43-7.32 (m, 5H), 5.55-5.50 (m, 1H), 5.45 (dddd, J=7.6, 4.5, 3.2, 2.0 Hz, 1H), 5.23-5.16 (m, 2H), 2.90 (dd, J=16.5, 4.5 Hz, 1H), 2.74 (dd, J=16.5, 7.6 Hz, 1H), 2.11-2.04 (m, 1H), 1.82-1.91 (m, 2H), 1.74-1.61 (m, 2H), 1.52-1.38 (m, 4H), 1.37-1.28 (m, 2H), 0.93 (td, J=7.3, 3.5 Hz, 6H); ¹³C NMR (151 MHz, CDCl₃) δ 177.7, 168.9, 138.7, 134.6, 127.8, 127.6, 127.6, 124.7, 75.7, 66.0, 44.9, 37.6, 37.1, 33.4, 27.7, 23.1, 21.4, 16.9, 13.4, 11.0; HRMS (ESI-TOF) m/z Calcd for C₂₂H₂₈O₄⁺ [M+H]⁺ 357.2066. found 357.2062.

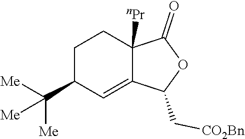

### Benzyl 2-((3aS,6R)-6-(tert-butyl)-3-oxo-3a-propyl-1,3,3a,4,5,6-hexahydroisobenzofuran-1-yl)acetate (3p)

Following General Procedure on 0.1 mmol scale. The product was obtained as colorless oil (70% yield). ¹H NMR (600 MHz, Chloroform-d) δ 7.40-7.31 (m, 5H), 5.75 (t, J=2.4 Hz, 1H), 5.46-5.40 (m, 1H), 5.22-5.13 (m, 2H), 2.90 (dd, J=16.4, 4.4 Hz, 1H), 2.73 (dd, J=16.5, 7.6 Hz, 1H), 1.85 (tdd, J=7.4, 5.1, 2.3 Hz, 2H), 1.80-1.71 (m, 1H), 1.66 (ddd, J=13.6, 10.3, 6.9 Hz, 1H), 1.57-1.47 (m, 3H), 1.44-1.35 (m, 2H), 0.89 (d, J=17.7 Hz, 12H); 13C NMR (151 MHz, CDCl₃) δ 179.0, 169.8, 141.3, 135.5, 128.78, 128.75, 128.6, 123.6, 76.9, 67.1, 45.8, 42.5, 38.8, 38.3, 34.0, 27.7, 26.8, 19.6, 18.0, 14.4; HRMS (ESI-TOF) m/z Calcd for C₂₄H₃₃O₄⁺ [M+H]⁺ 385.2379. found 385.2375.

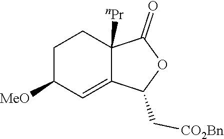

### Benzyl 2-((3aS,6R)-6-methoxy-3-oxo-3a-propyl-1,3,3a,4,5,6-hexahydroisobenzofuran-1-yl)acetate (3q)

Following General Procedure on 0.1 mmol scale. The product was obtained as colorless oil (62% yield). ¹H NMR (600 MHz, Chloroform-d) δ 7.41-7.29 (m, 5H), 5.77 (dt, J=2.9, 1.2 Hz) and 5.67 (ddd, J=3.2, 2.0, 0.9 Hz, 1H), 5.44 (ddt, J=7.3, 4.7, 2.3 Hz, 1H) and 5.35 (tt, J=7.4, 1.4 Hz), 5.23-5.13 (m, 2H), 3.75-3.66 (m, 1H), 3.34 (d, J=5.3 Hz, 3H), 3.02 (dd, J=15.9, 7.5 Hz) and 2.91 (dd, J=16.6, 4.8 Hz, 1H), 2.80 (dd, J=16.6, 7.5 Hz, 1H) and 2.74 (dd, J=15.9, 7.4 Hz), 2.03-1.94 (m) and 1.92-1.76 (m, 3H), 1.69-1.50 (m, 3H), 1.48-1.34 (m, 2H), 0.90 (t, J=7.3 Hz, 3H); ¹³C NMR (151 MHz, CDCl₃) δ 176.6, 168.8, 142.9, 134.5, 127.8, 127.6, 127.5, 119.6, 75.4, 70.5, 66.1, 55.7, 44.9, 37.2, 36.1, 21.8, 21.5, 17.0, 13.4; HRMS (ESI-TOF) m/z Calcd for C₂₁H₂₇O₅⁺ [M+H]⁺ 359.1858. found 359.1860.

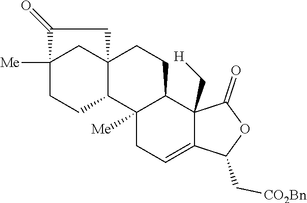 [SMILES: C[C@@]12CC[C@@H]3[C@@](CC[C@H]4[C@]5(C)C(=O)O[C@H](CC(=O)OCc6ccccc6)C5=CC[C@@]34C)(CC1=O)C2]

### Benzyl 2-((3aS,3bS,5aR,8S,10aR,10bS)-3a,8,10b-trimethyl-3,7-dioxo-1,3,3a,3b,4,5,6,7,8,9,10,10a,10b,11-tetradecahydro-5a,8-methanocyclohepta[5,6]naphtho[1,2-c]furan-1-yl)acetate (3r)

Following General Procedure on 0.1 mmol scale. The product was obtained as colorless oil (44% yield). ¹H NMR (600 MHz, Chloroform-d) δ 7.41-7.31 (m, 5H), 5.61 (dt, J=8.1, 2.1 Hz, 1H), 5.41 (tdd, J=5.1, 3.1, 1.8 Hz, 1H), 5.18 (d, J=2.0 Hz, 2H), 2.86 (dd, J=16.4, 5.0 Hz, 1H), 2.78 (dd, J=16.3, 7.6 Hz, 1H), 2.64 (dd, J=18.7, 3.8 Hz, 1H), 2.14 (dd, J=16.4, 8.1 Hz, 1H), 2.02 (dq, J=14.4, 3.3 Hz, 1H), 1.93-1.82 (m, 2H), 1.76 (dt, J=13.4, 3.2 Hz, 1H), 1.69-1.58 (m, 4H), 1.55-1.36 (m, 8H), 1.34-1.27 (m, 1H), 0.99 (s, 3H), 0.64 (s, 3H); ¹³C NMR (151 MHz, CDCl₃) δ 222.0, 175.8, 169.9, 140.5, 135.5, 128.8, 128.6, 128.5, 120.3, 74.7, 67.2, 54.1, 53.5, 52.9, 48.8, 48.7, 43.8, 41.0, 39.5, 38.2, 37.8, 37.2, 37.0, 25.1, 21.0, 20.3, 20.0, 13.0; HRMS (ESI-TOF) m/z Calcd for C₃₀H₃₇O₅⁺ [M+H]⁺ 519.2747. found 519.2751.

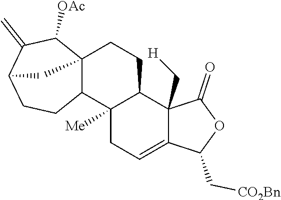

### Benzyl 2-((1S,3aR,10aS,10bS)-6-acetoxy-3a,10b-dimethyl-7-methylene-3-oxo-1,3,3a,3b,4,5,6,7,8,9,10,10a,10b,11-tetradecahydro-5a,8-methanocyclohepta[5,6]naphtho[1,2-c]furan-1-yl)acetate (3s)

Following General Procedure on 0.1 mmol scale. The product was obtained as colorless oil (40% yield). ¹H NMR (600 MHz, Chloroform-d) δ 7.41-7.30 (m, 5H), 5.63 (ddt, J=13.2, 8.1, 2.2 Hz, 1H), 5.42 (dddd, J=8.0, 5.1, 3.3, 1.9 Hz, 1H), 5.18 (t, J=2.1 Hz, 3H), 4.95 (dd, J=3.0, 1.0 Hz, 1H), 4.89 (dd, J=2.4, 1.2 Hz, 1H), 2.88 (dd, J=16.3, 4.9 Hz, 1H), 2.82-2.75 (m, 1H), 2.69 (s, 1H) and 2.39 (s), 2.32 (dd, J=16.5, 8.2 Hz, 1H) and 2.26-2.21 (m), 2.17 (d, J=4.4 Hz, 3H), 2.05 (d, J=12.0 Hz, 1H), 1.98-1.86 (m, 2H), 1.84-1.74 (m, 1H), 1.69-1.58 (m, 2H), 1.55-1.46 (m, 3H), 1.43 (d, J=1.5 Hz, 3H), 1.42-1.31 (m, 3H), 0.82 (s, 3H), 1.24-1.19 (m, 1H); ¹³C NMR (151 MHz, CDCl₃) δ 174.9, 170.4, 168.9, 152.4, 139.4, 134.5, 127.8, 127.62, 127.57, 119.7, 105.6, 80.6, 73.8, 66.1, 51.8, 45.0, 44.0, 43.1, 39.5, 38.4, 37.4, 37.3, 36.8, 35.5, 32.1, 23.8, 20.5, 18.9, 17.8, 14.3; HRMS (ESI-TOF) m/z Calcd for C₃₂H₃₉O₆⁺ [M+H]⁺ 519.2747. found 519.2751.

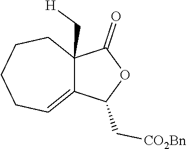 [SMILES: C[C@]12CCCCC=C1[C@@H](CC(=O)OCc1ccccc1)OC2=O]

### Benzyl 2-((3aS)-3a-methyl-3-oxo-3,3a,4,5,6,7-hexahydro-1H-cyclohepta[c]furan-1-yl)acetate (3t)

Following General Procedure on 0.1 mmol scale. The product was obtained as colorless oil (82% yield). ¹H NMR (600 MHz, Chloroform-d) δ 7.40-7.30 (m, 5H), 5.76 (ddd, J=7.8, 5.7, 1.7 Hz) and 5.67 (ddd, J=8.1, 4.5, 2.3 Hz, 1H), 5.35-5.24 (m, 1H), 5.21-5.12 (m, 2H), 2.92-2.78 (m, 1H), 2.77-2.63 (m, 1H), 2.23-2.11 (m, 2H), 2.01-1.74 (m, 4H), 1.54 (tdd, J=15.1, 9.1, 3.8 Hz, 1H), 1.40 (d, J=18.9 Hz, 3H), 1.32-1.20 (m, 1H); ¹³C NMR (151 MHz, CDCl₃) δ 181.2 and 180.7, 169.7 and 169.5, 144.7 and 144.0, 135.6 and 135.5, 128.8 and 128.7, 128.6 and 128.6, 128.5, 126.1 and 124.2, 77.1 and 76.0, 67.0 and 66.9, 47.2 and 46.2, 42.9 and 39.9, 35.0 and 34.3, 28.0 and 27.9, 27.4 and 27.3, 25.4 and 25.2, 21.2 and 20.3; HRMS (ESI-TOF) m/z Calcd for C₁₉H₂₃O₄⁺ [M+H]⁺ 315.1596. found 315.1597.

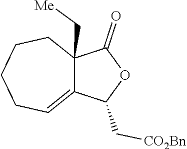 [SMILES: CC[C@]12CCCCC=C1[C@@H](CC(=O)OCc1ccccc1)OC2=O]

### Benzyl 2-((3aS)-3a-ethyl-3-oxo-3,3a,4,5,6,7-hexahydro-1H-cyclohepta[c]furan-1-yl)acetate (3u)

Following General Procedure on 0.1 mmol scale. The product was obtained as colorless oil (62% yield). ¹H NMR (600 MHz, Chloroform-d) δ 7.40-7.29 (m, 5H), 5.78-5.72 (m, 1H), 5.22 (ddq, J=7.1, 3.3, 1.7 Hz, 1H), 5.16 (s, 2H), 2.87 (dd, J=16.6, 4.0 Hz, 1H), 2.73 (dd, J=16.6, 7.1 Hz, 1H), 2.23-2.14 (m, 2H), 2.08 (dq, J=13.5, 7.5 Hz, 1H), 2.01 (dddd, J=13.9, 4.9, 2.7, 1.0 Hz, 1H), 1.91-1.74 (m, 3H), 1.66 (dq, J=13.5, 7.4 Hz, 1H), 1.54 (ddd, J=13.9, 12.8, 3.3 Hz, 1H), 1.24 (dddd, J=18.1, 13.6, 11.3, 3.4 Hz, 1H), 0.93 (t, J=7.5 Hz, 3H); 13C NMR (151 MHz, CDCl₃) δ 179.1, 168.7, 143.2, 134.6, 127.7, 127.5, 123.7, 75.9, 65.9, 51.2, 39.5, 32.3, 26.9, 26.2, 25.7, 24.4, 8.3; HRMS (ESI-TOF) m/z Calcd for C₂₀H₂₅O₄⁺ [M+H]⁺+329.1753. found 329.1758.

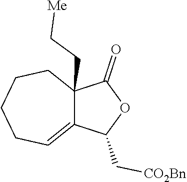 [SMILES: CCC[C@]12CCCCC=C1[C@@H](CC(=O)OCc1ccccc1)OC2=O]

### Benzyl 2-((3aS)-3-oxo-3a-propyl-3,3a,4,5,6,7-hexahydro-1H-cyclohepta[c]furan-1-yl)acetate (3v)

Following General Procedure on 0.1 mmol scale. The product was obtained as colorless oil (70% yield). ¹H NMR (600 MHz, Chloroform-d) δ 7.39-7.31 (m, 5H), 5.73 (ddd, J=8.5, 4.6, 2.3 Hz, 1H), 5.22 (ddt, J=6.7, 3.8, 2.6 Hz, 1H), 5.16 (s, 2H), 2.86 (dd, J=16.6, 4.1 Hz, 1H), 2.73 (dd, J=16.6, 7.1 Hz, 1H), 2.26-2.11 (m, 2H), 2.05-1.95 (m, 2H), 1.92-1.73 (m, 3H), 1.60-1.50 (m, 2H), 1.41-1.14 (m, 3H), 0.92 (t, J=7.3 Hz, 3H); ¹³C NMR (151 MHz, CDCl₃) δ 179.3, 168.7, 143.4, 134.6, 127.78, 127.74, 127.61, 127.55, 123.6, 75.9, 65.9, 50.8, 39.5, 35.0, 32.8, 26.9, 26.2, 24.4, 17.2, 13.5; HRMS (ESI-TOF) m/z Calcd for C₂₁H₂₇O₄⁺ [M+H]⁺ 343.1909. found 343.1914.

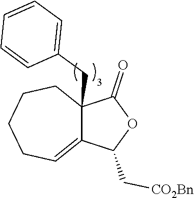

### Benzyl 2-((3aR)-3-oxo-3a-(3-phenylpropyl)-3,3a,4,5,6,7-hexahydro-1H-cyclohepta[c]furan-1-yl)acetate (3w)

Following General Procedure on 0.1 mmol scale. The product was obtained as colorless oil (60% yield). ¹H NMR (600 MHz, Chloroform-d) δ 7.41-7.30 (m, 5H), 7.29-7.26 (m, 2H), 7.20-7.12 (m, 3H), 5.74 (dddd, J=28.6, 8.8, 4.6, 2.1 Hz, 1H), 5.31 (dq, J=6.7, 2.5 Hz) and 5.20 (ddq, J=6.8, 4.4, 2.6 Hz, 1H), 5.15 (s, 2H), 2.85 (dd, J=16.6, 4.1 Hz, 1H) and 2.78 (dd, J=16.1, 8.7 Hz), 2.74-2.53 (m, 3H), 2.17-1.81 (m, 5H), 1.81-1.58 (m, 5H), 1.55-1.45 (m, 1H), 1.25-1.16 (m, 1H); ¹³C NMR (151 MHz, CDCl₃) δ 179.5, 169.0, 143.6, 141.0, 135.0, 128.1, 127.95, 127.90, 127.88, 125.5, 124.2, 76.2, 66.3, 50.9, 39.9, 35.5, 33.0, 32.6, 27.2, 26.5, 25.8, 24.8; HRMS (ESI-TOF) m/z Calcd for C₂₇H₃₁O₄⁺ [M+H]⁺ 419.2222. found 419.2222.

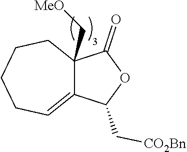

### Benzyl 2-((3aR)-3a-(3-methoxypropyl)-3-oxo-3,3a,4,5,6,7-hexahydro-1H-cyclohepta[c]furan-1-yl)acetate (3×)

Following General Procedure on 0.1 mmol scale. The product was obtained as colorless oil (78% yield). ¹H NMR (600 MHz, Chloroform-d) δ 7.40-7.30 (m, 5H), 5.74 (ddd, J=8.8, 4.3, 2.3 Hz, 1H), 5.23 (ddd, J=6.7, 4.0, 2.1 Hz, 1H), 5.15 (s, 2H), 3.42-3.33 (m, 2H), 3.30 (s, 3H), 2.87 (dd, J=16.6, 4.0 Hz, 1H), 2.73 (dd, J=16.6, 7.0 Hz, 1H), 2.27-2.07 (m, 3H), 2.06-1.98 (m, 1H), 1.92-1.74 (m, 3H), 1.72-1.50 (m, 5H); ¹³C NMR (151 MHz, CDCl₃) δ 180.0, 169.6, 144.1, 135.6, 128.7, 128.6, 128.5, 125.0, 76.8, 66.9, 58.6, 51.3, 40.5, 33.5, 30.3, 27.8, 27.1, 25.4, 24.9; HRMS (ESI-TOF) m/z Calcd for C₂₂H₂₉O₅⁺ [M+H]⁺ 373.2015. found 373.2017.

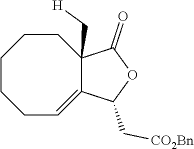 [SMILES: C[C@]12CCCCC/C=C\1[C@@H](CC(=O)OCc1ccccc1)OC2=O]

### Benzyl 2-((1S,E)-3a-methyl-3-oxo-1,3,3a,4,5,6,7,8-octahydrocycloocta[c]furan-1-yl)acetate (3y)

Following General Procedure on 0.1 mmol scale. The product was obtained as colorless oil (55% yield). ¹H NMR (600 MHz, Chloroform-d) δ 7.40-7.31 (m, 5H), 5.48 (dddd, J=10.0, 7.5, 3.6, 2.0 Hz, 1H), 5.41-5.32 (m, 1H), 5.21-5.13 (m, 2H), 2.87-2.74 (m, 2H), 2.42-2.31 (m, 1H), 2.06 (dh, J=13.8, 4.0 Hz, 1H), 1.97-1.75 (m, 2H), 1.75-1.58 (m, 2H), 1.56-1.28 (m, 7H); ¹³C NMR (151 MHz, CDCl₃) δ 181.7 and 181.1, 169.7 and 169.6, 141.7 and 141.5, 135.6 and 135.5, 128.77 and 128.75, 128.59 and 128.56, 124.1 and 123.8, 76.90 and 76.87, 67.1 and 66.9, 46.6 and 46.3, 41.5 and 41.4, 40.5 and 39.6, 27.9 and 27.4, 26.7 and 26.6, 24.0 and 23.9, 23.0 and 22.18, 22.21 and 21.7; HRMS (ESI-TOF) m/z Calcd for C₂₀H₂₅O₄⁺ [M+H]⁺ 329.1753. found 329.1743.

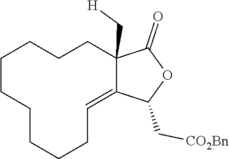 [SMILES: C[C@]12CCCCCCCCC/C=C/1[C@@H](CC(=O)OCc1ccccc1)OC2=O]

### Benzyl (Z)-2-(3a-methyl-3-oxo-1,3,3a,4,5,6,7,8,9,10,11,12-dodecahydrocyclododeca[c]furan-1-yl)acetate (3z)

Following General Procedure on 0.1 mmol scale. The product was obtained as colorless oil (45% yield). ¹H NMR (600 MHz, Chloroform-d) δ 7.41-7.31 (m, 5H), 5.52 (qd, J=7.4, 6.4, 2.7 Hz, 2H), 5.23-5.10 (m, 2H), 2.90 (dd, J=16.6, 2.7 Hz, 1H), 2.66 (dd, J=16.6, 8.8 Hz, 1H), 2.08 (q, J=6.9, 6.3 Hz, 2H), 1.81 (ddd, J=13.3, 8.3, 6.5 Hz, 1H), 1.61-1.48 (m, 6H), 1.35-1.21 (m, 12H); ¹³C NMR (151 MHz, CDCl₃) δ 180.8, 169.5, 141.4, 135.6, 128.8, 128.59, 128.57, 124.7, 76.8, 67.1, 47.3, 40.9, 39.5, 26.8, 26.4, 26.1, 26.0, 25.5, 25.2, 24.2, 22.9, 22.1; HRMS (ESI-TOF) m/z Calcd for C₂₄H₃₃O₄⁺ [M+H]⁺ 385.2379. found 385.2370.

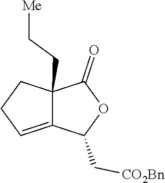 [SMILES: CCC[C@]12CCC=C1[C@@H](CC(=O)OCc1ccccc1)OC2=O]

### Benzyl 2-((3aS)-3-oxo-3a-propyl-3,3a,4,5-tetrahydro-1H-cyclopenta[c]furan-1-yl)acetate (3aa)

Following General Procedure on 0.1 mmol scale. The product was obtained as colorless oil (20% yield). ¹H NMR (600 MHz, Chloroform-d) δ 7.41-7.31 (m, 5H), 5.54 (dddd, J=9.4, 3.2, 2.2, 1.2 Hz, 1H), 5.47 (qd, J=7.1, 2.2 Hz, 1H), 5.22-5.13 (m, 2H), 2.81 (dd, J=16.2, 3.1 Hz, 1H), 2.68 (dd, J=16.2, 9.5 Hz, 1H), 2.26-2.11 (m, 3H), 1.97-1.85 (m, 2H), 1.82-1.70 (m, 3H), 1.65 (dd, J=7.1, 1.2 Hz, 3H); ¹³C NMR (151 MHz, CDCl₃) δ 182.1, 169.5, 144.3, 135.5, 128.8, 128.57, 128.55, 117.9, 76.2, 67.1, 51.7, 42.6, 40.5, 39.4, 26.7, 26.4, 13.8; HRMS (ESI-TOF) m/z Calcd for C₁₉H₂₃O₄⁺ [M+H]⁺ 315.1596. found 315.1593.

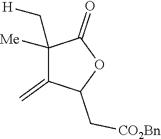 [SMILES: C=C1C(CC(=O)OCc2ccccc2)OC(=O)C1(C)C]

### Benzyl(S)-2-(4,4-dimethyl-3-methylene-5-oxotetrahydrofuran-2-yl)acetate (5a)

Following General Procedure on 0.1 mmol scale. The product was obtained as colorless oil (62% yield). ¹H NMR (600 MHz, Chloroform-d) δ 7.36 (tdd, J=8.3, 6.5, 2.2 Hz, 5H), 5.44 (ddt, J=7.2, 4.7, 2.3 Hz, 1H), 5.17 (d, J=2.1 Hz, 2H), 5.07 (ddd, J=15.3, 2.3, 1.3 Hz, 2H), 2.91-2.78 (m, 2H), 1.34 (d, J=1.3 Hz, 6H); ¹³C NMR (151 MHz, CDCl₃) δ 179.3, 168.4, 151.7, 134.5, 127.8, 127.61, 127.58, 106.3, 75.7, 66.1, 42.1, 39.2, 25.9, 23.9; HRMS (ESI-TOF) m/z Calcd for C₁₆H₁₈O₄⁺ [M+H]⁺ 275.1283. found 275.1282.

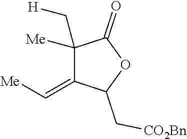 [SMILES: C/C=C1/C(CC(=O)OCc2ccccc2)OC(=O)C1(C)C]

### Benzyl (S,E)-2-(3-ethylidene-4,4-dimethyl-5-oxotetrahydrofuran-2-yl)acetate (5b)

Following General Procedure on 0.1 mmol scale. The product was obtained as colorless oil (40% yield). ¹H NMR (600 MHz, Chloroform-d) δ 7.39-7.32 (m, 5H), 5.57 (dtt, J=9.3, 2.6, 1.3 Hz, 1H), 5.48 (qd, J=7.1, 2.3 Hz, 1H), 5.17 (q, J=12.2 Hz, 2H), 2.87 (dd, J=16.4, 2.9 Hz, 1H), 2.69 (dd, J=16.4, 9.5 Hz, 1H), 1.66 (dd, J=7.1, 1.3 Hz, 3H), 1.30 (d, J=6.4 Hz, 6H); ¹³C NMR (151 MHz, CDCl₃) δ 181.2, 169.4, 142.9, 135.5, 128.8, 128.58, 128.55, 118.8, 76.2, 67.1, 42.4, 40.6, 27.8, 26.5, 13.9; HRMS (ESI-TOF) m/z Calcd for C₁₇H₂₁O₄⁺ [M+H]⁺ 289.1440. found 289.1439.

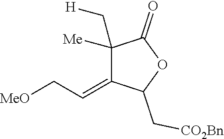 [SMILES: COC/C=C1/C(CC(=O)OCc2ccccc2)OC(=O)C1(C)C]

### Benzyl (S,E)-2-(3-(2-methoxyethylidene)-4,4-dimethyl-5-oxotetrahydrofuran-2-yl)acetate (5c)

Following General Procedure on 0.1 mmol scale. The product was obtained as colorless oil (38% yield). ¹H NMR (600 MHz, Chloroform-d) δ 7.40-7.32 (m, 5H), 5.65 (dtt, J=9.0, 2.4, 1.1 Hz, 1H), 5.54 (td, J=6.1, 2.3 Hz, 1H), 5.21-5.13 (m, 2H), 3.99-3.88 (m, 2H), 3.32 (s, 3H), 2.92 (dd, J=16.6, 3.0 Hz, 1H), 2.73 (dd, J=16.6, 9.0 Hz, 1H), 1.34 (d, J=10.4 Hz, 6H); ¹³C NMR (151 MHz, CDCl₃) δ 180.5, 169.4, 146.1, 135.5, 128.8, 128.6, 128.5, 120.2, 76.2, 68.7, 67.1, 58.7, 42.8, 41.3, 27.4, 26.6; HRMS (ESI-TOF) m/z Calcd for C₁₈H₂₃O₅⁺ [M+H]⁺ 319.1545. found 319.1555.

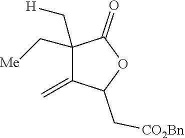 [SMILES: C=C1C(CC(=O)OCc2ccccc2)OC(=O)C1(C)CC]

### Benzyl 2-((2S)-4-ethyl-4-methyl-3-methylene-5-oxotetrahydrofuran-2-yl)acetate (5d)

Following General Procedure on 0.1 mmol scale. The product was obtained as colorless oil (46% yield). ¹H NMR (600 MHz, Chloroform-d) δ 7.44-7.32 (m, 5H), 5.50 (ddt, J=7.1, 5.9, 2.4 Hz) amd 5.36 (ddt, J=7.1, 4.6, 2.4 Hz, 1H), 5.26-5.13 (m, 3H), 5.06 (td, J=2.4, 1.1 Hz, 1H), 2.91 (dd, J=16.6, 4.5 Hz) and 2.85-2.78 (m, 2H), 1.91-1.78 (m, 1H), 1.67-1.58 (m, 1H), 1.34 (d, J=10.1 Hz, 3H), 0.88 (dt, J=25.5, 7.4 Hz, 3H); ¹³C NMR (151 MHz, CDCl₃) δ 180.0 and 179.8, 169.6 and 169.4, 151.3 and 150.5, 135.48 and 135.45, 128.78 and 128.76, 128.63 and 128.60, 128.60 and 128.57, 108.2 and 107.8, 77.3 and 76.8, 67.2 and 67.1, 48.0 and 47.9, 40.6 and 40.4, 33.6 and 31.5, 26.1 and 23.7, 9.5 and 9.1; HRMS (ESI-TOF) m/z Calcd for C₁₇H₂₁O₄⁺ [M+H]⁺ 289.1440. found 289.1440.

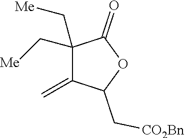 [SMILES: C=C1C(CC(=O)OCc2ccccc2)OC(=O)C1(CC)CC]

### Benzyl(S)-2-(4,4-diethyl-3-methylene-5-oxotetrahydrofuran-2-yl)acetate (5e)

Following General Procedure on 0.1 mmol scale. The product was obtained as colorless oil (74% yield). ¹H NMR (600 MHz, Chloroform-d) δ 7.44-7.31 (m, 5H), 5.38 (ddt, J=7.5, 4.8, 2.4 Hz, 1H), 5.24-5.15 (m, 3H), 5.01 (dd, J=2.7, 1.2 Hz, 1H), 2.85-2.70 (m, 2H), 1.91-1.79 (m, 2H), 1.62-1.55 (m, 2H), 0.84 (dt, J=33.8, 7.4 Hz, 6H); ¹³C NMR (151 MHz, CDCl₃) § 179.6, 169.7, 149.0, 135.5, 128.8, 128.62, 128.59, 108.5, 77.6, 67.2, 53.7, 40.7, 33.5, 30.7, 9.6, 9.1; HRMS (ESI-TOF) m/z Calcd for C₁₈H₂₂O₄⁺ [M+H]⁺ 303.1596. found 303.1591.

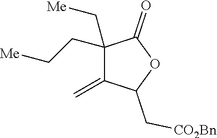 [SMILES: C=C1C(CC(=O)OCc2ccccc2)OC(=O)C1(CC)CCC]

### Benzyl 2-((2S)-4-ethyl-3-methylene-5-oxo-4-propyltetrahydrofuran-2-yl)acetate (5f)

Following General Procedure on 0.1 mmol scale. The product was obtained as colorless oil (54% yield). ¹H NMR (600 MHz, Chloroform-d) δ 7.43-7.34 (m, 5H), 5.40 (ddtd, J=8.7, 5.0, 2.5, 1.3 Hz, 1H), 5.25-5.17 (m, 3H), 5.03 (dt, J=2.6, 1.2 Hz, 1H), 2.85-2.74 (m, 2H), 1.93-1.73 (m, 2H), 1.64-1.58 (m, 1H), 1.52 (tdd, J=13.0, 7.7, 4.3 Hz, 1H), 1.39-1.29 (m, 1H), 1.27-1.10 (m, 1H), 0.93-0.81 (m, 6H); ¹³C NMR (151 MHz, CDCl₃) δ 178.7, 168.7, 148.5 and 148.4, 134.5, 127.8, 127.6, 127.6, 107.3, 76.6 and 76.5, 66.2, 52.2 and 52.1, 41.9 and 39.8, 39.7 and 39.2, 32.8 and 30.1, 17.6 and 17.1, 13.4 and 13.3, 8.5 and 8.0; HRMS (ESI-TOF) m/z Calcd for C₁₉H₂₅O₄⁺ [M+H]⁺ 317.1753. found 317.1760.

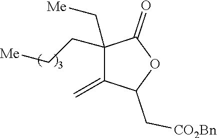

### Benzyl 2-((2S)-4-ethyl-3-methylene-5-oxo-4-pentyltetrahydrofuran-2-yl)acetate (5g)

Following General Procedure on 0.1 mmol scale. The product was obtained as colorless oil (52% yield). ¹H NMR (600 MHz, Chloroform-d) δ 7.41-7.31 (m, 5H), 5.38 (dtt, J=7.8, 5.0, 2.4 Hz, 1H), 5.23-5.15 (m, 3H), 5.01 (dd, J=2.7, 1.1 Hz, 1H), 2.83-2.70 (m, 2H), 1.91-1.72 (m, 2H), 1.60-1.46 (m, 3H), 1.32-1.06 (m, 5H), 0.90-0.79 (m, 6H); ¹³C NMR (151 MHz, CDCl₃) δ 179.7, 169.7, 149.5 and 149.4, 135.5, 128.8, 128.64, 128.61, 108.3, 77.6 and 77.5, 67.2, 53.2 and 53.1, 40.8 and 40.7, 40.7 and 37.9, 33.8 and 31.2, 32.03 and 31.99, 24.8 and 24.4, 22.50 and 22.46, 14.1, 9.6 and 9.0; HRMS (ESI-TOF) m/z Calcd for C₂₁H₂₉O₄⁺ [M+H]⁺ 345.2066. found 345.2065.

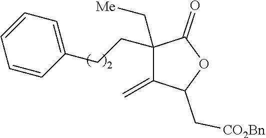

### Benzyl 2-((2S)-4-ethyl-3-methylene-5-oxo-4-(3-phenylpropyl)tetrahydrofuran-2-yl)acetate (5h)

Following General Procedure on 0.1 mmol scale. The product was obtained as colorless oil (42% yield). ¹H NMR (600 MHz, Chloroform-d) δ 7.40-7.32 (m, 5H), 7.29-7.24 (m, 2H), 7.17 (td, J=7.3, 5.2 Hz, 1H), 7.14-7.09 (m, 2H), 5.39-5.33 (m, 1H), 5.22-5.14 (m, 3H), 4.95 (ddd, J=23.0, 2.7, 1.2 Hz, 1H), 2.80-2.69 (m, 2H), 2.62 (tdd, J=10.6, 8.6, 5.2 Hz, 1H), 2.52 (ddd, J=13.9, 8.6, 6.4 Hz, 1H), 1.89-1.76 (m, 2H), 1.65-1.42 (m, 4H), 0.83 (dt, J=32.5, 7.4 Hz, 3H); ¹³C NMR (151 MHz, CDCl₃) δ 179.5, 169.61 and 169.59, 149.2 and 149.1, 141.6, 135.5, 128.8, 128.63 and 128.61, 128.6, 128.51 and 128.49, 128.47 and 128.46, 126.1 and 126.0, 108.6 and 108.5, 77.6 and 77.5, 67.2, 53.0 and 52.9, 40.7 and 40.1, 37.1 and 36.0, 33.7 and 31.1, 26.6 and 26.4, 9.5 and 9.0; HRMS (ESI-TOF) m/z Calcd for C C₂₅H₂₉O₄⁺ [M+H]⁺ 393.2066. found 393.2069.

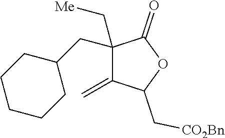 [SMILES: C=C1C(CC(=O)OCc2ccccc2)OC(=O)C1(CC)CC1CCCCC1]

### Benzyl 2-((2S)-4-(cyclohexylmethyl)-4-ethyl-3-methylene-5-oxotetrahydrofuran-2-yl)acetate (5i)

Following General Procedure on 0.1 mmol scale. The product was obtained as colorless oil (34% yield). ¹H NMR (600 MHz, Chloroform-d) δ 7.41-7.32 (m, 5H), 5.45-5.36 (m, 1H), 5.24-5.16 (m, 3H), 5.00 (ddd, J=8.4, 2.7, 1.2 Hz, 1H), 2.84-2.72 (m, 2H), 1.86-1.72 (m, 3H), 1.67-1.58 (m, 2H), 1.51 (dddd, J=18.3, 13.9, 10.7, 5.9 Hz, 3H), 1.34-1.23 (m, 2H), 1.21-1.03 (m, 3H), 1.00-0.87 (m, 2H), 0.82 (dt, J=33.0, 7.4 Hz, 3H); ¹³C NMR (151 MHz, CDCl₃) δ 180.0, 169.61 and 169.59, 149.1, 135.4 and 135.3, 128.6, 128.49 and 128.48, 128.4, 108.6 and 108.5, 77.4 and 77.3, 67.0, 51.8, 47.6, 44.7, 40.66 and 40.60, 35.4 and 35.1, 34.5 and 33.6, 33.25 and 33.20, 26.3 and 26.1, 26.1 and 26.0, 26.0, 9.2 and 8.7; HRMS (ESI-TOF) m/z Calcd for C₂₃H₃₀O₄⁺ [M+H]⁺ 371.2222. found 371.2221.

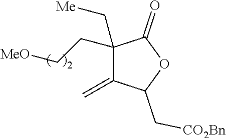

### Benzyl 2-((2S)-4-ethyl-4-(3-methoxypropyl)-3-methylene-5-oxotetrahydrofuran-2-yl)acetate (5j)

Following General Procedure on 0.1 mmol scale. The product was obtained as colorless oil (58% yield). ¹H NMR (600 MHz, Chloroform-d) δ 7.42-7.31 (m, 5H), 5.38 (dddd, J=10.7, 7.0, 4.8, 2.4 Hz, 1H), 5.22 (s, 1H), 5.21-5.15 (m, 2H), 5.04 (dt, J=2.6, 1.2 Hz, 1H), 3.38-3.26 (m, 5H), 2.84-2.71 (m, 2H), 1.90-1.77 (m, 2H), 1.72-1.33 (m, 4H), 0.85 (dt, J=34.7, 7.4 Hz, 3H); ¹³C NMR (151 MHz, CDCl₃) δ 179.44 and 179.42, 169.64 and 169.58, 149.04 and 148.95, 135.4, 128.8, 128.63 and 128.62, 128.60 and 128.58, 108.8 and 108.7, 77.6 and 77.5, 72.4 and 72.3, 67.2, 58.59 and 58.57, 52.69 and 52.67, 40.7 and 40.6, 37.0 and 34.2, 33.8 and 31.1, 25.3 and 24.9, 9.5 and 9.0; HRMS (ESI-TOF) m/z Calcd for C₂₀H₂₆O₅⁺ [M+H]⁺ 347.1858. found 347.1861.

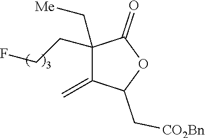

### Benzyl 2-((2S)-4-ethyl-4-(4-fluorobutyl)-3-methylene-5-oxotetrahydrofuran-2-yl)acetate (5k)

Following General Procedure on 0.1 mmol scale. The product was obtained as colorless oil (44% yield). ¹H NMR (600 MHz, Chloroform-d) δ 7.41-7.32 (m, 5H), 5.38 (tdd, J=8.3, 4.8, 2.5 Hz, 1H), 5.23 (dt, J=3.3, 1.6 Hz, 1H), 5.22-5.15 (m, 2H), 5.03 (td, J=2.8, 1.2 Hz, 1H), 4.50-4.29 (m, 2H), 2.84-2.71 (m, 2H), 1.92-1.76 (m, 2H), 1.74-1.52 (m, 3H), 1.49-1.16 (m, 3H), 0.85 (dt, J=35.1, 7.4 Hz, 3H); ¹³C NMR (151 MHz, CDCl₃) δ 179.43 and 179.40, 169.60 and 169.58, 149.1 and 149.0, 135.4, 128.8, 128.65, 128.61, 108.8 and 108.6, 84.3 and 83.2, 77.6 and 77.5, 67.2, 52.99 and 52.95, 40.68 and 40.65, 40.1 and 37.2, 33.8 and 31.2, 30.6 (dd, J=19.8, 6.0 Hz), 20.9 (dd, J=52.3, 5.1 Hz), 9.5 and 9.0; HRMS (ESI-TOF) m/z Calcd for C₂₀H₂₆FO₄⁺ [M+H]⁺ 349.1815. found 349.1819.

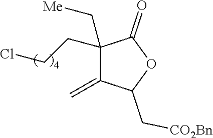

### Benzyl 2-((2S)-4-(5-chloropentyl)-4-ethyl-3-methylene-5-oxotetrahydrofuran-2-yl)acetate (51)

Following General Procedure on 0.1 mmol scale. The product was obtained as colorless oil (42% yield). ¹H NMR (600 MHz, Chloroform-d) δ 7.39-7.33 (m, 5H), 5.38 (dddd, J=9.6, 4.7, 3.8, 2.5 Hz, 1H), 5.22 (ddd, J=2.9, 2.2, 0.9 Hz, 1H), 5.20-5.16 (m, 2H), 5.02 (ddd, J=3.9, 2.7, 1.2 Hz, 1H), 3.49 (tdd, J=6.7, 3.3, 0.8 Hz, 2H), 2.82-2.71 (m, 2H), 1.91-1.69 (m, 4H), 1.63-1.49 (m, 2H), 1.40 (dtdd, J=9.2, 7.5, 6.5, 1.4 Hz, 2H), 1.36-1.07 (m, 2H), 0.84 (dt, J=34.8, 7.4 Hz, 3H); ¹³C NMR (151 MHz, CDCl₃) δ 178.49 and 178.47, 168.63 and 168.58, 148.3 and 148.2, 134.4, 127.80 and 127.79, 127.7 and 127.6, 127.62 and 127.61, 107.6 and 107.5, 76.6 and 76.5, 66.21 and 66.21, 52.01 and 51.96, 44.02 and 43.98, 39.7 and 39.4, 36.5 and 32.8, 31.34 and 31.33, 30.2, 26.1 and 26.0, 23.5 and 23.1, 8.5 and 8.0; HRMS (ESI-TOF) m/z Calcd for C₂₁H₂₈ClO₄⁺ [M+H]⁺ 379.1676. found 379.1673.

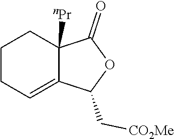

### Methyl 2-((3aS)-3-oxo-3a-propyl-1,3,3a,4,5,6-hexahydroisobenzofuran-1-yl)acetate (6a)

Following General Procedure on 0.1 mmol scale. The product was obtained as colorless oil (84% yield). ¹H NMR (600 MHz, Chloroform-d) δ 5.60 (tt, J=3.4, 1.6 Hz, 1H), 5.40 (qd, J=5.1, 4.5, 2.0 Hz, 1H), 3.73 (d, J=1.3 Hz, 3H), 2.87-2.79 (m, 1H), 2.66 (ddd, J=16.3, 7.8, 1.4 Hz, 1H), 2.18 (ddtd, J=15.7, 6.9, 3.5, 1.8 Hz, 1H), 2.05 (ddt, J=17.2, 13.6, 3.8 Hz, 2H), 1.75-1.61 (m, 4H), 1.50-1.30 (m, 3H), 0.90 (td, J=7.3, 1.4 Hz, 3H); ¹³C NMR (151 MHz, CDCl₃) δ 178.8, 170.5, 139.8, 120.9, 77.0, 52.2, 45.3, 38.3, 38.2, 26.6, 23.8, 17.9, 16.9, 14.4; HRMS (ESI-TOF) m/z Calcd for C₁₄H₂₁O₄⁺ [M+H]⁺ 253.1440. found 253.1434.

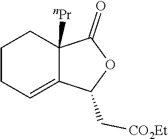

### Ethyl 2-((3aS)-3-oxo-3a-propyl-1,3,3a,4,5,6-hexahydroisobenzofuran-1-yl)acetate (6b)

Following General Procedure on 0.1 mmol scale. The product was obtained as colorless oil (76% yield). ¹H NMR (600 MHz, Chloroform-d) δ 5.61 (dp, J=3.5, 1.6 Hz, 1H), 5.40 (ddq, J=7.6, 5.1, 2.3 Hz, 1H), 4.18 (qt, J=7.2, 1.9 Hz, 2H), 2.81 (ddd, J=16.4, 4.6, 1.4 Hz, 1H), 2.65 (ddd, J=16.3, 7.7, 1.6 Hz, 1H), 2.23-2.14 (m, 1H), 2.11-2.00 (m, 2H), 1.79-1.56 (m, 4H), 1.49-1.30 (m, 3H), 1.27 (td, J=7.2, 1.6 Hz, 3H), 0.90 (td, J=7.3, 1.5 Hz, 3H); ¹³C NMR (151 MHz, CDCl₃) δ 178.8, 170.1, 139.8, 120.8, 77.0, 61.2, 45.3, 38.5, 38.2, 26.6, 23.8, 17.9, 16.9, 14.4, 14.3; HRMS (ESI-TOF) m/z Calcd for C₁₅H₂₃O₄⁺ [M+H]⁺ 267.1596. found 267.1599.

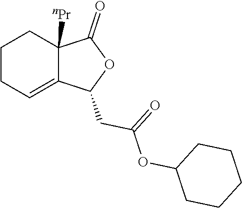

### Cyclohexyl 2-((3aS)-3-oxo-3a-propyl-1,3,3a,4,5,6-hexahydroisobenzofuran-1-yl)acetate (6d)

Following General Procedure on 0.1 mmol scale. The product was obtained as colorless oil (76% yield). ¹H NMR (600 MHz, Chloroform-d) δ 5.61 (td, J=3.6, 2.0 Hz, 1H), 5.44-5.36 (m, 1H), 4.81 (tt, J=9.2, 3.9 Hz, 1H), 2.79 (dd, J=16.3, 4.8 Hz, 1H), 2.66 (dd, J=16.3, 7.4 Hz, 1H), 2.17 (ddtd, J=18.9, 6.8, 3.4, 1.8 Hz, 1H), 2.10-2.00 (m, 2H), 1.91-1.81 (m, 2H), 1.77-1.60 (m, 6H), 1.57-1.50 (m, 1H), 1.49-1.30 (m, 7H), 1.27 (dt, J=9.3, 3.2 Hz, 1H), 0.91 (t, J=7.3 Hz, 3H); ¹³C NMR (151 MHz, CDCl₃) δ 177.9, 168.5, 139.0, 119.8, 72.7, 44.3, 37.8, 37.2, 30.7, 25.6, 24.5, 22.9, 22.8, 16.9, 15.9, 13.5; HRMS (ESI-TOF) m/z Calcd for C₁₉H₂₉O₄⁺ [M+H]⁺ 321.2066. found 321.2064.

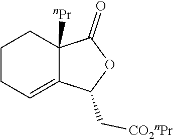

### Propyl 2-((3aS)-3-oxo-3a-propyl-1,3,3a,4,5,6-hexahydroisobenzofuran-1-yl)acetate (6c)

Following General Procedure on 0.1 mmol scale. The product was obtained as colorless oil (76% yield). ¹H NMR (600 MHz, Chloroform-d) δ 5.61 (hept, J=1.6 Hz, 1H), 5.40 (ddq, J=7.5, 4.9, 2.4 Hz, 1H), 4.09 (tt, J=6.7, 2.1 Hz, 2H), 2.82 (ddd, J=16.3, 4.6, 1.3 Hz, 1H), 2.66 (ddd, J=16.3, 7.7, 1.5 Hz, 1H), 2.18 (ddtd, J=15.7, 7.0, 3.5, 1.9 Hz, 1H), 2.11-2.01 (m, 2H), 1.79-1.57 (m, 6H), 1.49-1.30 (m, 3H), 0.92 (dtd, J=23.5, 7.4, 1.5 Hz, 6H); ¹³C NMR (151 MHz, CDCl₃) δ 178.8, 170.2, 139.9, 120.8, 77.1, 66.8, 45.3, 38.4, 38.2, 26.6, 23.8, 22.0, 17.9, 16.9, 14.5, 10.5; HRMS (ESI-TOF) m/z Calcd for C₁₆H₂₅O₄⁺ [M+H]⁺ 281.1753. found 281.1750.

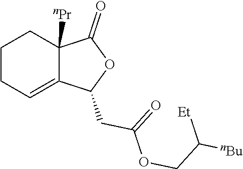

### 2-ethylhexyl 2-((1R,3aS)-3-oxo-3a-propyl-1,3,3a,4,5,6-hexahydroisobenzofuran-1-yl)acetate (6e)

Following General Procedure on 0.1 mmol scale. The product was obtained as colorless oil (78% yield). ¹H NMR (600 MHz, Chloroform-d) δ 5.61 (dt, J=4.4, 2.4 Hz, 1H), 5.43-5.36 (m, 1H), 4.10-3.99 (m, 2H), 2.81 (dd, J=16.3, 4.8 Hz, 1H), 2.68 (dd, J=16.3, 7.5 Hz, 1H), 2.24-1.99 (m, 3H), 1.77-1.55 (m, 5H), 1.49-1.22 (m, 11H), 0.90 (q, J=7.9 Hz, 9H); 13C NMR (151 MHz, CDCl₃) δ 178.8, 170.3, 139.9, 120.8, 77.1, 67.67 and 67.66, 45.3, 38.8, 38.5, 38.2, 30.5, 29.0, 26.7, 23.88 and 23.87, 23.8, 23.1, 17.9, 16.9, 14.5, 14.2, 11.1; HRMS (ESI-TOF) m/z Calcd for C₂₁H₃₅O₄⁺ [M+H]⁺ 351.2535. found 351.2541.

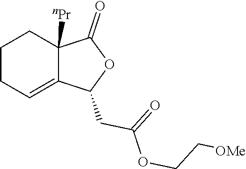

### 2-methoxyethyl 2-((3aS)-3-oxo-3a-propyl-1,3,3a,4,5,6-hexahydroisobenzofuran-1-yl)acetate (6f)

Following General Procedure on 0.1 mmol scale. The product was obtained as colorless oil (84% yield). ¹H NMR (600 MHz, Chloroform-d) δ 5.61 (dp, J=3.6, 1.7 Hz, 1H), 5.44-5.37 (m, 1H), 4.33-4.23 (m, 2H), 3.60 (td, J=4.7, 1.4 Hz, 2H), 3.38 (d, J=1.6 Hz, 3H), 2.87 (ddd, J=16.4, 4.4, 1.4 Hz, 1H), 2.70 (ddd, J=16.5, 7.7, 1.6 Hz, 1H), 2.22-2.13 (m, 1H), 2.10-1.99 (m, 2H), 1.77-1.59 (m, 4H), 1.48-1.30 (m, 3H), 0.90 (td, J=7.3, 1.6 Hz, 3H); 13C NMR (151 MHz, CDCl₃) δ 179.0, 170.1, 139.6, 121.0, 77.0, 70.4, 64.1, 59.1, 45.3, 38.3, 38.2, 26.6, 23.8, 17.9, 16.9, 14.4; HRMS (ESI-TOF) m/z Calcd for C₁₆H₂₅O₅⁺ [M+H]⁺ 297.1702. found 297.1701.

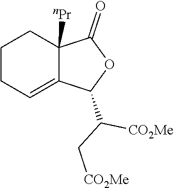

### Dimethyl(2S)-2-((3aS)-3-oxo-3a-propyl-1,3,3a,4,5,6-hexahydroisobenzofuran-1-yl) succinate (6h)

Following General Procedure on 0.1 mmol scale. The product was obtained as colorless oil (59% yield). ¹H NMR (600 MHz, Chloroform-d) δ 5.64 (td, J=3.7, 2.0 Hz, 1H) and 5.61 (td, J=3.7, 2.1 Hz), 5.54 (p, J=2.9 Hz, 1H) and 5.34 (p, J=3.2 Hz), 3.76 (s, 2H) and 3.70 (d, J=5.1 Hz, 4H), 3.57 (ddd, J=10.9, 5.7, 3.5 Hz) and 3.36 (dt, J=10.0, 3.0 Hz, 1H), 2.91-2.77 (m, 1H), 2.55 (dd, J=16.9, 5.7 Hz) and 2.35 (dd, J=17.0, 3.1 Hz, 1H), 2.26-2.00 (m, 3H), 1.79-1.56 (m, 4H), 1.47-1.22 (m, 3H), 0.90 (td, J=7.3, 4.0 Hz, 3H); ¹³C NMR (151 MHz, CDCl₃) δ 178.7 and 178.4, 172.6 and 172.06, 172.11 and 171.2, 137.9 and 137.4, 121.7, 80.1 and 79.1, 52.8, 52.2, 45.7 and 45.3, 43.7 and 43.5, 38.8 and 38.4, 31.3 and 29.9, 26.44 and 26.42, 23.62 and 23.56, 17.8, 16.63 and 16.58, 14.42 and 14.41; HRMS (ESI-TOF) m/z Calcd for C₁₇H₂₅O₆⁺ [M+H]⁺ 325.1651. found 325.1651.

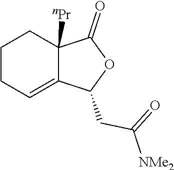

### N,N-dimethyl-2-((3aS)-3-oxo-3a-propyl-1,3,3a,4,5,6-hexahydroisobenzofuran-1-yl) acetamide (6j)

Following General Procedure on 0.1 mmol scale. The product was obtained as colorless oil (54% yield). ¹H NMR (600 MHz, Chloroform-d) § 5.63 (td, J=3.6, 2.0 Hz, 1H), 5.55 (dddd, J=8.0, 5.1, 4.0, 2.5 Hz, 1H), 3.04 (s, 3H), 2.99 (s, 3H), 2.80 (dd, J=15.7, 7.1 Hz, 1H), 2.66 (dd, J=15.7, 5.0 Hz, 1H), 2.23-2.14 (m, 1H), 2.10-1.98 (m, 2H), 1.78-1.59 (m, 4H), 1.49-1.30 (m, 3H), 0.92-0.86 (m, 3H); ¹³C NMR (151 MHz, CDCl₃) δ 178.1, 168.5, 139.5, 119.6, 77.4, 44.4, 37.2, 36.6, 36.3, 34.7, 25.8, 22.9, 16.9, 16.0, 13.5; HRMS (ESI-TOF) m/z Calcd for C₁₅H₂₄NO₃⁺ [M+H]⁺ 266.1756. found 266.1766.

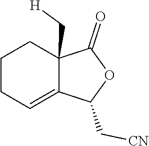 [SMILES: C[C@]12CCCC=C1[C@@H](CC#N)OC2=O]

### 2-((3aS)-3a-methyl-3-oxo-1,3,3a,4,5,6-hexahydroisobenzofuran-1-yl) acetonitrile (6i)

Following General Procedure on 0.1 mmol scale. The product was obtained as colorless oil (60% yield). ¹H NMR (600 MHz, Chloroform-d) δ 5.82 (td, J=3.6, 2.0 Hz, 1H), 5.19 (dddd, J=8.1, 5.2, 2.9, 2.0 Hz, 1H), 2.95 (dd, J=17.1, 5.3 Hz, 1H), 2.82 (dd, J=17.0, 5.0 Hz, 1H), 2.25 (ddtd, J=19.2, 7.0, 3.5, 1.3 Hz, 1H), 2.15 (dddd, J=19.1, 13.5, 6.6, 3.1 Hz, 1H), 1.94 (dt, J=12.9, 3.4 Hz, 1H), 1.85 (ddtd, J=14.1, 6.9, 3.5, 1.3 Hz, 1H), 1.72 (tddd, J=14.1, 10.4, 6.9, 3.5 Hz, 1H), 1.51 (td, J=13.5, 3.7 Hz, 1H), 1.37 (s, 3H); ¹³C NMR (151 MHz, CDCl₃) δ 178.4, 137.8, 122.6, 115.5, 74.6, 41.4, 28.5, 24.1, 22.5, 21.8, 17.0; HRMS (ESI-TOF) m/z Calcd for C₁₁H₁₄NO₂⁺ [M+H]⁺ 192.1025. found 192.1031.

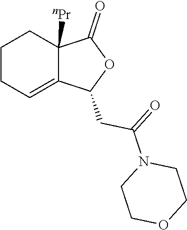

### (7aS)-3-(2-morpholino-2-oxoethyl)-7a-propyl-5,6,7,7a-tetrahydroisobenzofuran-1 (3H)-one (6k)

Following General Procedure on 0.1 mmol scale. The product was obtained as colorless oil (59% yield). ¹H NMR (600 MHz, Chloroform-d) § 5.64 (dt, J=4.0, 2.1 Hz, 1H), 5.53 (dp, J=7.6, 2.4 Hz, 1H), 3.78-3.66 (m, 5H), 3.63-3.42 (m, 3H), 2.77 (dd, J=15.5, 7.4 Hz, 1H), 2.68 (dd, J=15.5, 4.5 Hz, 1H), 2.25-2.16 (m, 1H), 2.05 (ttd, J=11.9, 8.4, 7.5, 3.1 Hz, 2H), 1.78-1.60 (m, 4H), 1.50-1.29 (m, 3H), 0.95-0.86 (m, 3H); ¹³C NMR (151 MHz, CDCl₃) δ 178.9, 168.2, 140.3, 120.7, 78.1, 66.9, 66.7, 46.4, 42.3, 38.2, 37.0, 26.7, 23.9, 17.0, 14.5; HRMS (ESI-TOF) m/z Calcd for C₁₇H₂₆NO₄⁺ [M+H]⁺ 308.1862. found 308.1865.

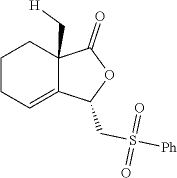 [SMILES: C[C@]12CCCC=C1[C@@H](CS(=O)(=O)c1ccccc1)OC2=O]

### (7aS)-7a-methyl-3-((phenylsulfonyl)methyl)-5,6,7,7a-tetrahydroisobenzofuran-1 (3H)-one (6m)

Following General Procedure on 0.1 mmol scale. The product was obtained as colorless oil (61% yield). ¹H NMR (600 MHz, Chloroform-d) δ 7.96 (ddt, J=9.9, 7.2, 1.3 Hz, 2H), 7.72-7.66 (m, 1H), 7.60 (td, J=8.0, 3.0 Hz, 2H), 5.88 (t, J=3.7 Hz) and 5.73 (td, J=3.6, 2.1 Hz, 1H), 5.53-5.46 (m, 1H) and 5.33 (ddt, J=7.1, 5.3, 1.5 Hz), 3.67-3.57 (m, 1H), 3.48-3.41 (m, 1H), 2.26-2.17 (m, 1H), 2.14-1.99 (m, 1H), 1.86 (tt, J=14.2, 3.4 Hz, 1H), 1.79 (ddtd, J=14.0, 7.1, 3.6, 1.3 Hz, 1H), 1.68 (tddd, J=14.2, 10.4, 6.6, 3.5 Hz, 1H), 1.41-1.25 (m, 4H); ¹³C NMR (151 MHz, CDCl₃) δ 179.0 and 178.5, 139.5 and 139.3, 137.9 and 136.3, 134.39 and 134.36, 129.6 and 129.5, 128.43 and 128.40, 125.9 and 122.0, 75.5 and 74.2, 61.7 and 59.3, 40.8 and 39.6, 29.6 and 28.6, 24.5 and 24.2, 23.9 and 22.2, 17.1 and 17.0; HRMS (ESI-TOF) m/z Calcd for C₁₆H₁₉O₄S⁺ [M+H]⁺ 307.1004. found 307.1005.

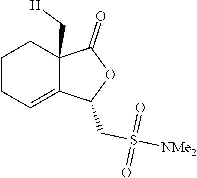 [SMILES: CN(C)S(=O)(=O)C[C@H]1OC(=O)[C@@]2(C)CCCC=C12]

### N,N-dimethyl-1-((3aS)-3a-methyl-3-oxo-1,3,3a,4,5,6-hexahydroisobenzofuran-1-yl) methanesulfonamide (6n)

Following General Procedure on 0.1 mmol scale. The product was obtained as colorless oil (72% yield). ¹H NMR (600 MHz, Chloroform-d) δ 5.92-5.87 (m) and 5.71 (q, J=3.2 Hz, 1H), 5.39 (dp, J=7.8, 2.6 Hz, 1H) and 5.31-5.26 (m), 3.48-3.41 (m, 1H), 3.25-3.18 (m, 1H), 2.94 (d, J=5.3 Hz, 6H), 2.23 (ddt, J=19.3, 6.8, 3.7 Hz, 1H), 2.09 (tddd, J=21.8, 10.3, 7.6, 2.2 Hz, 1H), 1.93 (dt, J=13.1, 3.5 Hz, 1H), 1.82 (ddt, J=14.2, 6.8, 3.6 Hz, 1H), 1.72 (qddd, J=14.2, 10.5, 6.9, 3.5 Hz, 1H), 1.44 (td, J=13.5, 3.6 Hz, 1H), 1.37 (d, J=6.2 Hz, 3H); ¹³C NMR (151 MHz, CDCl₃) δ 179.5 and 178.9, 137.7 and 136.6, 125.6 and 121.8, 76.3 and 74.8, 55.0 and 52.2, 41.0 and 39.8, 37.4, 29.6 and 28.7, 24.8 and 24.2, 23.9 and 22.3, 17.1 and 17.0; HRMS (ESI-TOF) m/z Calcd for C₁₂H₂₀NO₄S⁺ [M+H]⁺ 274.1113. found 274.1119.

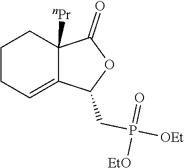

### Diethyl (((3aS)-3-oxo-3a-propyl-1,3,3a,4,5,6-hexahydroisobenzofuran-1-yl)methyl)phosphonate (60)

Following General Procedure on 0.1 mmol scale. The product was obtained as colorless oil (60% yield). ¹H NMR (600 MHz, Chloroform-d) δ 5.74 (p, J=2.2 Hz, 1H), 5.29 (dt, J=12.0, 5.8 Hz, 1H), 4.25-4.05 (m, 4H), 2.31 (ddd, J=19.5, 15.5, 3.9 Hz, 1H), 2.24-2.14 (m, 1H), 2.12-2.01 (m, 3H), 1.80-1.56 (m, 5H), 1.48-1.27 (m, 8H), 0.89 (td, J=7.3, 1.5 Hz, 3H); ¹³C NMR (151 MHz, CDCl₃) δ 178.6, 140.5 and 140.4, 121.4, 75.75 and 75.72, 62.61 and 62.57, 62.03 and 61.99, 45.0, 38.0, 31.0 and 30.1, 26.6, 23.9, 17.8, 16.9, 16.57 and 16.50, 16.54, 14.4; HRMS (ESI-TOF) m/z Calcd for C₁₆H₂₈O₅P⁺ [M+H]⁺ 331.1674. found 331.1679.

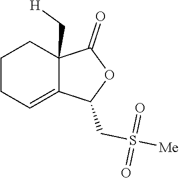 [SMILES: C[C@]12CCCC=C1[C@@H](CS(C)(=O)=O)OC2=O]

### (7aS)-7a-methyl-3-((methylsulfonyl)methyl)-5,6,7,7a-tetrahydroisobenzofuran-1 (3H)-one (61)

Following General Procedure on 0.1 mmol scale. The product was obtained as colorless oil (72% yield). ¹H NMR (600 MHz, Chloroform-d) δ 5.92-5.88 (m) and 5.68 (td, J=3.5, 2.1 Hz, 1H), 5.49 (dtt, J=9.9, 3.1, 1.9 Hz) and 5.39 (dtd, J=10.0, 2.9, 1.6 Hz, 1H), 3.53 (dd, J=15.2, 10.1 Hz) and 3.27 (dd, J=15.3, 9.7 Hz, 1H), 3.39 (ddd, J=15.3, 1.8, 1.3 Hz) and 3.19 (ddq, J=15.2, 2.6, 1.3 Hz, 1H), 3.10 (d, J=1.3 Hz) and 3.06 (d, J=1.3 Hz, 3H), 2.28-2.20 (m, 1H), 2.18-2.14 (m) and 2.12-2.04 (m, 1H), 1.94 (ddt, J=11.9, 8.4, 3.4 Hz, 1H), 1.88-1.81 (m, 1H), 1.73 (ddddt, J=20.9, 14.2, 10.4, 6.7, 3.6 Hz, 1H), 1.50-1.41 (m, 1H), 1.38 (dd, J=3.5, 0.6 Hz, 3H); ¹³C NMR (151 MHz, CDCl₃) δ 179.1 and 178.5, 137.3 and 136.3, 125.9 and 122.0, 76.2 and 74.8, 60.9 and 57.7, 43.1 and 42.8, 40.8 and 39.7, 29.5 and 28.7, 24.8 and 24.2, 23.9 and 22.3, 17.1 and 17.0; HRMS (ESI-TOF) m/z Calcd for C₁₁H₁₇O₄S⁺ [M+H]⁺ 245.0848. found 245.0844.

### 2.6 Synthetic Application

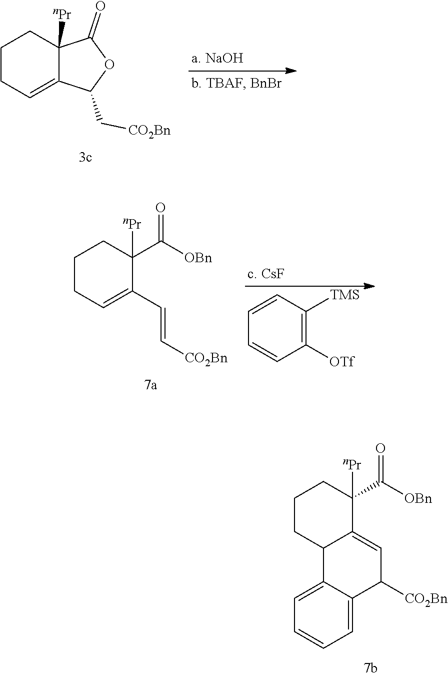

To a solution of 3c (1.0 mmol) in EtOH/H₂O (5.0/5.0 mL) was added NaOH (4.0 eq), then the mixture was heated to reflux until TLC showed that the 3c was fully consumed. The reaction mixture was cooled down to room temperature, and 1M HCl was added until the pH value to about 3, followed by filtration through a pad of celite, and the solvent was removed under vacuum¹. The residue was dissolved in 3 mL tetrabutylammonium fluoride solution (1.0 M in THF) and Benzyl bromide (2.2 mmol) was added. The mixture was stirred under room temperature for 4 hour before 20 mL of H₂O was added. The mixture was extracted with DCM and the organic layers were combined and concentrated under vacuum. 7a was purified by flash chromatography. (83% over 2 steps).

To a screw-cap schlenk tube the 7a (0.2 mmol), caesium fluoride (2.0 mmol), acetonitrile (2 ml) and 2-(trimethylsilyl)phenyl trifluoromethanesulfonate (0.22 mmol) were added. The resulting mixture was stirred at room temperature under argon for 12 h. The reaction mixture was extracted with ethyl acetate (25 mL) and filtered through a short pad of silica gel and eluted with ethyl acetate (25 mL). The solvent was removed under reduced pressure and the crude mixture was purified by flash column chromatography to afford the desired product 7b (87.12 mg, 88%)².

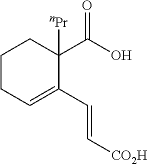

### (S,E)-2-(2-carboxyvinyl)-1-propylcyclohex-2-ene-1-carboxylic acid (7a′)

Following General Procedure on 0.1 mmol scale. The product was obtained as colorless solid. ¹H NMR (600 MHz, Methanol-d₄) δ 7.33 (dd, J=16.0, 1.1 Hz, 1H), 6.42-6.36 (m, 1H), 5.87 (d, J=16.0 Hz, 1H), 2.28-2.13 (m, 2H), 2.07-2.00 (m, 1H), 1.79-1.66 (m, 5H), 1.37-1.27 (m, 1H), 1.21-1.09 (m, 1H), 0.90 (t, J=7.3 Hz, 3H); ¹³C NMR (151 MHz, MeOD) δ 180.6, 171.7, 149.3, 138.8, 138.5, 118.7, 50.3, 40.8, 34.2, 28.2, 20.8, 19.6, 16.0; HRMS (ESI-TOF) m/z Calcd for C₁₃H₁₈O₄⁺ [M+H]⁺ 239.1283. found 239.1284.

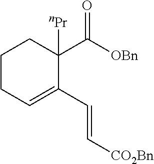

### Benzyl (S,E)-2-(3-(benzyloxy)-3-oxoprop-1-en-1-yl)-1-propylcyclohex-2-ene-1-carboxylate (7a)

Following General Procedure on 0.1 mmol scale. The product was obtained as colorless oil (83% yield). ¹H NMR (600 MHz, Chloroform-d) δ 7.46-7.20 (m, 11H), 6.40-6.33 (m, 1H), 5.88 (d, J=16.0 Hz, 1H), 5.29-5.09 (m, 4H), 2.24 (dq, J=19.4, 5.0 Hz, 1H), 2.19-2.11 (m, 1H), 2.09-2.02 (m, 1H), 1.82 (qdd, J=14.0, 11.2, 5.1 Hz, 2H), 1.72 (ddd, J=13.4, 10.4, 4.0 Hz, 1H), 1.65 (ttd, J=8.6, 5.3, 3.3 Hz, 2H), 1.37-1.21 (m, 1H), 1.16 (tdd, J=13.1, 7.3, 4.0 Hz, 1H), 0.89 (t, J=7.4 Hz, 3H); ¹³C NMR (151 MHz, CDCl₃) δ 175.7, 167.0, 146.7, 137.4, 136.3, 135.9, 135.9, 128.5, 128.5, 128.2, 128.1, 128.1, 128.1, 116.3, 66.6, 66.1, 48.3, 38.4, 32.1, 26.2, 18.7, 17.5, 14.6; HRMS (ESI-TOF) m/z Calcd for C₂₇H₃₁O₄⁺ [M+H]⁺ 419.2217. found 419.2219.

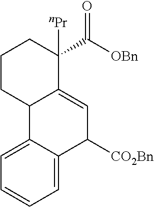

### Dibenzyl (1S,4aR,9R)-1-propyl-1,2,3,4,4a,9-hexahydrophenanthrene-1,9-dicarboxylate (7b)

Following General Procedure on 0.1 mmol scale. The product was obtained as colorless oil (88% yield). ¹H NMR (600 MHz, Chloroform-d) δ 7.39-7.30 (m, 8H), 7.27-7.20 (m, 4H), 7.05 (dd, J=7.0, 2.5 Hz, 2H), 5.33 (d, J=1.4 Hz, 1H), 5.21 (d, J=2.3 Hz, 2H), 5.14 (d, J=12.6 Hz, 1H), 4.98 (d, J=12.6 Hz, 1H), 3.98 (S, 1H), 3.43 (ddd, J=12.6, 4.8, 1.5 Hz, 1H), 2.41 (dd, J=13.0, 3.9 Hz, 1H), 2.14-2.01 (m, 1H), 1.95-1.74 (m, 5H), 1.49-1.30 (m, 2H), 1.05 (dddd, J=19.8, 12.5, 7.2, 4.9 Hz, 1H), 0.85 (t, J=7.3 Hz, 3H); ¹³C NMR (151 MHz, CDCl₃) δ 174.9, 174.8, 143.4, 138.4, 135.9, 135.2, 133.3, 128.5, 128.4, 128.3, 128.2, 128.1, 128.1, 127.5, 127.3, 126.8, 126.0, 120.0, 72.5, 67.6, 66.4, 54.3, 36.5, 36.5, 35.7, 32.1, 21.5, 18.1, 14.6; HRMS (ESI-TOF) m/z Calcd for C₃₃H₃₄O₄Na⁺ [M+Na]⁺ 549.2253. found 549.2250.

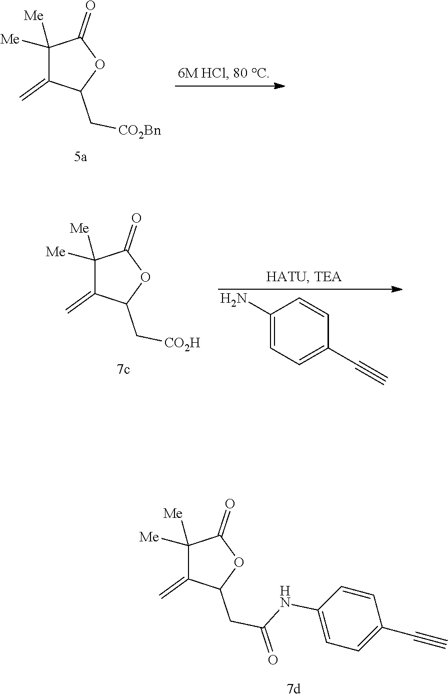

To a solution of 6M HCl (10 mL) was added 5a (0.5 mmol) then the mixture was heated to 80° C. until TLC showed that the 5a was fully consumed. The reaction mixture was cooled down to room temperature, then extracted with DCM. The organic phase was dried with anhydrous Na₂SO₄, and the solvent was removed under vacuum. The residue was purified by column chromatography to afford 7c (86.5 mg, 94%)¹.

To a solution of 7c (0.3 mmol) and 4-ethynylaniline (0.35 mmol) in DCM (10 mL) and TEA (0.9 mmol) was added HATU (0.6 mmol) then the mixture was stirred at room temperature overnight. Saturated ammonium chloride solution was added to the solution and extracted with DCM. The combined organic layers were washed with brine and dried over Na₂SO₄ and concentrated in vacuo. The residue was purified by column chromatography to afford 7d (69.8 mg, 82%).

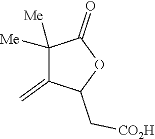

### (R)-2-(4,4-dimethyl-3-methylene-5-oxotetrahydrofuran-2-yl) acetic acid (7c)

Following General Procedure on 0.1 mmol scale. The product was obtained as colorless oil (94% yield). ¹H NMR (600 MHz, Chloroform-d) δ 5.40 (ddt, J=7.0, 4.6, 2.4 Hz, 1H), 5.12 (ddd, J=6.4, 2.4, 1.4 Hz, 2H), 2.92 (dd, J=17.0, 4.5 Hz, 1H), 2.80 (dd, J=17.0, 7.3 Hz, 1H), 1.34 (d, J=3.2 Hz, 6H); ¹³C NMR (151 MHz, CDCl₃) δ 180.4, 175.3, 152.4, 107.5, 76.3, 43.1, 39.7, 27.0, 24.8; HRMS (ESI-TOF) m/z Calcd for C₉H₁₂O₄⁺ [M+H]⁺ 185.0814. found 185.0812.

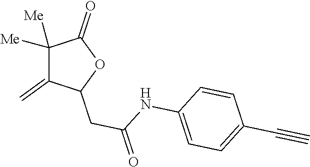 [SMILES: C#Cc1ccc(NC(=O)CC2OC(=O)C(C)(C)C2=C)cc1]

### (R)-2-(4,4-dimethyl-3-methylene-5-oxotetrahydrofuran-2-yl)-N-(4-ethynylphenyl) acetamide (7d)

Following General Procedure on 0.1 mmol scale. The product was obtained as white solid (82% yield). ¹H NMR (600 MHz, Chloroform-d) δ 7.93 (s, 1H), 7.52-7.45 (m, 2H), 7.44-7.38 (m, 2H), 5.50 (ddt, J=8.4, 4.0, 2.4 Hz, 1H), 5.20-5.09 (m, 2H), 3.04 (s, 1H), 2.87 (dd, J=15.2, 3.8 Hz, 1H), 2.74 (dd, J=15.2, 8.3 Hz, 1H), 1.34 (d, J=13.1 Hz, 6H); ¹³C NMR (151 MHz, CDCl₃) δ 180.7, 167.1, 152.0, 138.1, 133.0, 119.7, 118.1, 108.0, 83.4, 77.7, 77.0, 43.5, 43.1, 26.8, 24.9; HRMS (ESI-TOF) m/z Calcd for C₁₇H₁₈NO₃⁺ [M+H]⁺ 284.1287. found 284.1292.

### REFERENCES

1. Zhuang, Z.; Yu, C.-B.; Chen, G.; Wu, Q.-F.; Hsiao, Y.; Joe, C. L.; Qiao, J. X.; Poss, M. A.; Yu, J.-Q. Ligand-enabled β-C(sp³)-H olefination of free carboxylic acids. J. Am. Chem. Soc. 2018, 140, 10363-10367.
2. Wang, Y.-C.; Huang, Y.-H.; Tsai, H.-C.; Basha, R. S.; Chou, C.-M. Palladium-catalyzed proaromatic C (alkenyl)-H olefination: synthesis of densely functionalized 1,3-dienes. Org. Lett. 2020, 22, 6765-6770.

The foregoing disclosure has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

This application refers to various issued patents, published patent applications, journal articles, and other publications, each of which are incorporated herein by reference.

What is claimed is:

1. A method of forming a β-alkylidene-γ-lactone, comprising treating an a-substituted carboxylic acid with a vinyl reagent containing an electron withdrawing group (EWG) in the presence of a Pd source/Ligand (L) catalyst.

2. The method of claim 1, wherein the method of forming a β-alkylidene-γ-lactone occurs according to the following reaction scheme: 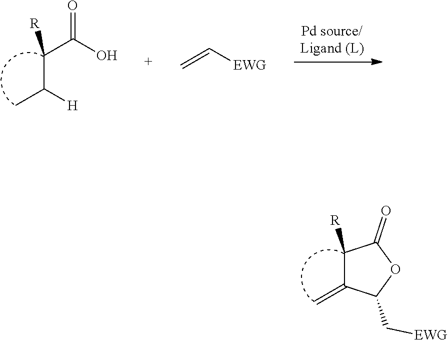
wherein 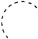
represents a substituted or unsubstituted C₅-C₁₂ ring system;
R is optionally substituted —(C₁-C₆)alkyl, aryl, —(C₁-C₆) haloalkyl, —O(C₁-C₆)alkyl, cyclocalkyl, —(C₁-C₆)alkylcycloalkyl, or —(C₁-C₆)alkylaryl;
EWG is —CO₂Bn, —CN, —CO₂R′, —C(═O)R′, —SO₂R′, or —P(O)(OR′)₂; and
each R′ is independently optionally substituted —(C₁-C₁₂)alkyl, —O(C₁-C₁₂)alkyl,
cycloalkyl, heterocycloalkyl, —N((C₁-C₆)alkyl)₂, or Ph.

3. The method of either claim 1 or claim 2, wherein the method of forming a β-alkylidene-γ-lactone occurs according to the following reaction scheme: 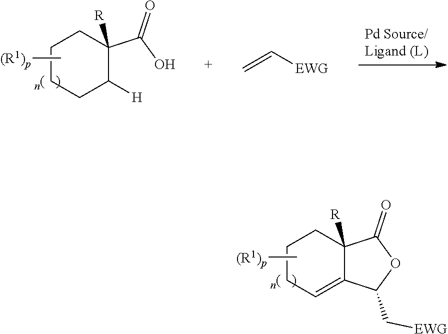
wherein:
R is optionally substituted —(C₁-C₆)alkyl, aryl, —(C₁-C₆) haloalkyl, —O(C₁-C₆)alkyl, cyclocalkyl, —(C₁-C₆)alkylcycloalkyl, or —(C₁-C₆)alkylaryl;
EWG is —CO₂Bn, —CN, —CO₂R′, —C(═O)R′, —SO₂R′, or —P(O)(OR′)₂;
each R′ is independently optionally substituted —(C₁-C₁₂)alkyl, —O(C₁-C₁₂)alkyl,
cycloalkyl, heterocycloalkyl, —N((C₁-C₆)alkyl)₂, or Ph;
each R¹ is independently optionally substituted —(C₁-C₆)alkyl or —O(C₁-C₆)alkyl;
n is 0-7; and
p is 0-4.

4. The method of claim 3, wherein n is 1; and p is 0 or 1.

5. The method of claim 3, wherein n is 1 or 2; and p is 0, 1, or 2.

6. The method of claim 3, wherein n is 1, and p is 0, 1, or 2.

7. The method of claim 3, wherein n is 1; and p is 0.

8. The method of claim 3, wherein n is 1; and p is 1.

9. The method of claim 3, wherein n is 1; and p is 2.

10. The method of claim 3, wherein n is 1; and p is 0.

11. The method of claim 3, wherein n is 1, 2, or 3; and p is 0.

12. The method of any one of claims 1-11, wherein the Ligand (L) is selected from the group consisting of:

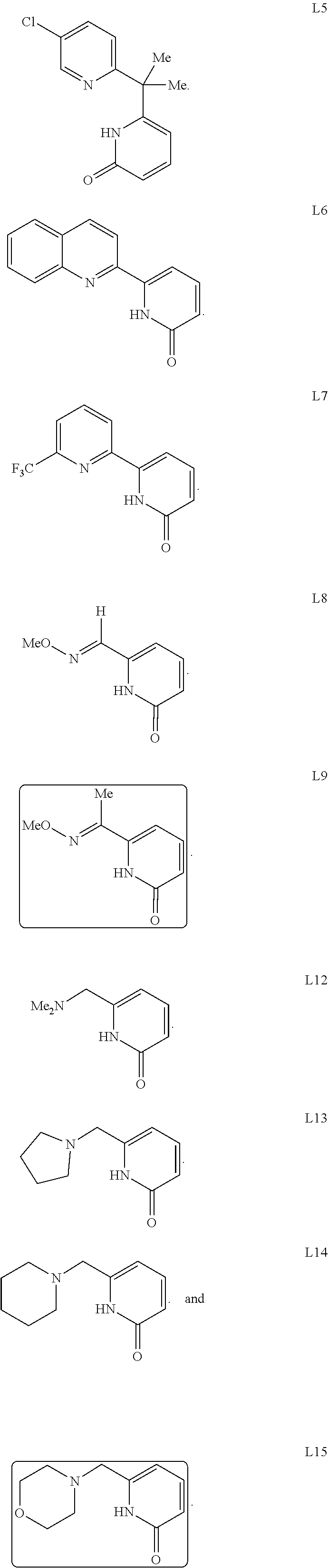

13. The method of any one of claims 1-12, wherein the Ligand (L) is L6 or L9, or L15.

14. The method of any one of claims 1-13, wherein the Ligand (L) is L9 or L15.

15. The method of any one of claims 1-13, wherein the Ligand (L) is L6.

16. The method of any one of claims 1-14, wherein the Ligand (L) is L9.

17. The method of any one of claims 1-14, wherein the Ligand (L) is L15.

18. The method of any one of claims 1-17, wherein the Ligand (L) is present in approximately 10-20 mol %.

19. The method of any one of claims 1-18, wherein the Ligand (L) is present in approximately 13 mol %.

20. The method of any one of claims 1-19, wherein the Pd source is a Pd salt.

21. The method of any one of claims 1-20, wherein the Pd source is Pd(OAc)₂, PdCl₂C(CH₃CN)₂, Pd(TFA)₂, Pd(dba)₂, Pd(PhCN)₂Cl₂, or Pd(CH₃CN)₄(OTf)₂.

22. The method of any one of claims 1-21, wherein the Pd source is Pd(OAc)₂.

23. The method of any one of claims 1-21, wherein the Pd source is PdCl₂C(CH₃CN)₂.

24. The method of any one of claims 1-21, wherein the Pd source is Pd(TFA)₂.

25. The method of any one of claims 1-21, wherein the Pd source is Pd(dba)₂.

26. The method of any one of claims 1-21, wherein the Pd source is Pd(PhCN)₂Cl₂.

27. The method of any one of claims 1-21, wherein the Pd source is Pd(CH₃CN)₄(OTf)₂.

28. The method of any one of claims 1-27, wherein the Pd source is present in approximately 5-20 mol %.

29. The method of any one of claims 1-28, wherein the Pd source is present in approximately 10 mol %.

30. The method of any one of claims 1-29, wherein the method of forming a β-alkylidene-γ-lactone occurs in the presence of an oxidant.

31. The method of claim 30, wherein the oxidant is an Ag salt.

32. The method of claim 31, wherein the Ag salt is Ag₂CO₃, AgOAc, AgF, AgTFA, Ag₂₀, or Ag₃PO₄.

33. The method of claim 32, wherein the Ag salt is Ag₂CO₃.

34. The method of claim 32, wherein the Ag salt is AgOAc.

35. The method of claim 32, wherein the Ag salt is AgF.

36. The method of claim 32, wherein the Ag salt is AgTFA.

37. The method of claim 32, wherein the Ag salt is Ag₂O.

38. The method of any one of claims 30-37, wherein the oxidant is present in approximately 1.0-3.0 equivalents.

39. The method of any one of claims 30-38, wherein the oxidant is present in approximately 2.0 equivalents.

40. The method of any one of claims 1-39, wherein the method of forming a B-alkylidene-γ-lactone occurs in the presence of an HFIP solvent mixture.

41. The method of claim 40, wherein the HFIP solvent mixture is approximately 10:1 HFIP:MeCN.

42. The method of claim 40, wherein the HFIP solvent mixture is approximately 10:1.5 HFIP:MeCN.

43. The method of claim 40, wherein the HFIP solvent mixture is approximately 10:1 HFIP:CH₃ (CH₂)₂CN.

44. The method of claim 40, wherein the HFIP solvent mixture is approximately 20:1 HFIP:MeCN.

45. The method of claim 40, wherein the HFIP solvent mixture is approximately 10:1 HFIP:DMSO.

46. The method of any one of claims 1-45, wherein the method of forming a β-alkylidene-γ-lactone occurs according to the following reaction scheme: 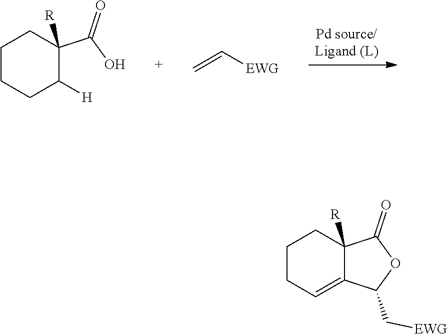
wherein R is optionally substituted —(C₁-C₆)alkyl, aryl, —(C₁-C₆) haloalkyl, —O(C₁-C₆)alkyl, cyclocalkyl, —(C₁-C₆)alkylcycloalkyl, or —(C₁-C₆)alkylaryl.

47. The method of claim 46, wherein R is Me.

48. The method of claim 46, wherein R is iPr.

49. The method of any one of claim 1-2, 15, 18-22, 28-33, or 38-41, wherein the method of forming a β-alkylidene-γ-lactone occurs according to the following reaction scheme:

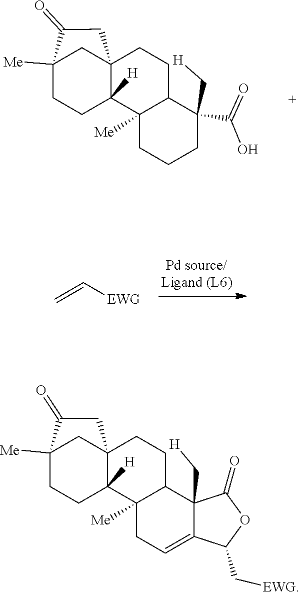

50. The method of any one of claims 1-2, 15, 18-22, 28-33, or 38-41, wherein the method of forming a β-alkylidene-γ-lactone occurs according to the following reaction scheme:

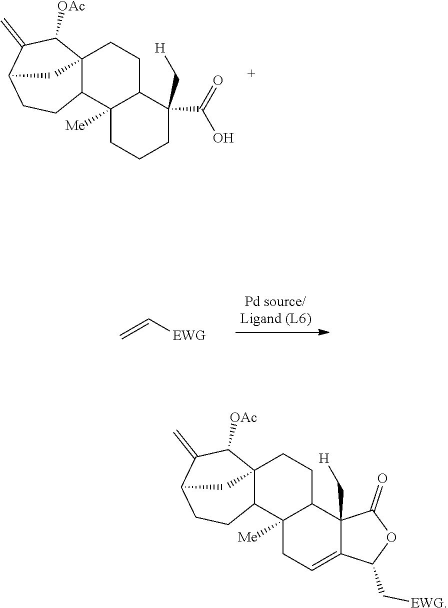

51. The method of any one of claims 1-50, wherein EWG is —CO₂Bn.

52. The method of any one of claim 1-13, 18-22, 28-33, or 38-41, wherein the β-alkylidene-γ-lactone is selected from the group consisting of Formulae (3a-3aa):

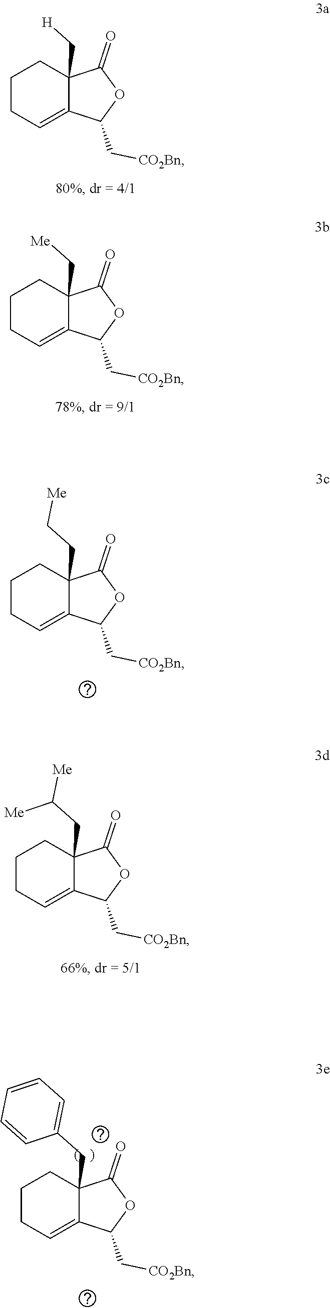

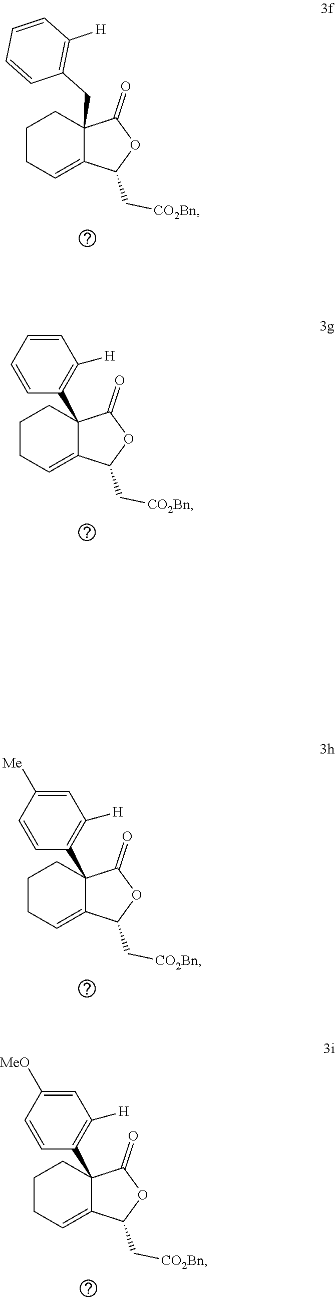 [SMILES: O=C(C[C@H]1OC(=O)[C@@]2(Cc3ccccc3)CCCC=C12)OCc1ccccc1 ; O=C(C[C@H]1OC(=O)[C@@]2(c3ccccc3)CCCC=C12)OCc1ccccc1 ; Cc1ccc([C@]23CCCC=C2[C@@H](CC(=O)OCc2ccccc2)OC3=O)cc1 ; COc1ccc([C@]23CCCC=C2[C@@H](CC(=O)OCc2ccccc2)OC3=O)cc1]

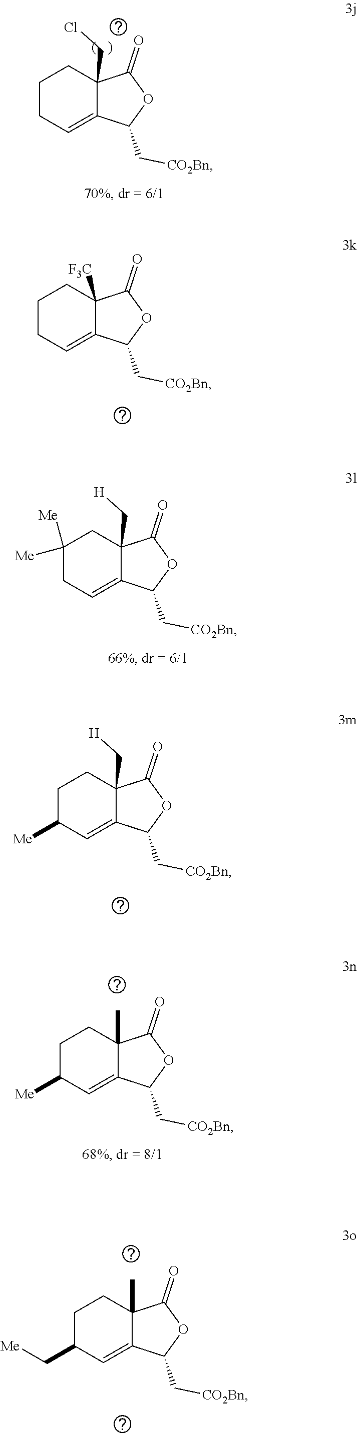

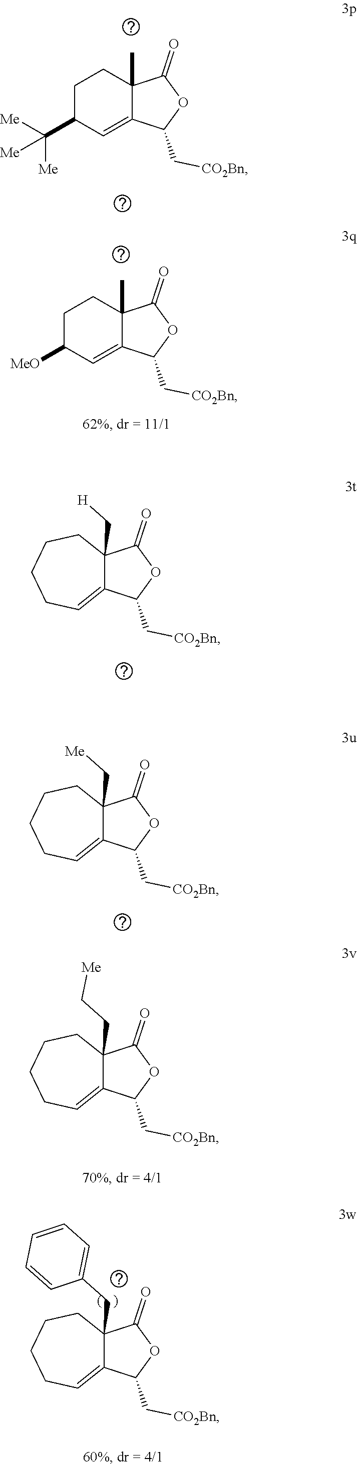

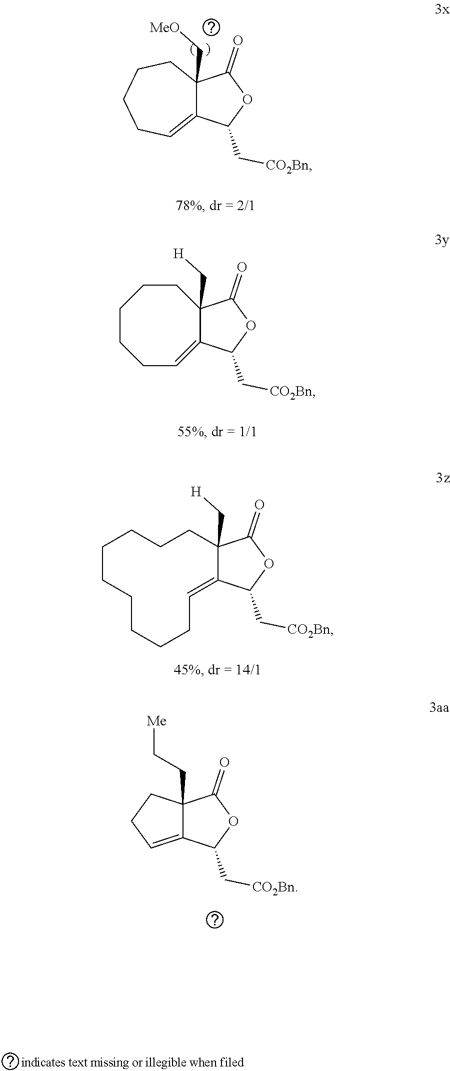

53. The method of claim 1, wherein the method of forming a β-alkylidene-γ-lactone occurs according to the following reaction: 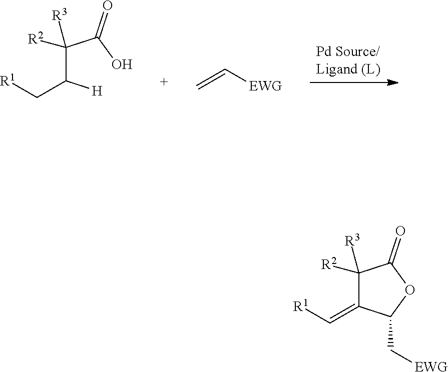
wherein:
R¹ is H, (C₁-C₆)alkyl, or —(C₁-C₆)alkylO(C₁-C₆)alkyl;
R² and R³ are independently —(C₁-C₆)alkyl, —(C₁-C₆)alkylPh, —(C₁-C₆)alkylcycloalkyl, —(C₁-C₆)alkylO(C₁-C₆)alkyl, or halo(C₁-C₆)alkyl;
EWG is —CO₂Bn, —CN, —CO₂R′, —C(═O)R′, —SO₂R′, or —P(O)(OR′)₂; and
each R′ is independently optionally substituted —(C₁-C₁₂)alkyl, —O(C₁-C₁₂)alkyl,
cycloalkyl, heterocycloalkyl, —N((C₁-C₆)alkyl)₂, or Ph.

54. The method of claim 53, wherein the method of forming a β-alkylidene-γ-lactone occurs in the presence of NaOAc.

55. The method of claim 54, wherein the NaOAc is present in approximately 2.0 equivalents.

56. The method of any one of claims 53-55, wherein EWG is CO₂Bn.

57. The method of claim 53, wherein the α-substituted aliphatic carboxylic acid (1.0 eq.) is treated with benzyl acrylate (2.0 eq.) in the presence of Pd(OAc)₂ (10 mol %), L9 (13 mol %), NaOAc (2.0 eq.), and Ag₂CO₃ (2.0 eq.), in HFIP/MeCN (19/1).

58. The method of claim 1, wherein the method of forming a β-alkylidene-γ-lactone occurs according to the following reaction scheme: 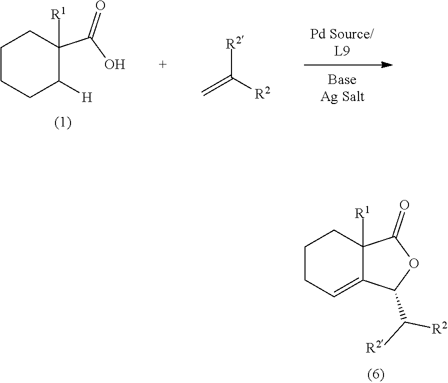
wherein:
R¹ is optionally substituted —(C₁-C₆)alkyl, aryl, —(C₁-C₆) haloalkyl, —O(C₁-C₆)alkyl, cyclocalkyl, —(C₁-C₆)alkylcycloalkyl, or —(C₁-C₆)alkylaryl; R² is H or —CH₂CO₂ (C₁-C₆)alkyl;
R^{2′} is —CO₂Rₐ, —CN, —C(═O)R_{b}, —S(═O)₂ (C₁-C₆)alkyl, —S(═O)₂Ph, —S(═O)₂N(C₁-C₆alkyl)₂, or —P(═O)(O(C₁-C₆)alkyl)₂;
Rₐ is —(C₁-C₁₂)alkyl, cycloalkyl, or —(C₁-C₆)alkyl-O—(C₁-C₆)alkyl; and
R_{b} is —N(C₁-C₆ alkyl)₂ or heterocycloalkyl.

59. The method of claim 58, wherein the Pd source is Pd(OAc)₂.

60. The method of either claim 58 or claim 59, wherein the Ag salt is Ag₂CO₃.

61. The method of any one of claims 58-60, wherein the Base is KF.

62. The method of any one of claims 58-60, wherein the Base is LiF.

63. The method of any one of claims 58-62, wherein the solvent mixture is approximately 10:1.0 HFIP:MeCN.

64. The method of any one of claims 58-62, wherein the solvent mixture is approximately 10:1.5 HFIP:MeCN.

65. The method of any one of claims 58-64, wherein R² is H.

66. The method of claim 58, wherein the α-substituted aliphatic carboxylic acid substrate (1.0 eq.) is treated with CH₂C(R²)(R^{2′})(2.0 eq.) in the presence of Pd(OAc)₂ (10 mol %), L9 (13 mol %), Base (2.0 eq.), and Ag₂CO₃ (2.0 eq.), in HFIP/MeCN.

67. A method of the synthesis of the compound of Formula (7b) from the γ-lactone product of Formula (3c) prepared according to the method of claim 48, comprising the steps of:

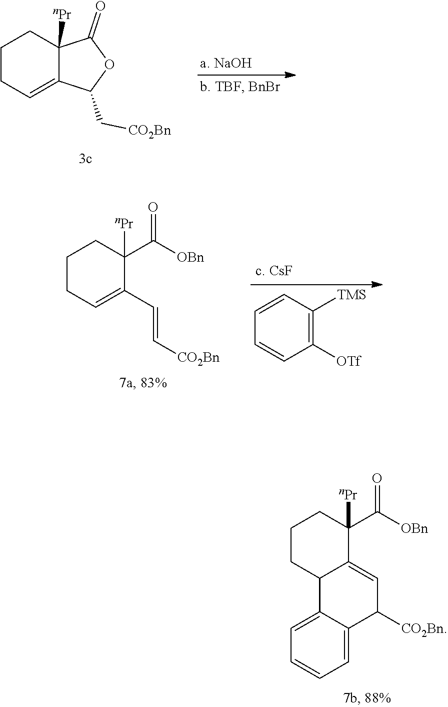

68. A method of the synthesis of Formula (7d) from the γ-lactone product of Formula (5a) prepared according to the method of claim 53, comprising the steps of:

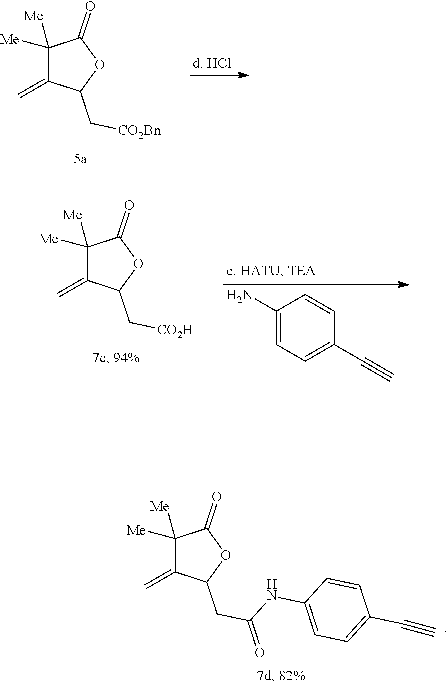

69. Any method of synthesis of β-alkylidene-γ-lactones via dehydrogenation-olefination-lactonization of free aliphatic carboxylic acid substrates, including natural products isosteviol and grandiflorolic acid, or method of further synthesis involving the product β-alkylidene-γ-lactones as disclosed herein.
